(19) United States
(12) Patent Application Publication
(10) Pub. No.: US 2026/0049154 A1
(43) Pub. Date: Feb. 19, 2026

(54) IMMUNO-NANOPARTICLES, AND RELATED CELLS, COMPOSITIONS, METHODS AND SYSTEMS

(71) Applicant: LAWRENCE LIVERMORE NATIONAL SECURITY, LLC, Livermore, CA (US)

(72) Inventors: Claire Jane ROBERTSON, Oakland, CA (US); Wei HE, Pleasanton, CA (US); Matthew A COLEMAN, Oakland, CA (US)

(21) Appl. No.: 19/296,737

(22) Filed: Aug. 11, 2025

Related U.S. Application Data
(60) Provisional application No. 63/682,737, filed on Aug. 13, 2024.

(51) Int. Cl. C07K 16/30; A61K 9/51; A61K 38/00; C07K 14/705; C07K 14/725; C07K 16/28

(52) U.S. Cl. CPC C07K 16/30; A61K 9/5123; A61K 9/5169; A61K 38/00; C07K 14/7051; C07K 14/70521; C07K 16/2851; C07K 16/2887; C07K 2317/53; C07K 2317/622; C07K 2319/03

(57) ABSTRACT

An immuno-nanolipoprotein particle (Immuno-NLP) comprising one or more membrane forming lipids, a scaffold protein and an immune cell transmembrane protein, and methods of using immuno-nanolipoprotein particles to deliver immune cell transmembrane protein to immune cells, and related compositions, methods, and systems.

### CROSS REFERENCE TO RELATED APPLICATIONS

The present application claims priority to U.S. provisional application No. 63/682,737, entitled “Immuno-Nanoparticles and Related Cells, Compositions, Methods and Systems,” filed on Aug. 13, 2024 with docket number IL-13908, the content of which is incorporated herein by reference in its entirety.

### STATEMENT OF GOVERNMENT GRANT

The invention was made with Government support under Contract No. DE-AC52-07NA27344 between the U.S. Department of Energy and Lawrence Livermore National Security, LLC, for the operation of Lawrence Livermore National Security. The invention was also made with Government support under grant 22-DR-001 issued awarded by Lawrence Livermore National Laboratory, Laboratory Directed Research Program. The Government may have certain rights to the invention.

### REFERENCE TO SEQUENCE LISTING

Further, the computer readable form of the sequence listing of the ASCII (XML) text file IL-13908-02-Seq-ID.xml, created on Aug. 11, 2025, with a size of 10,486 bytes, is incorporated herein by reference in its entirety.

### FIELD

The present disclosure relates to the field of immunology and to the use of immunoreceptors or other transmembrane protein of immune cells, and in particular chimeric antigen receptors (CARs) in combination with immune cells for cell targeting. In particular, the present disclosure relates to nanolipoprotein particles (NLPs) comprising one or more transmembrane proteins, in particular an immunoreceptor (Immuno-Nanoparticles or Immuno-NLPs) and to the related use to deliver the transmembrane protein or immunoreceptors to immune cells as well as related cells, compositions, methods, and systems.

### BACKGROUND

Transmembrane proteins of immune cells such as immunoreceptors are expressed in cells of the immune system and are involved in the immune response typically by recognizing specific antigens and/or mediating immune responses. Immunoreceptors comprise engineered proteins, such as chimeric antigen receptors (CARs) as well as additional membrane proteins involved in targeting of a cell, such as an immune cell to recognize, or pathogen.

Although progress has been made in connection with the use of immunoreceptors or other transmembrane proteins, in particular when associated with treatments of an individual, challenges still remain for developing methods that result in efficient use of immunoreceptors in connection with cell targeting in particular when associated to treatment of a disease.

### SUMMARY

Provided herein are nanolipoprotein particles (NLPs) obtained by a method designed to increase solubilization of immune cells transmembrane protein and the related loading on NLPs. NLPs obtained by methods of the disclosure to have the minimum cargo loading have been surprisingly found to be required for effective protein transfer and presentation of a functional immune cell transmembrane protein into immune cells (Immuno-NLPs)

Immuno-NLPs of the present disclosure can thus comprise immune cells transmembrane protein such as an immunoreceptor protein and in particular chimeric antigen receptors (CARs) (CAR-NLPs).

Immuno-NLPs of the present disclosure and related cells, compositions, methods, and systems in several embodiments, allow production of NLPs with increased solubility in aqueous solutions and deprived of sources of toxicity for the immune cells, which allow delivery and presentation of functional transmembrane protein and in particular immunoreceptors into immune cells in culture through a non-transgenic cell modification.

According to a first aspect, an immuno-nanolipoprotein particle (Immuno-NLP) is described, comprising: one or more membrane forming lipid, a scaffold protein, and an immune cell transmembrane protein, such as immunoreceptor protein, e.g. a CAR. In the Immuno-NLP, the immune cell transmembrane protein comprises an extracellular domain, a hydrophobic transmembrane domain, and an intracellular domain. In the Immuno-NLP, the one or more membrane forming lipids are arranged in a discoidal membrane lipid bilayer stabilized by the scaffold protein, in a configuration in which the transmembrane protein is attached to the membrane lipid bilayer through interactions of the hydrophobic transmembrane domain of the immunoreceptor with the membrane lipid bilayer, and presents a functional extracellular domain of the immune cell transmembrane protein.

In Immuno-NLP where the transmembrane protein is an immunoreceptor protein, the extracellular domain comprises an extracellular ligand recognition domain (LRD) configured to specifically bind a corresponding ligand and, in the Immuno-NLP, the LRD is in a configuration wherein the LRD specifically binds the corresponding target.

In the Immuno-NLP of the disclosure, the immune cell transmembrane protein is comprised in the NLP which can comprise additional components such as a telodendrimer and additional components identifiable by a skilled person upon reading of the present disclosure.

According to a second aspect, an Immuno-NLP composition is described, comprising one or more immune cell transmembrane proteins, such as immunoreceptor proteins, and one or more NLPs in which one or more membrane forming lipids are arranged in a discoidal membrane lipid bilayer stabilized by the scaffold protein. In the Immuno-NLP composition, an immune cell transmembrane protein of one or more immune cell transmembrane proteins is attached to the membrane lipid bilayer of the NLP of the one or more NLPs through interactions of the hydrophobic transmembrane domain of the transmembrane protein with the membrane lipid bilayer to form an Immuno-NLP of the disclosure. In the Immuno-NLP composition, the immune cell transmembrane protein and the NLP are in a suspension that is ≥50% and possibly ≥90% soluble in a buffered aqueous solutions, wherein in the immune cell transmembrane protein and the NLP are in a molar ratio of immunoreceptor to NLP of at least 1:10. In preferred embodiments the immune cell transmembrane protein is an immunoreceptor protein, more preferably a CAR protein and most preferably a CAR protein comprising an scFv domain.

In some embodiments, the Immuno-NLP composition of the disclosure is provided in an Immuno-NLP treatment formulation comprising type and concentration of components that are non-toxic to immune cells, and thus maximize immune cell viability and functionality. Immuno-NLP treatment formulation can be provided by removing from the Immuno-NLP composition, chemical components known or identified to be capable of interfering with viability and functionality of immune cells, such as imidazole buffers or other protein purification components or insoluble protein aggregates. In some embodiments, the Immuno-NLP treatment formulation is a sterile formulation free from viable microorganisms, such as bacteria, viruses, fungi, and any other potentially infectious agents as will be understood by a skilled person. In preferred embodiments, the Immuno-NLP treatment formulation does not comprise detectable amounts of an insoluble immunoreceptor protein and can be obtained by removing insoluble receptor protein from the Immuno-NLP composition as will be understood by a skilled person.

According to a third aspect, an Immuno-NLP one-pot method and system are described for producing an Immuno-nanolipoprotein particle (Immuno-NLP) and an Immuno-NLP composition of the present disclosure.

The Immuno-NLP one-pot method comprises mixing one or more membrane forming lipids, a polynucleotide coding for the immune cell transmembrane protein (ICTM)(ICTM encoding polynucleotide), and a polynucleotide coding for a scaffold protein (SCP)(SCP encoding polynucleotide) in a cell-free reaction mixture to obtain an Immuno-NLP cell free reaction mixture. In the one-pot method, the Immuno-NLP cell free reaction mixture obtained from the mixing comprises:
i) the ICTM encoding polynucleotide and the SCP encoding polynucleotide in a total polynucleotide concentration from 0.1 μg/mL to 100 μg/mL
ii) the ICTM encoding polynucleotide (ICTM polynucleotide) and the SCP encoding polynucleotide (SCP polynucleotide) at a ICTM polynucleotide:SCP polynucleotide molar ratio of from 1:1 and 120:1
iii) the one or more membrane forming lipids at a total lipid concentration of from 0.1 mg/ml to 100 mg/ml with 10⁻³⁰ mg/ml preferred and
iv) a ratio (weight/weight) of total polynucleotide to lipid within the cell free reaction mix from 0.000001 and 1.

In embodiments, where the Immuno-NLP further comprises a telodendrimer, the reaction mixture further comprises:
v) one or more telodendrimers
vi) at a telodendrimer reaction mixture ratios from 0.01 mg/ml to 1 g/ml with preferred ratios of 10 mg/ml to 40 mg/ml
vii) at a telodendrimer to lipid (weight:weight) ratio within the cell free reaction mix from 0.1 to 10, with 0.5-2 preferred,
viii) at a ratio (weight/weight) of telodendrimer to polynucleotide within the cell free mix from 10⁻⁷ to 0.1.

In embodiments where the ICTM is a CAR, the ICTM polynucleotide is a CAR polynucleotide comprised in the reaction mixture at a CAR polynucleotide:SCP polynucleotide molar ratio. In those embodiments, in the reaction mixture
the total polynucleotide concentration is from 0.1 μg/mL to 100 μg/mL, with a preferred total polynucleotide concentration from 10 μg/mL to 30 μg/mL;
the CAR polynucleotide: SCP polynucleotide molar ratio is from 10:1 to 120:1, with preferred ratios of from 30:1 to 50:1 and
the total lipid concentration is from 0.1 mg/ml to 100 mg/ml with between 10 and 30 mg/ml preferred.

In embodiments, where the ICTM is a CAR, and the Immuno-NLP comprises a telodendrimer, the telodendrimer to cell free reaction mix is from 0.01 mg/ml to 1 g/ml with preferred ratios of 10 mg/ml to 40 mg/ml.

The Immuno-NLP one-pot method further comprises incubating the Immuno-NLP-cell free reaction mixture at a temperature from 15° C. to 30° C., and for a time from one (1) hour to 72 hours, the time and the temperature selected to allow protein synthesis of the scaffold protein and the transmembrane protein, self-assembly of the scaffold protein and the one or more membrane forming lipids into a discoidal membrane lipid bilayer stabilized by the scaffold protein, and self-assembly of the nanolipoprotein particle comprising the transmembrane protein within said discoidal membrane lipid bilayer, the membrane lipid bilayer attaching the transmembrane protein through interaction of a hydrophobic region of the CAR with the membrane lipid bilayer.

In some embodiments, the Immuno-NLP one-pot method of the disclosure can be performed for a time and under condition(s) to obtain an Immuno-NLP and an Immuno-NLP composition comprising immunoreceptor and NLPs in molar ratio of immunoreceptor to NLP of at least 1:10, up to 60:10.

In preferred embodiments of the Immuno-NLP one-pot method, the incubating can be performed at a temperature from 18° C. to 22° C. and most preferably at 19.5° C. to 20.5° C., to provide an Immuno-NLP of the disclosure which is >50% of the ICTM -NLP solution that is soluble in buffered aqueous solutions.

The Immuno-NLP one-pot system of the third aspect comprises one or more membrane forming lipids, an ICTM encoding polynucleotide, coding for an immune cell transmembrane protein, an SCP encoding polynucleotide coding for a scaffold protein and a cell-free reaction mixture for simultaneous combined or sequential use in the mixing of the Immuno-NLP one-pot method of the disclosure to provide an Immuno-NLP and an Immuno-NLP composition according to the second aspect of the present disclosure.

According to a fourth aspect, an Immuno-NLP two-pot method and system are described for producing an immuno-NLP and an Immuno-NLP composition of the disclosure. The method comprises mixing in a first pot cell free reaction mixture, one or more membrane forming lipids with an SCP encoding polynucleotide coding for a scaffold protein for a time and under conditions to allow translation of the scaffold protein in the presence of the one or more membrane forming lipids and self-assembly of a nanolipoprotein particle in a discoidal membrane lipid bilayer formed by the one or more membrane forming lipids and stabilized by the scaffold protein, thus providing an assembled NLP.

In the two-pot method, the first pot cell free reaction mixture comprises:
i) the SCP encoding polynucleotide in a total polynucleotide concentration from 0.1 μg/mL to 100 μg/mL, and
ii) the one or more membrane forming lipids at a total lipid concentration of from 0.1 mg/ml to 100 mg/ml.

In embodiments where the Immuno-NLP comprises one or more telodendrimers, the NLP cell free reaction mixture obtained from the mixing of the two-pot method further comprises a telodendrimer at a telodendrimer to cell free reaction mix ratio of from 0.01 mg/ml tolg/ml with preferred ratios of from 10 mg/ml to 40 mg/ml.

The Immuno-NLP two-pot method further comprises incubating in a second pot cell free reaction mixture the NLP assembled in the first pot reaction mixture with an ICTM encoding polynucleotide for a time and under conditions to allow translation of the ICTM for a time and under conditions allowing that expression of the ICTM protein and attachment of the ICTM protein within the discoidal membrane lipid bilayer of the assembled NLP assembled in the first pot reaction mixture, the membrane lipid bilayer attaching the immune cell transmembrane protein through interaction of a hydrophobic region of the immune cell transmembrane protein with the membrane lipid bilayer.

In the Immuno-NLP two-pot method, the second pot cell free reaction mixture comprises:
i) the NLPs formed in the first reaction at a concentration from 0.01 mg/ml to 10 mg/ml
ii) an ICTM encoding polynucleotide encoding for the immune cell transmembrane protein in a polynucleotide concentration from 0.1 μg/mL to 100 μg/mL.

In some embodiments, the immuno-NLP two-pot method of the disclosure can be performed for a time and under condition(s) to obtain an Immuno-NLP composition of the disclosure, comprising immune cell transmembrane protein and NLPs in molar ratio immune cell transmembrane protein to NLP of at least 1:10 and up to 10:10 or higher as will be understood by a skilled person upon reading of the disclosure

The Immuno-NLP two pot system to provide an Immuno-NLP according to the present disclosure, comprises one or more membrane forming lipids, an SCP polynucleotide coding for a scaffold protein and a cell free reaction mixture for simultaneous combined or sequential use to provide a pre-assembled NLP in the two-pot method of the disclosure. The Immuno-NLP two-pot system of the disclosure further comprises an ITCM polynucleotide encoding for an immune cell transmembrane protein and a cell free reaction mixture for simultaneous combined or sequential use to provide an Immuno-NLP of the disclosure comprising the immune cell transmembrane protein attached to the membrane lipid discoidal bilayer of the assembled-NLP from the incubating.

According to a fifth aspect, an Immuno-NLP transfer method and systems are described for transferring an immune cell transmembrane protein into an immune cell. The Immuno-NLP transfer method comprises contacting the immune cell with an immuno-NLP of the disclosure, in the absence of detectable concentrations of an agent toxic for the immune cells and under sterile conditions, the contacting performed for a time and under conditions resulting in the transfer of the immune cell transmembrane protein on the membrane of the immune cell. The Immuno-NLP transfer method further comprises treating the immune cells with reagents to induce cell activation, the treating performed before and/or concurrently with the contacting treating the immune cells with pre- or concurrent stimulation of the immune cells with reagents to induce cell activation. In preferred embodiments, the immune cell transmembrane protein is an immunoreceptor, more preferably a CAR protein.

In some embodiments of an Immuno-NLP transfer method, the contacting can be performed by culturing activated immune cells with Immuno-NLP and/or a sterile Immuno-NLP treatment formulation comprising at least 10 nanomolar and possibly at least 17.5 nanomolar concentration of immunoreceptor protein for a sufficient time to obtain detectable presence of immunoreceptor protein on the membrane of the immune cells, in preferred embodiments the culturing is performed from one (1) hour to 10 days, and more preferable from 6 hours to 48 hours, as will be understood by a skilled person upon reading of the present disclosure.

The Immuno-NLP transfer system of the disclosure comprises an Immuno-NLP of the disclosure and/or an Immuno-NLP transfer formulation of the disclosure in combination with immune cells, and agents for the activation of the immune cells, the agents in amounts configured to enable activation of the immune cells upon contacting of the agents with the immune cells, the immuno-NLP and/or Immuno-NLP transfer formulation in amounts configured to enable the transfer of the immune cell transmembrane protein onto the membrane of the immune cells following contacting with the immune cell according to an Immuno-transfer method of the present disclosure.

According to a sixth aspect, an Immuno-NLP engineered-cell is described, which is obtained by the Immuno-NLP transfer method for transferring an immune cell transmembrane protein of the disclosure into the immune cell, presenting at least one NLP-transferred immune cell transmembrane protein on the membrane without detectable DNA or RNA encoding for the immunoreceptor protein.

According to a seventh aspect, an Immuno-NLP engineered-cell population is described, which when obtained by the Immuno-NLP transfer method for transferring an immunoreceptor of the disclosure into the immune cell, the Immuno-NLP transferred-cell population comprises a viable cell within the population presenting the immune cell transmembrane protein on their cell membrane without detectable DNA or RNA encoding for an immune cell transmembrane protein within the population. In the Immuno-NLP transferred cell population of the disclosure, a detectable transferred immune cell transmembrane protein, such as the immunoreceptor, such as a CAR protein, can found in >2% of viable cells.

According to an eighth aspect, an Immuno-NLP engineered cell method and a system are described for treating or preventing in an individual a cancer condition, an infectious disease condition, an immune related condition, and/or a fibrotic condition, or conditions associated thereto, the method comprising administering to the individual one or more Immuno-NLP engineered cells of the present disclosure and/or an Immuno-NLP transferred-cell population of the disclosure in an effective amount to elicit an immune response in the individual to the immunoreceptor protein presented on the Immuno-NLP engineered cells or Immuno-NLP engineered population of the disclosure.

The Immuno-NLP engineered cell system comprises one or more Immuno-NLP engineered cells of the present disclosure and/or an Immuno-NLP transferred-cell population of the disclosure together with reagents for cell preparation and use in vitro or in vivo and/or for therapeutic administration for Immuno-NLP cell therapy.

According to a ninth aspect, a pharmaceutical composition is described comprising one or more Immuno-nanolipoprotein particles of the present disclosure, together with a suitable vehicle.

According to a tenth aspect, a composition is described comprising one or more Immuno-NLP engineered cell and/or Immuno-NLP-transferred cell populations of the present disclosure together with a suitable vehicle.

Immuno-nanolipoprotein particles and related cells, compositions, methods and systems, in several embodiments herein described allow loading of various types of transmembrane proteins including transmembrane protein that are particularly insoluble such as CARs and other proteins containing an scFv region.

Immuno-nanolipoprotein particles and related cells, compositions, methods and systems, in several embodiments herein described allow to manufacture NLPs carrying one or more immuno-receptors such as CARs in an amount and configuration allowing nano-delivery of functional CARs on the membrane of immune cells.

Immuno-nanolipoprotein particles, and related cells, compositions, methods and systems, in several embodiments herein described allow to provide immune cells modified to present an immunoreceptor such as a CAR protein which are retargeted to recognize cells without genetic engineering of the immune cells.

Immuno-nanolipoprotein particles and related cells, compositions, methods and systems, in several embodiments herein described allow to perform re-targeting of immune cells by adding immunoreceptors, such as CARs, on the immune cells within a rapid process which can be performed within a time range from 6 hours to 24 hours, expandable to a time range from 1 hour to 1 week.

Immuno-nanolipoprotein particles and related cells, compositions, methods and systems, in several embodiments herein described allow to nano-delivery of immunoreceptor proteins, such as CAR proteins on the immune cells and re-targeting of the related cellular immunity by adding chimeric antigen receptors with an improved survival of immune cells.

Immuno-nanolipoprotein particles and related cells, compositions, methods and systems, in several embodiments herein described provide immune cells with presenting immunoreceptor proteins such as CAR proteins specific for target-cells which have not been genetically modified and therefore have no genomic modifications or vector related toxicity. Viral transduction is known to be associated with genotoxicity, encompassing gene expression dysregulation, aberrant splicing, and epigenetic modifications [1-4], which can result in altered cell behavior or viability. Importantly, previous immune cell engineering techniques relying on genomic insertion of polynucleotide encoding for an immuno-receptor such as a CAR protein have the potential for malignant transformation [5, 6]. In therapeutic treatment where the immunoreceptor is a CAR protein, these risks are possibly related to risk of T-cell malignancies in CAR-T treated patients noted in a recent FDA warning letter [7].

Immuno-nanolipoprotein particles and related cells, compositions, methods and systems, in several embodiments herein described provide immune cells with presenting immunoreceptors such as CAR proteins specific for target-cells which have a finite lifespan of modification. Due to protein recycling, Immuno-NLP transferred immune cells show limited lifetime of CAR on the cell surface, resulting in improved control and potentially lower risk of on-target-off-tumor or off-target toxicity for an individual receiving the retargeted immune cells for example as part of a diagnostic and/or therapeutic treatment.

Immuno-nanolipoprotein particles and related cells, compositions, methods, and systems herein described can be used in connection with various applications wherein a soluble suspension of immunoreceptor protein such as a CAR protein is desired, or where presentation of a functional immunoreceptor such as a CAR protein in an immune cell is desired, or where an immune cell presenting the immunoreceptor in particular when the immunoreceptor is a CAR protein in absence of genetic engineering of the immune cells is desired or where an immune cell with a transferred immunoreceptor and in particular a transferred CAR protein of limited lifespan is desired.

For example, the Immuno-nanolipoprotein particles and related cells, compositions, methods, and systems herein can be used in immune cell therapies, including chimeric antigen T-cell therapy, and stem cell transplants and/or any other application where CAR delivery into immune cells to obtain the related rapid and safe retargeting of immune cells to target specific cells or pathogens, are desired. Another example is provided by application in which CAR delivery into immune cells improves survival of transplanted cells during a stem cell transplant. Another example is rapid design and screening of new CAR designs or new cell modification protocols or in vitro or in vivo study of CAR behavior in immune cells.

Further exemplary applications: cell therapy, chimeric antigen receptor biochemistry, chimeric antigen receptor discovery or development, chimeric antigen receptor T-cell biology, chimeric antigen receptor NK-cell biology, chimeric antigen receptor NK-T-cell biology, chimeric antigen receptor macrophage biology, T-cell receptor transgenic cells, engineered immune effectors. Additional exemplary applications include uses of nanolipoprotein particles in several fields including basic biology research, applied biology, bioengineering, molecular biology, synthetic biology, immune biology, medical research, medical diagnostics, structural biology, therapeutics, vaccine development, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIGS. 1A to 1C, show a schematic representation of the structural features of the immune cell transmembrane proteins of the present disclosure. In particular FIG. 1A shows a schematic representation of a transmembrane protein in the sense of the disclosure. FIG. 1B shows a schematic representation of immunoreceptors in the sense of the disclosure. FIG. 1C is a schematic representation of an exemplary CAR protein in the sense of the disclosure which comprises an antigen recognition domain, a hinge region, a transmembrane domain, an internal signaling and costimulatory domain regions.

FIGS. 2A to 2E provide schematic representation of exemplary first generation CAR proteins (FIG. 2A), second generation CAR proteins (FIG. 2B), third generation CAR proteins (FIG. 2C), fourth generation CAR proteins (FIG. 2D) and fifth generation CAR proteins (FIG. 2E) from Subklewe et al 2019. [8]

FIGS. 3A and 3B show a schematic representation of an exemplary telodendrimer suitable to be included in CAR-t-NLPs herein described. In particular, FIG. 3A shows a schematic representation of exemplary Cys-telodendrimer suitable to be included in CAR-t-NLPs herein described. FIG. 3B shows a schematic representation of exemplary His-telodendrimer suitable to be included in CAR-t-NLPs herein described.

FIGS. 4A and 4B show sequences of scaffold protein that can be used to provide CAR-NLPs herein described. FIG. 4A shows codon optimized nucleotide (SEQ ID NO: 1) and amino acid sequence (SEQ ID NO: 2) for LLNL Mouse Δ49ApoA1. FIG. 4B shows codon optimized nucleotide (SEQ ID NO: 3) and amino acid sequence (SEQ ID NO: 4) for LLNL Mouse ApoE4, 22k.

FIG. 5 shows a wild-type mouse nucleic acid sequence (SEQ ID NO: 5) for the encoded Δ49ApoA1 gene.

FIG. 6 shows LLNL codon optimized mouse nucleic acid sequence (SEQ ID NO: 6) for the encoded Δ49ApoA1 gene.

FIG. 7 shows a LLNL codon optimized BALBC mouse nucleic acid sequence (SEQ ID NO: 7) for the encoded Δ49ApoA1 gene.

FIG. 8 shows that synthesis of CAR-NLP using previously published methods generates fails to generate soluble CAR-NLP suspension. CAR-NLP were synthesized using cell-free techniques described in previous work then CAR-NLP(lane 1) and empty NLP(lane 2) were analyzed by PAGE and total protein staining along with western blotting for FLAG (Element of CAR) and His (Element of ApoA1). We find that these methods generate 8.9 CAR/100 NLPs, with <20% solubility.

FIGS. 9A to 9E show poor uptake of beta 2 adrenergic receptor-NLP into human peripheral blood T-cells using the methods described in Patriarchi et al. GFP tagged beta 2 adrenergic receptor (B2AR)-NLPs were constructed using cell-free expression using the methods described in Patriarchi et al. T-cells were isolated, frozen, thawed and cultured in RPMI-1640 medium T-cell specific medium then dosed for 24 hours with: FIG. 9A-vehicle (no NLP added), FIG. 9B-5 μg/ml control NLPs (lipid+scaffold protein only), FIG. 9C-5 μg/ml B2AR-NLP, FIG. 9D-100 μg/ml B2AR-NLP, or FIG. 9E-100 μg/ml B2AR-NLP in the presence of 3 μg/ml anti-CD3 and 5 μg/ml anti-CD28 activation reagents, followed by flow cytometry analysis on viable cells. Data displayed shows GFP intensity on the horizontal axis and side scatter area on the vertical axis. Detectable uptake of GFP is not observed in the 5 μg/ml conditions used in previous work. Observable uptake is seen only in the 100 μg/ml B2AR-NLP conditions (FIG. 9D and FIG. 9E) and efficient uptake requires activation reagent treatment.

FIG. 10 shows that activated T-cells are resistant to NLP mediate protein uptake using the methods described in Patriarchi et al. Human peripheral blood T-cells and 4t1 tumor cells were dosed with between 0 and 200 μg/ml GFP-labeled B2AR-NLP then analyzed by flow cytometry to determine percentage of GFP+ cells in the viable cell fraction. Robust, dose dependent uptake of B2AR was observed in 4T1 cells, with a maximum of >70% of cells displaying GFP, however huPBMC T-cells treated similarly showed a significantly worse uptake, with essentially undetectable uptake for any dose less than 200 μg/ml. Reaching the 70% uptake possible with 4T1 cells would require dosing T-cells with >700 μg/ml (˜10 micromolar) protein, which is a significant deviation from past work.

FIGS. 11A to 11D show T-cell damage with increasing doses of CAR-NLP, when that material is formulated as in previous work. Human PBMC T-cells were activated using antiCD3/CD28 reagents and dosed with between 4 and 100 μg/ml dose of CAR-NLP for 1 day. FIG. 11A: at 4 μg/ml dose used in previous work (Patriarchi et al) and at 10 μg/ml dose T-cells form clusters. At 40 μg/ml (FIG. 11C) and 100 μg/ml (FIG. 11D), clusters do not form indicating disruption of T-cell behavior.

FIG. 12 shows increasing doses of CAR-NLP formulated as in [9] can cause cytotoxicity. Human PBMC T-cells were activated using antiCD3/CD28 reagents and dosed with between 0 and 100 μg/ml dose of CAR-NLP for 1 day. Decreasing viability is observed with increased CAR-NLP dose.

FIG. 13 shows that CAR-NLP solubility issues can be alleviated by reducing the temperature of expression. CAR-NLP were synthesized using cell-free expression at different temperatures from 15 to 30° C. then total and soluble fraction were analyzed for each temperature. Solubility at 30° C. was essentially 0, whereas >80% of CAR protein was soluble when synthesized at 20° C. FIG. 13 shows that formulation can improve uptake into T-cells. By careful removal of imidazole and insoluble protein (through cell-free expression at low temperature) detectable uptake of CAR can be found at high NLP doses.

FIG. 14 reports a graph showing T cell viability following treatment with increasing concentrations of CAR-NLP, shows that altering formulation can improve viability issues for human peripheral blood T-cells. By careful removal of imidazole and insoluble protein (through cell-free expression at low temperature) minimal (<10%) changes in cell viability can be observed.

FIG. 15A shows a chart reporting CAR protein uptake in T cells treated with increasing doses of CAR-NLPs. Human peripheral blood mononuclear cells (huPBMCs) were incubated with CAR-bearing nanolipoprotein particles (CAR-NLPs) at indicated concentrations (0-250 μg/mL) for 24 hours and CAR uptake after washing was assessed by flow cytometry using an anti-FMC63-FITC antibody and FACS analysis. Increasing mean free intensity of CAR with increasing dose of CAR-NLP indicates a dose responsive uptake of CAR proteins into T cells.

FIG. 15B shows a chart reporting the functionality of CAR protein delivered into T cells following treatment with CAR-NLPs. T cells were incubated with increasing concentrations of CAR-NLPs, and ability of the chimeric antigen receptor (CAR) on the surface of these cells to bind CD19, the ligand of the CAR was assessed using a CD19 protein-FITC conjugate and FACS. Mean fluorescence intensity (MFI) values were plotted against CAR-NLP concentration, demonstrating a dose-dependent increase in CD19 binding with increasing doses of CAR-NLPs.

FIGS. 15C and 15D show that T-cells treated with CAR-NLP formulated to remove imidazole and insoluble protein can induce T-cells to recognize and kill tumor cells. T-cells were treated with CAR-NLP at doses between 0 and 200 μg/ml overnight in the presence of anti-CD3/CD28 activation reagents, then washed and mixed with Raji tumor cells at 1:1 ratio. After 1 day (FIG. 15C) and 3 days (FIG. 15D), fraction of Raji from total cells was analyzed by flow cytometry. Decreasing fraction of Raji in the total viable cell fraction indicates antitumor cytotoxicity.

FIG. 16 shows that conjugating an scFv to an NLP permits efficient delivery to T cells, whereas free scFv in the absence of NLP components is not taken up into T cells.

FIGS. 17A to 17F report result of experiments showing that CAR-NLP engineering can be accomplished using the methods of the present disclosure in both CD4+ and CD8+ T cells, B cells, and NK cells.

FIG. 18 shows a chart illustrating the effect of low temperature synthesis reduces total yield but increases soluble yield for beta-2 adrenergic receptor containing NLPs. B2AR-NLPs were synthesized using methods in Patriarchi et al. except with changing temperature from 30° C. (used in Patriarchi et al.) to 20° C. and including bodipy labeled lysine to identified synthesized proteins. For soluble protein fraction, materials were centrifuged at max speed in a tabletop centrifuge and the supernatant was collected. Total protein and soluble protein were analyzed by SDS-PAGE and band density was measured and compared.

FIG. 19 shows a chart illustrating the relationship between synthesis temperature and cargo protein loading in the total and soluble protein fractions. The ratio of beta-2 adrenergic receptors per NLP is higher in the NLPs synthesized at 30° C. compared to at 20° C., however this material is poorly soluble. Removing the insoluble fraction reveals that the cargo loading in the 20° C. synthesis is >2-fold higher than in the 30° C. synthesis at all timepoints investigated.

FIG. 20 shows a picture and a diagram reporting the results Cell Free expression of CAR protein. FIG. 20 Panel A shows a SDS PAGE of yield from cell free synthesis of CAR protein. FIG. 20 Panel B shows a Band densitometry of synthesized CAR protein and soluble fraction. While protein synthesis could be detected, soluble yield was extremely poor, and almost indistinguishable from background

FIG. 21 shows pictures and charts reporting screening data for optimizing CAR-NLP solubility and yield. FIG. 21 Panel A: Methods for cell free co-expression of CAR and Apolipoprotein in the presence of lipids resulting in synthesis of CAR-NLPs. FIG. 21 Panel B: Representative SDS page for lipid screening for CAR-NLPs. FIG. 21 Panel C: band densitometry for total and soluble CAR NLP synthesis (n=2 replicate experiments), with GFP as a control. Lipid composition affected total yield and soluble yield. FIG. 21 Panel D: % soluble CAR from total protein. GFP serves as a representative of a soluble protein. DMPC and EggPC represent the most soluble lipid compositions. FIG. 21 Panel E: representative SDS-PAGE for reaction temperature with DMPC at 18 hours of synthesis time. FIG. 21 Panel F: quantification of total and soluble yield by reaction temperature. While synthesis at 30-C resulted in the highest total yield, the solubility of this material was essentially 0. FIG. 21 Panel G: Soluble fraction of total protein yield. Solubility peaked at 20 degrees C. FIG. 21 Panel H: Representative SDS-PAGE for yield by reaction time for DMPC at 20 degrees C. FIG. 21 Panel I: quantification of reaction yield. Total and soluble yield peak at around 8 hours and decrease over time. FIG. 21 Panel J: Soluble yield declines after 24 hours.

FIG. 22 shows pictures and charts illustrating data concerning the characterization of CAR plasmid expression and codon optimization. FIG. 22 Panel A shows SDS-PAGE analysis of in vitro translation products from mammalian cell-free lysates transfected with different CAR plasmid constructs. Distinct bands corresponding to the CAR protein are visible for each construct. . . . FIG. 22 Panel B shows Bar graph comparing protein expression from codon-optimized versus non-optimized CAR plasmids, showing enhanced expression in codon-optimized constructs. FIG. 22 Panel C shows Quantification of codon optimization effects on CAR protein yield, based on fluorescence intensity or band densitometry, confirming improved translational efficiency with codon-optimized sequences.

FIG. 23 shows pictures and charts reporting results of an exemplary synthesis and qualification of CAR-NLPs. FIG. 23 Panel A: Quantitative SDS-PAGE showing synthesis of CAR-NLPs using either a commercial cell free lysate kit (R-CF) or a homemade cell free lysate (H-CF). FIG. 23 Panel B: Quantification of yield of CAR and APO proteins reveals a 0.75 CAR: NLP ratio (2 Apolipoproteins/NLP) FIG. 23 Panel C: Western blot showing positive staining for FLAG tag in the CAR protein. FIG. 23 Panel D: Western blot showing positive staining for HIS tag on Apolipoprotein. FIG. 23 Panel E: Dot blot showing binding of a conformation sensitive anti-FMC63 antibody against the scfv of the CAR. FIG. 23 Panel F: Positive binding of CAR protein to cells. Magnetic anti-his beads were conjugated with either: His tagged FMC63 antibody, CAR-NLP(using the His tag on the apolipoprotein) or empty NLP. These beads were mixed with CD19+Raji cells, then washed to remove loose beads and pulled down using a magnet. FMC63 or CAR-NLP treated beads decorated the surface of Raji cells and FIG. 23 Panel G: pulled down the majority of cells, whereas empty NLPs pulled down less than half. FIG. 23 Panel H: More beads per cell were observed in the CD19 antibody or CAR-NLP conditions than with the Empty NLP.

### DETAILED DESCRIPTION

Provided herein are Immuno-nanolipoprotein particles and related cells compositions methods and systems, configured to allow engineering of immune cells by transfer of immune-transmembrane proteins onto the membrane of the immune cells in functional form and without the need of genetic engineering of the immune cells.

The term “transmembrane protein” or “membrane protein” or “integral membrane protein” as used in the present disclosure indicate a protein including at least one transmembrane domain (TMD) or TM which indicates any protein segment which is thermodynamically stable in a membrane, as will be understood by a skilled person.

The structure of immune cell transmembrane proteins is essential for their function in cellular communication, signal transduction, and immune responses. In this respect three domains can be identified an extracellular domain, an intracellular domain and a transmembrane domain (see Example 1).

The transmembrane domain is the core feature of transmembrane proteins which provides their ability to integrate into the lipid bilayer of the cell membrane and has important effect on the protein functionality as will be understood by a skilled person. Transmembrane proteins have a unique orientation in the membrane, with distinct cytosolic (inside the cell) and non-cytosolic (outside the cell) domains. This orientation is crucial for their function, as it determines how they interact with other cellular components and signaling molecules

Transmembrane segments of transmembrane proteins typically form a hydrophobic structure, such as an α-helical structure or a β-sheet. These conformations within the protein segment allow hydrogen bonding to stabilize the protein structure and expose hydrophobic residues to the lipid rich environment within the membrane bilayer, stabilizing the protein within the hydrophobic environment of the lipid bilayer [10].

Some transmembrane proteins are single-pass transmembrane proteins which cross the membrane only once with a single α-helix. An example is glycophorin, which functions primarily

in cell-cell recognition and signaling [10]. Some transmembrane proteins are multi-pass transmembrane proteins which have multiple transmembrane segments, often forming complex structures. Examples include G-protein-coupled receptors (GPCRs) and ion channels, which are critical for signal transduction and transport of ions and molecules across the membrane [10].

Transmembrane proteins play a pivotal role in transmitting signals from the extracellular environment to the intracellular machinery. This is achieved through conformational changes upon ligand binding, which then trigger intracellular signaling pathways [11, 12]. The fluid nature of the lipid bilayer allows transmembrane proteins to diffuse laterally within the membrane, facilitating interactions and the formation of signaling complexes as will be understood by a skilled person

Immune cell transmembrane proteins are integral to the function of the immune system. Their structure, characterized by transmembrane domains, allows them to interact with the lipid bilayer and other proteins, enabling them to perform their roles effectively.

Transmembrane proteins play several crucial roles in cellular physiology enabling the cell to detect, respond to, and interact with their environment effectively. [10] For example, transmembrane proteins can function as channels or transporters that facilitate the movement of ions, nutrients, and other molecules across the cell membrane. This transport is essential for maintaining cellular homeostasis and regulating metabolic processes [13, 14]. Transmembrane proteins can also contribute to the structural integrity of the cell membrane. They help anchor a cell membrane to the cytoskeleton, maintaining the cell's shape and stability [10]. Transmembrane proteins can have enzymatic functions, catalyzing biochemical reactions at the membrane surface. This can include processes like phosphorylation, which is important in signal transduction pathways [10, 14]. Certain transmembrane proteins are involved in cell adhesion, helping cells stick to each other and to the extracellular matrix. This is important for tissue formation and immune cell interactions [10]. Furthermore, transmembrane proteins can also act as receptors that detect extracellular signals and transmit them into the cell. This signal transduction is crucial for cellular communication, allowing cells to respond to their environment and coordinate immune responses. Examples include G protein-coupled receptors (GPCRs) and receptor tyrosine kinases [13] (see Example 1).

In immune cells, transmembrane proteins can be involved in recognizing pathogens, presenting antigens, and initiating immune responses. For example, in immune cells, transmembrane proteins are essential for: antigen recognition, co-stimulation and inhibition, cytokine binding and binding of the immune cells with to other cells and tissues aiding in immune surveillance and response.

Immune cell transmembrane proteins (ICTMs) include a diverse class of membrane-anchored signaling and recognition molecules that are involved in immune function. Structurally, ICTMs can be broadly classified into single-pass ICTMs, which possess a single helical transmembrane domain flanked by an extracellular ligand-binding domain and an intracellular signaling or adaptor domain, and multi-pass ICTMs, which span the membrane multiple times and typically form helical bundles that support ligand binding and signal transduction. Examples of single-pass ICTMs include CD19, PD-1, CD4, and chimeric antigen receptors (CARs), while multi-pass ICTMs include G-protein coupled receptors (GPCRs), cytokine receptors with shared subunits, and ion channels.

Single-pass ICTMs frequently contain flexible or disordered domains and lack the compact, self-stabilizing architecture seen in many multi-pass proteins. The absence of multiple membrane-spanning regions reduces conformational constraints, resulting in greater dynamic motion and an increased tendency toward aggregation or misfolding in vitro. This structural simplicity also complicates stabilization and purification efforts, particularly in detergent-free systems or when attempting to reconstitute the protein into artificial membranes or nanodiscs, making single-pass ICTMs more difficult to manage than multi-pass ICTM proteins as will be understood by a skilled person.

Exemplary single pass ICTM, comprise transmembrane proteins CD19 (a marker for B cells, involved in B cell activation and development), CD20 (another B cell marker, targeted by certain therapeutic antibodies), CD45 (a tyrosine phosphatase found on all nucleated hematopoietic cells, important for T and B cell receptor signaling), CD56 (a marker for natural killer (NK) cells and a subset of T cells), CD80 (B7-1)(a co-stimulatory molecule found on antigen-presenting cells, interacts with CD28 and CTLA-4 on T cells), CD86 (B7-2)(another co-stimulatory molecule on antigen-presenting cells, which also interacts with CD28 and CTLA-4), and other transmembrane proteins identifiable by a skilled person.

Additional, single pass ICTM transmembrane proteins of immune cells comprise also immunoreceptors which are a transmembrane protein located on the surface of immune cells that bind to specific ligands, such as antigens, antibodies, or other signaling molecules, and initiate a cascade of intracellular signaling events that regulate immune cell activity (see Example 1).

Exemplary single pass immunoreceptors ICTM comprise CD4, CD8, cytokine receptors such as Interleukin-2 receptor (IL-2R): Interleukin-6 receptor (IL-6R); and Tumor necrosis factor receptor (TNFR), checkpoint inhibitors, such as PD-1 (Programmed Cell Death Protein 1) and additional receptor identifiable by a skilled person.

Immunoreceptors in the sense of the disclosure also comprise engineered molecules such as Chimeric antigen receptors (see Example 1 and Example 2).

Chimeric antigen receptors (CARs) are proteins that are transgenically expressed in cells of the immune system to alter adhesion of those immune cells to a targeted cell or pathogen in a manner that can weaken, damage and/or induce death in the targeted cell or pathogen.

The term “chimeric antigen receptor” or “CAR” as used herein indicates a synthetic receptor that combines components from different source proteins into a single protein, designed to provide immune cells with the ability to recognize and attack specific antigens on target-cells. In particular, CARs are artificial constructs that combine segments from different sources to create a new entity with unique properties. For example, CARs encompass proteins engineered by splicing together genetic material to produce a hybrid protein that can recognize cancer-associated antigens with high specificity. This engineered receptor is then expressed on the surface of T-cells, enabling them to identify and kill cancer cells that express the target antigen [15, 16] or to recognize and eliminate pathogens, or to eliminate or suppress unwanted cell growth and activation found in autoimmune conditions such as lupus or multiple sclerosis or graft vs. host disease, e.g. transplant rejection [17] or to treat fibrotic disease or fibrosis associated with heart or lung disease [18], or to remove senescent-cells [19].

Accordingly, “chimeric antigen receptor” as herein described comprise synthetic proteins designed to artificially recreate the ability of T-cells to recognize, activate, and attack target-cells such as cancer cells. These CAR proteins are engineered by combining different domains that each play a role in the activation and targeting capabilities of the T-cells. The structure of CARs is modular, consisting of several key components, each contributing to the receptor's overall function as will be understood by a skilled person (see Example 2).

In particular, structural components of CAR protein comprises:

1. An Antigen Recognition Domain (herein also CAR-ARD), which is a protein structure forming part of the extracellular regions of the CAR protein and including domains conferring to the CAR an ability to recognize specific target protein such as antigens on the surface of a target-cell such as a cancer cells identifiable by a skilled person. Typically, an ARD comprises a single chain antibody fragment, e.g., an scFv, a single chain camelid antibody, e.g., a VHH or nanobody or DARPin, or a protein which naturally binds to a desired ligand, but can also encompass combinations of 2 or more of the above elements, e.g., bispecific or multi-specific constructs, connected to the same CAR structure or used in concert with each other.

2. A Transmembrane Domain (herein also CAR-TMD), which is a hydrophobic protein or lipid structure configured to anchor the CAR protein to a cell membrane, which is identifiable by a skilled person. In particular, a transmembrane domain typically comprises at least one hydrophobic alpha helix configured to span or integrate into the lipid bilayer of a cell membrane, anchoring the CAR to the cell membrane, as exemplified by the transmembrane domain from human CD28. The alpha-helix is a common structural motif in transmembrane proteins due to its ability to stabilize hydrophobic interactions within the membrane's lipid environment, as will be understood by a skilled person. The transmembrane domain can also be understood to comprise lipid or lipophilic moieties, such as lipids with modified head groups to enable binding and anchorage of an extracellular protein antigen recognition domain.

3. Optionally, intracellular Signaling Domains (herein also CAR-ISD), which are a protein structure motifs configured to activate immune cells presenting the CAR upon antigen recognition by the antigen recognition domain of the CAR protein, for example by allowing phosphorylation of tyrosine residues. Examples include CD3ζ domain, CD8a 4-1BB, CD40, IL15RA and CTLA4 intracellular domains, combinations thereof or other protein domains capable of inducing a activating response in immune cells.

In particular, in embodiments herein described a CAR-ADR can comprises various structure enabling an engineered immune cell to recognize and bind to specific antigens on the surface of target cells or pathogens.

An exemplary structure that can form a CAR-ADR in the sense of the disclosure is a Single-Chain Variable Fragment (scFv), the most common antigen-binding motif used in CARs. ScFvs are fusion proteins derived from the variable regions of heavy (VH) and light (VL) chains of immunoglobulins, linked together by a flexible peptide linker as will be understood by a skilled person. This design allows for the retention of the antigen-binding specificity of the antibody from which it is derived. ScFvs are favored for their case of genetic encoding and ability to be derived from existing monoclonal antibodies, providing a common scaffold to target a wide range of

desired antigens [21]. Combinations of scFvs have been used as well to create CAR-ARDs with sensitivity to multiple epitopes or multiple proteins.

Another exemplary structure that can form a CAR-ADR in the sense of the disclosure is provided by single chain antibodies found naturally in camelids and other species such as sharks which are formed by the variable domains of heavy-chain-only antibodies also known as VHH domains or nanobodies. Nanobodies are the smallest fragment capable of antigen binding, comparable to conventional antibodies in terms of affinity and specificity. Nanobodies offer several advantages, including small size, high solubility and stability, low immunogenicity, and high tissue penetration. Their compact size and lack of a variable light chain make them particularly suitable for targeting epitopes that are difficult to access with larger molecules like scFvs

A further exemplary structure that can a CAR-ARD in the sense of the disclosure is provided by DARPins. Designed Ankyrin Repeat Proteins (DARPins) are another alternative to scFvs for the antigen recognition domain. DARPins are small, highly stable proteins that can be engineered to bind with high specificity and affinity to a wide range of target antigens. Their modular structure allows for the precise control over binding properties, making them a versatile tool for CAR design [22].

Another exemplary structure that can form a CAR-ARD in the sense of the disclosure is provided by Natural Ligands and Receptor-Based Domains. Utilizing natural ligand and receptor-based domains for antigen recognition represents a different approach. This strategy exploits the natural receptor-ligand interactions found in physiology, where the specificity of the binder domain is defined solely by its relationship to the native binding partner. Such domains are derived directly from natural human protein elements, posing a low risk of immunogenicity and often retaining the stability of the original protein structure [22]. Natural ligand-receptor pairs are pairs of cell surface protein molecules or soluble molecules found in/on cell types capable of physical interactions and are typically involved in cell-cell recognition, signaling or adhesion. Databases of known receptor-ligand pairs in human biology have been published [23, 24].

A further exemplary structure that can form a CAR-ARD in the sense of the disclosure is provided by peptide-scFv Constructs. Recent developments have also explored the use of peptide-scFv constructs for antigen recognition. These constructs involve the integration of peptides with scFvs to create bi- or multispecific CARs, where linker length and flexibility can significantly influence functionality. This approach allows for the targeting of multiple antigens or epitopes by the same protein, potentially enhancing the specificity and efficacy of CAR T-cell therapies

The Antigen Recognition Domain of a CAR protein in the sense of the disclosure is therefore usually composed of one or more small protein structures (<30 kDa) expressed in a single chain that provides antigen specificity, defined as a significantly higher affinity for a desired protein ligand than for an unrelated protein. Accordingly, ADR have a protein structure that is characterized by the ability to bind to a given ligand with high affinity relative to unrelated proteins as will be understood by a skilled person.

Different antigen recognition domain structures offer distinct advantages for CAR design, as will be understood by a skilled person from the versatility and specificity of scFvs and nanobodies to the low immunogenicity and natural stability of ligand/receptor-based domains. The choice of antigen recognition domain is functional to the functionality of the CAR protein following delivery to an immune cells, influencing both the targeting accuracy and the overall outcome of the cell targeting as will be understood by a skilled person

In embodiments herein described a transmembrane domain (TMD) of a chimeric antigen receptor (CAR) protein in the sense of the disclosure comprises different structure configured to anchor the CAR to a target-cell membrane, which can influence the function, stability, and signaling of CAR-T-cells. Various structures derived from natural proteins have been utilized to form the TMD in CAR constructs, each offering unique properties that can affect the overall performance of CAR-T-cell therapy [28].

In particular, a CAR-TMD in the sense of the disclosure is provided by the transmembrane domain of the human CD3 chain. CD247/CD3ζ (and homologous sequences from other species) represents a protein component of the T-cell receptor (TCR) complex and its transmembrane domain (amino acids 31-51 plus potentially flanking regions, structurally an alpha helix) has been used as a transmembrane domain in CAR constructs. It may facilitate CAR-mediated T-cell activation by mediating CAR dimerization and incorporation into endogenous TCRs. However, using the CD33 transmembrane domain might come at the cost of decreasing CAR stability compared to other options [28].

Another exemplary CAR-TMD in the sense of the disclosure is provided by the transmembrane domain of human CD4 protein (and homologous sequences from other species). It is primarily known as a co-receptor involved in the immune response, particularly in the activation of T-cells. CD4 is another protein that has been used to provide the transmembrane domain (amino acids (AAs) 397-418) plus potentially flanking regions, structurally also an alpha helix in CAR constructs. The impact of the CD4 transmembrane domain on CAR function is not as well studied as others [28, 30].

A further exemplary CAR-TMD in the sense of the disclosure is provided by the transmembrane domain of CD8a protein from humans and homologous sequences from other species. The CD8a protein transmembrane domain (defined as protein sequences with >50% similarity to CD8a amino acids AAs 183-203, comprised structurally of an alpha helix) is frequently used in CAR designs. It is derived from the CD8a molecule, which acts as a co-receptor for the TCR. CARs with the CD8a transmembrane and hinge domains have been reported to release decreased amounts of TNF and IFNγ and have decreased susceptibility to activation-induced cell death (AICD) compared to CARs with these domains derived from CD28 [28].

A further exemplary CAR-TMD in the sense of the disclosure is provided by a transmembrane domain of CD28 protein from humans or homologous sequences from other species. The CD28 transmembrane domain (AAs 153-179, structurally an alpha helix) is commonly used in CAR constructs and is known for enhancing CAR expression and stability. CD28 is a critical costimulatory molecule in T-cells, and its transmembrane domain has been associated with improved CAR-T-cell function and persistence [28, 31].

A further exemplar of CAR-TMD includes lipids incorporated within the nanodisc which can anchor an ARD domain. Lipid domains can comprise hydrophobic posttranslational modifications of proteins such as palmitoylation or myristoylation, or functionalized lipids that can bind a protein hinge/ABD region, either of which results in an anchorage of an ARD to a NLP.

The Transmembrane Domain of a CAR protein in the sense of the disclosure is therefore usually composed of hydrophobic protein domains or lipids which incorporate into the NLP by basis of hydrophobicity, providing the function of membrane anchorage. Transmembrane protein domains in mammalian transmembrane proteins typically contain alpha helical or beta barrel secondary structures that provide hydrophobicity. These domains can either span or integrate into the plasma membrane of a cell. Lipid domains can arise from palmitoylation, myristoylation or other hydrophobic posttranslational modifications that promote protein association with a membrane, or from functionalized lipids that can bind a protein hinge/ARD region.

The transmembrane domain, while primarily serving to anchor the CAR to the T-cell membrane, also influences CAR expression level and distribution within a cell, and CAR stability, and can be active in signaling or synapse formation. The choice of transmembrane domain (e.g., CD3ζ, CD28) can impact CAR function and T-cell cytokine production [28].

In embodiments herein described, transmembrane domains that can form part of CAR-TMD are chosen based on their ability to effectively anchor the CAR in the T-cell membrane and their influence on the signaling and functional outcomes of the CAR-T-cells. The selection of a specific transmembrane domain can be based on the desired properties of the CAR-T-cell, such as stability, expression level, and the ability to activate and proliferate in response to antigen recognition [28, 30, 31].

The intracellular signaling domains of CAR proteins in the sense of the disclosure are optional components that transmit activation signals within the T-cells upon antigen recognition. It can be formed by zero, one, or more signaling domains as it would be understood a by a skilled person.

An exemplary CAR-ISD in the sense of the disclosure is provided by the cytoplasmic tail of a CD3ζ chain. The CD3ζ cytoplasmic tail (defined as protein sequences with 50% similarity to CD3ζ amino acids 52-164) is a critical component of the T-cell receptor (TCR) complex and is commonly used in CAR design for its role in signal transduction. It contains Immunoreceptor Tyrosine-based Activation Motifs (ITAMs)(comprised of a tyrosine separated from a leucine or isoleucine by 4 amino acids, commonly expressed as YxxL/I) which are essential for initiating T-cell activation and downstream signaling pathways.

Another exemplary CAR-ISD in the sense of the disclosure is provided by the intracellular domain of the CD28 protein (defined as protein sequences with >50% similarity to CD28 amino acids 180-220). CD28 is another frequently used co-stimulatory domain in CAR design. The intracellular domain of CD28, containing motifs like YMNM and proline-rich regions (e.g. PYAP), It enhances T-cell activation, providing a stronger and more sustained signal compared to CD3ζ alone. The CD28 domain contributes to the survival and proliferation of T-cells.

A further exemplary CAR-ISD in the sense of the disclosure is provided by the cytoplasmic/intracellular domain of 4-1BB(CD137 or Tumor necrosis factor receptor superfamily member 9)(defined as protein sequences with >50% similarity to 4-1BB amino acids 214-255). 4-1BB is a co-stimulatory molecule that, when its cytoplasmic domains are included in CAR constructs, has been shown to improve the persistence and efficacy of CAR T-cells. It supports the survival and long-term function of T-cells in the harsh tumor microenvironment

An additional exemplary CAR-ISD in the sense of the disclosure is provided by the cytoplasmic/intracellular domain of OX40 (CD134/TNFRSF4)(defined as protein sequences with >50% similarity to OX40 amino acids 236-277). OX40 is another co-stimulatory receptor used in some CAR designs to enhance the immune response. It works by promoting T-cell proliferation and survival, similar to 4-1BB.

A further exemplary CAR-ISD in the sense of the disclosure is provided by the intracellular signaling domain of ICOS (Inducible T-cell CO-Stimulator/CD278). The intracellular signaling domain of ICOS (defined as protein sequences with >50% similarity to ICOS amino acids 162-199) includes a YMFM motif, is incorporated into some newer CAR designs to further enhance T-cell function. It is known to support T-cell expansion and survival, providing a potent co-stimulatory signal that complements signals from CD28 and 4-1BB [35].

The Intracellular Signaling Domain of a CAR protein in the sense of the disclosure is therefore composed of the cytoplasmic domain of a protein involved in immune cell activation and expansion. Many of these proteins in T-cells are either members of the immunoglobin super family or the TNF receptor family. Common motifs include YxxL/I, YMFM and proline rich domains that can activate signaling through PI3K, AKT, ERK NFκB, or NFAT pathways.

In some embodiments, a CAR protein in the sense of the disclosure can further comprise:
4. A Hinge Region (herein also CAR-HGR), which is a protein structure that connects the antigen recognition domain to the transmembrane domain, and it is configured to provide flexibility and space, allowing the antigen recognition domain to effectively engage with the antigens on target-cells as will be understood by a skilled person, and
5. A Costimulatory Domains (herein also CAR-CSD), which are domains configure to enhance the efficacy, persistence, and overall function of CAR T-cells as will be understood by a skilled person.

In particular, the CAR-HGR serves as a crucial structural component of the CAR protein that connects the extracellular antigen recognition domain to the transmembrane domain. This region provides flexibility and extends the antigen recognition domain away from the T-cell surface, facilitating effective interaction with target antigens on cancer cells. The composition and length of the hinge region can significantly influence the function and efficacy of CAR T-cells, affecting aspects such as antigen binding, CAR expression, and signal transduction. Various structures derived from different sources can form the hinge region in CAR proteins, each offering distinct properties [28].

An exemplary structure that can form a CAR-HGR in the sense of the disclosure is provided by the membrane-proximal stalk region up of CD8a (AAs 128-182) to the conserved cysteine necessary for the CD8a Ig loop. CD8a, hinge region derived is one of the hinge regions commonly used structures in CAR design. Due to its intrinsically disordered protein structure, it provides a certain level of flexibility and extension, facilitating the antigen recognition domain's access to target antigens [30].

Another exemplary structure that can form CAR-HGR in the sense of the disclosure is provided by the membrane-proximal stalk region of CD28 up to its Ig-like V-type domain (Short CD28 Hinge) or portion of the Ig-like V-type domain of CD28 up to the membrane-proximal cysteine residue responsible for intrachain disulfide bonding (CD28-Hinge). This structure not only contributes to the spatial orientation of the antigen recognition domain but may also influence the signaling properties of the CAR, given CD28's role in costimulatory signaling [30].

A further exemplary structure that can form a CAR-HGR in the sense of the disclosure is provided by the hinge region of IgGI (defined as protein sequences with >50% homology to IgGI amino acids 216-231). The hinge region can also be derived from the IgG1 antibody subclass, is typically composed of 15 amino acids and is very flexible. This structure is particularly notable for its ability to extend the antigen recognition domain and its potential interaction with Fcγ receptors. However, IgG-derived spacers, including those from IgG1, can lead to CAR-T-cell exhaustion due to their interaction with Fcγ receptors, which reduces their persistence in vivo. Some strategies attempt to mitigate this issue by mutating IgG Fc receptor binding sites.

An additional exemplary structure that can form a CAR-HGR in the sense of the disclosure is provided by the hinge region of IgG4 which can comprise 12 amino acids: Similar to IgG1, the IgG4 subclass can also provide a hinge region for CAR proteins. IgG4-based hinges are shorter than that of IgG1. It provides intermediate flexibility and is used to reduce interactions with Fcγ receptors, thereby minimizing off-target effects used for their flexibility and extension capabilities. Like IgG1, they may interact with Fcγ receptors, and modifications are sometimes necessary to prevent undesired effects.

Another exemplary protein structure that can form a CAR-HGR in the sense of the disclosure is provided by Non-IgG Based hinges and comprise hinges derived from other T-cell co-receptors or molecules, such as CD35, which are designed to provide specific structural and functional properties to the CAR. Besides the commonly used IgG-based spacers, non-IgG based components naturally expressed on T-cells can also serve as sources for the hinge region. These alternatives might offer different properties in terms of flexibility, length, and potential immunogenicity, although specific examples beyond CD8a and CD28 are not detailed in the provided sources [21].

The choice of hinge region structure is important in the design of CAR protein in the sense of the disclosure, as it can affect the ability of an immune cells following delivery to recognize and engage target-cells effectively, as well as influence the overall therapeutic efficacy and safety profile of the CAR immune cell therapy.

The hinge regions of a CAR protein in the sense of the disclosure are therefore usually composed of flexible protein linkers arising from weakly structured or intrinsically disordered protein regions.

Co-stimulatory domains of a chimeric antigen receptor (CAR) protein in the sense of the disclosure comprise different structure configured to enhance the efficacy, persistence, and overall function of CAR T-cells. Co-stimulatory domains of a chimeric antigen receptor (CAR) protein provide the CAR engineered immune cell with robust-cell expansion, function, and antitumor activity.

An exemplary protein structure that can form a CAR-CSD in the sense of the disclosure is provided in the intracellular domain of the CD28 protein. CD28 is one of the first costimulatory domains utilized in CAR design, effective in providing robust costimulation. It enhances T-cell proliferation, IL-2 production, and is essential for optimal surface expression of CARs. CD28-based CARs can drive T-cells towards exhaustion due to chronic stimulation but are rapid in onset of T-cell activity [36, 37]. The positioning of the CD28 domain within the CAR construct, especially when placed in the membrane-proximal position, significantly influences T-cell activity [36].

Another exemplary protein structure that can form a CAR-CSD in the sense of the disclosure is provided by cytoplasmic/intracellular domain of 4-1BB(CD137). The cytoplasmic/intracellular domain of 4-1BB(CD137) is another widely used costimulatory domain in CAR constructs, known for improving the persistence and efficacy of CAR T-cells. It supports T-cell survival and function in the tumor microenvironment. 4-1BB-based CARs direct T-cells into a novel state of dysfunction, distinct from exhaustion, which can be mitigated by suppressing FOXO3 [36, 38, 39]. There have been challenges with the transduction efficiency and gradual downregulation of the 4-1BBC CAR during the manufacturing process [38].

A further exemplary protein structure that can form a CAR-CSD in the sense of the disclosure is provided by the intracellular signaling domain of Inducible T-cell CO-Stimulator (ICOS). ICOS is another Ig superfamily member assessed for its costimulatory potential in CAR designs. It plays a role in enhancing T-cell function, although specific details on its impact compared to CD28 and 4-1BB were not directly provided in the sources [36].

An additional exemplary protein structure that can form a CAR-CSD in the sense of the disclosure is provided by the cytoplasmic/intracellular domain of OX40 (CD134) or the intracellular (cytoplasmic) domain of CD27. Both OX40 and CD27, members of the TNFR superfamily, have been explored as costimulatory domains in CAR constructs. They are part of the expanded range of costimulatory domains assessed for CAR T-cell activity and proliferation, although specific outcomes of their inclusion were not detailed in the provided sources [36].

A further exemplary protein structure that can form a CAR-CSD in the sense of the disclosure is provided by the intermediary domain (INT) and the death domain (DD) of MyD88 and the intracellular (cytoplasmic) domain of CD40. These domains represent an example of trans-costimulation provided by small molecule-mediated aggregation, offering a novel approach to CAR design. The specific impacts of these domains on CAR T-cell activity and fate were not detailed in the provided sources [36].

The co-stimulatory domain of a CAR protein in the sense of the disclosure is therefore usually composed of the cytoplasmic domain of a protein involved in immune cell activation and expansion. Many of these proteins in T-cells are either members of the immunoglobin super family or the TNF receptor family. Common motifs include YxxL/I, YMFM and proline rich domains that, through PI3K, AKT, ERK NFκB, or NFAT pathway signaling, can activate immune cell expansion and differentiation to more mature phenotypes.

CAR protein in the sense of the disclosure can include different combinations of domains in various configurations depending on the cells the CAR protein is configured to target.

For example, types and positioning of costimulatory domains within the CAR construct can significantly influence a CAR T-cell function. For example, the domain proximal to the cell membrane has a dominant effect on the cytokine profile [36]. The choice of costimulatory domains can also significantly impact the therapeutic efficacy and side effect profile of CAR immune cells therapies. For instance, CARs incorporating the 4-1BB domain tend to have a lower risk of inducing severe toxicities and promote longer-lasting T-cell responses. In contrast, CARs with CD28 domains can drive more rapid T-cell activation but may also lead to quicker T-cell exhaustion [28]. Therefore, the choice and combination of costimulatory domains, along with their positioning within the CAR construct, are crucial for optimizing the function, persistence, and efficacy of CAR T-cells. These domains include CD28, 4-1BB, ICOS, OX40, CD27, MyD88, and CD40, each contributing uniquely to the CAR T-cell's activation and antitumor response.

CAR proteins in the sense of the disclosure encompass chimeric construct of all generations. In particular, CAR proteins in the sense of the disclosure encompass first-generation CARs which contained an scFv CAR-ARD, only the CD3ζ signaling domain.

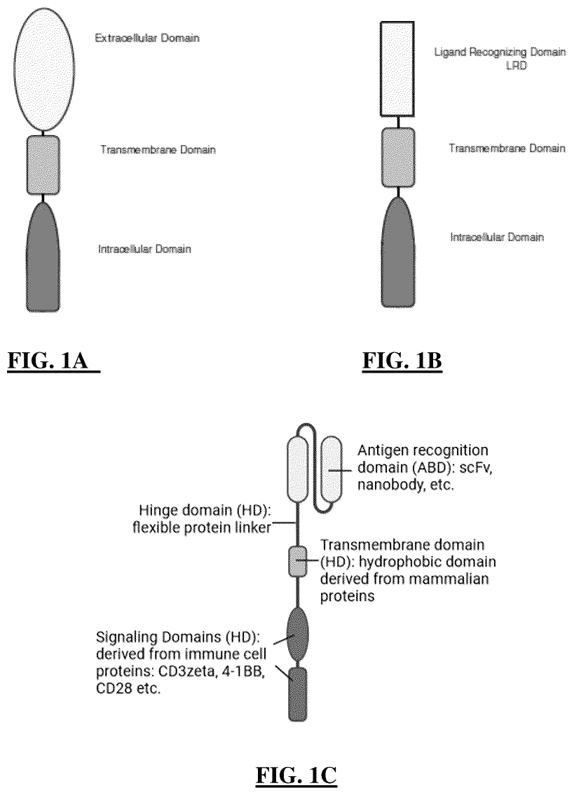

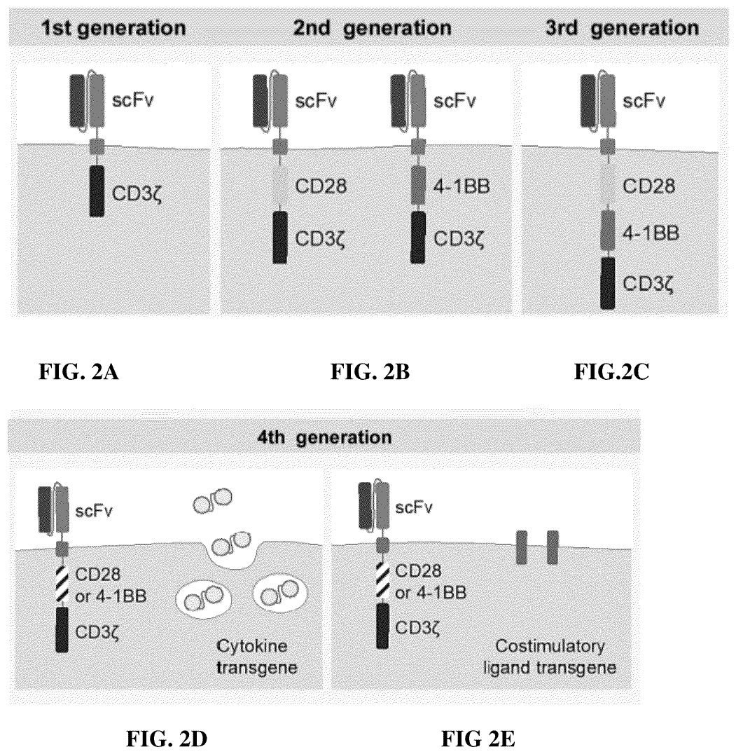

CAR proteins in the sense of the disclosure also encompass second- and third-generation CARs which incorporate one or more costimulatory domains in addition to CD35, to enhance T-cell proliferation, persistence, and overall efficacy. Common costimulatory domains include CD28, 4-1BB(CD137), and OX40 (CD134) [[28] (see Example 1 and FIG. 1C and Example 2, FIGS. 2A to 2E) which can be selected in combination to T-cell activation and function. For example, third-generation CARs can include CD33 along with two co-stimulatory domains such as CD28 and 4-1BB, aiming to enhance the overall therapeutic efficacy of the CAR T-cells. The CD28 and 4-1BB intracellular signaling domains are pivotal in defining the characteristics of CAR T-cells, such as their potency, persistence, and ability to resist exhaustion.

As another example, third generation CARs can include combinations and different positioning of CSD domains within the CAR construct including domains to alter physical distances between CSD domains to influence cytokine profiles and T-cell function [36] (see Example 1 and FIG. 1C and Example 2, FIGS. 2A to 2E) in particular based on the specific requirements of the therapy, including the type of cancer being targeted and the desired immune response as will be understood by a skilled person (see Example 1, FIG. 1C, and Example 2 FIGS. 2A to 2E).

As an additional example, the development of fourth-generation CARs (also known as T-cells engineered for antigen-directed activation and costimulation (TRUCKs)), and modifications to the base CAR structure to target multiple antigens or to modulate cytokine secretion, represent the cutting edge of CAR T-cell therapy research [26, 28]. These advancements are directed of making CAR T-cell therapies more effective against a broader range of cancers, including solid tumors, and reducing the risk of side effects such as cytokine release syndrome and neurotoxicity.

In some examples, CARs can comprise fusions of single chain variable fragments (scFv) derived monoclonal antibodies, fused to CD3ζ transmembrane and intracellular domain. Examples of CARs suitable to be comprised within a CAR-NLPs of the disclosure include CARs that bind to CD3, CD19, CD22, CD30, CD123, B cell maturation antigen (BCMA), GD2, mesothelin, EGVRVIII, HER2, e-MET, PD-L1, and other tumor associated antigens.

Exemplary of tumor associated antigens that can be targeted by CAR-NLPs of the disclosure can include mesothelin, EGFRVIII, TSHR, CD19, CD123, CD22, CD30, CD171, CS-1, CLL-1, CD33, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), MUCI, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEMI/CD248, TEM7R, CLDN6, GPRC5D, CXORF61, CD97, CD 179a, ALK, Polysialic acid, PLACI, GloboH, NY-BR-1, UPK2, HAVCR, ADRB3, PANX3, GPR20, LY6K, OR51E2, TARP, WTI, NY-ESO-1, LAGE-la, MAGE-A1, legumain, HPV E6,E7, MAGE A1, ETV6-AML, sperm protein 17, XAGEI, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB 1, BORIS, SART3, PAX5, OY-TESI, LCK, AKAP-4, SSX2, RAGE-1, human telomerase reverse transcriptase, RUI, RU2, intestinal carboxyl esterase, mut hsp70-2, CD79a, CD79b, CD72, LAIRI, FCAR, LILRA2, CD300LF, CLEC12A, BST2, EMR2, LY75, GPC3, FCRL5, and IGLLI. In these embodiments, the ABD of the CAR will be an scFv, nanobody, DARPin, ligand or other molecule capable of binding these tumor antigens.

In some embodiments, the CAR-NLPs can comprise CAR configured to bind to molecules expressed on the surface of tumor cells, including CD20, CD22, CD33, CD2, CD3, CD4, CD5, CD7, CD8, CD45, CD52, CD38, CS-1, TIM3, CD123, mesothelin, folate receptor, HER2-neu, epidermal-growth factor receptor, and epidermal growth factor receptor. In some embodiments, the immune activating receptor is a CAR (e.g., anti-CD19-4-1BB-CD3} CAR). In certain embodiments, the immune activating receptor comprises an antibody or antigen-binding fragment thereof (e.g., scFv) that binds to molecules expressed on the surface of tumor cells, comprising, CD20, CD22, CD33, CD2, CD3, CD4, CD5, CD7, CD8, CD45, CD52, CD38, CS-1, TIM3, CD123, mesothelin, folate receptor, HER2-neu, epidermal-growth factor receptor, and epidermal growth factor receptor. In these embodiments, the ABD of the CAR will be an scFv, nanobody, DARPin, ligand or other molecule capable of binding these cell surface tumor antigens.

In some embodiments, the CAR-NLPs can comprise CAR configured to bind to molecules expressed on the surface of immune cells, comprising CD19, CD20, CD22, CD72, and CD7 or other molecules found on the surface of B-cells, CD2, CD3, CD4, CD5, CD7, and/or CD8 or other molecules found on the surface of T-cells, CD9 or other markers of Eosinophils, CD63 and CD203 or other markers of mast-cells and basophils. In these embodiments, the ABD of the CAR will be an scFv, nanobody, DARPin, ligand or other molecule capable of binding these cell surface tumor antigens.

In some embodiments, the CAR-NLPs can comprise CAR configured to bind to molecules expressed on fibroblast or activated myofibroblasts, such as fibroblast associated protein. In these embodiments, the ABD of the CAR will be an scFv, nanobody, DARPin, ligand or other molecule capable of binding these fibroblast or myofibroblast cell surface antigens.

In some embodiments, the CAR-NLPs can comprise CAR configured to bind to molecules expressed on senescent-cells including urokinase plasminogen activator receptor. In these embodiments, the ARD of the CAR will be an scFv, nanobody, DARPin, ligand or other molecule capable of binding these cell surface tumor antigens.

In some embodiments, the CAR-NLPs can comprise CAR configured to bind to molecules expressed on the coat of pathogens that can be recognized by the immune system. In these embodiments, the ARD of the CAR will be an scFv, nanobody, DARPin, ligand or other molecule capable of binding these pathogen cell surface antigens. In some embodiments of these CARs, the ARD will be a scFv isolated from a neutralizing antibody against the pathogen.

The inventors have found that despite the relative ease with which CAR proteins are expressed in target-cells through genetic engineering techniques, CAR proteins are insoluble in aqueous medium. When cell-free synthesis of CAR protein was performed in isolation (without nanodisc components) the inventors were able to generate detectable CAR protein, but <1% of this protein is soluble in aqueous buffers.

The inventors have also surprisingly found that despite the known ability of nanolipoprotein particles to solubilize transmembrane protein structure, CAR proteins synthesized in the presence of nanodisc components do not generate soluble CAR-NLPs in aqueous buffers, without significant non-obvious process modification.

The inventors have further surprisingly found that CAR can be incorporated within nanolipoprotein particles configured for protein delivery of the CAR proteins in a functional form according to methods of the disclosure.

The inventors have additionally surprisingly found that procedures that can be used to increase solubility in CARs can also increase solubility for other immunoreceptors and transmembrane proteins comprising a transmembrane domain which increases their loading in NLPs obtained by existing methods to enable efficient delivery to immune cells.

As will be understood by a skilled person, the data and results obtained using the particularly insoluble and unmanageable single-pass ICTM CAR support a strong expectation that the disclosed strategies will improve both expression yield and solubility not only for other single-pass ICTMs, but also for multi-pass and other ICTM classes. The successful stabilization and recovery of such a challenging target underscores the broader applicability of the methods across diverse ICTM topologies.

More specifically, the methods, systems, particles, and compositions described herein in connection with CARs are expected to be similarly effective for other single-pass ICTMs that share structural features with chimeric antigen receptors. Similar to CARs, these proteins exhibit a tendency toward insolubility or aggregation when expressed in isolation, and therefore are expected to benefit from the co-translational scaffold integration provided by the NLP platform.

ICTM proteins which are single pass and expected to be effectively solubilized in functional form and delivered to immune cells comprise TCRs, B cell receptors (BCRs), Killer cell immunoglobulin-like receptors (KIRs) Cytokine receptors, Fc receptor RTKs, Immunoreceptor-type chimeras and decoys as well as additional single pass ICTMs identifiable by a skilled person upon reading of the present disclosure.]

The term “T cell receptors” or “TCRs” as used herein indicates heterodimeric antigen receptors expressed on T cells, composed of a and B chains (or γ and δ chains) each containing a single transmembrane domain and variable extracellular domains that recognize peptide-MHC complexes.

The term B cell receptors or “BCRs” as used herein indicates B cell receptors are antigen receptors expressed on immature B cells and are comprised of single pass transmembrane domains with an extracellular immunoglobulin like structure, comprised of a heavy chain and light chain. KIRs are receptors expressed on NK cells with a single pass helical transmembrane domain and intra and extracellular signaling domains. In vitro expression of full-length TCR or BCR or KIR chains is generally hindered by insolubility, aggregation, mispairing, or improper folding. The methods disclosed herein are expected to be effectively used to express individual TCR chains, engineered heterodimers, or stabilized single-chain TCR variants within NLPs for functional or structural analysis.

Killer cell immunoglobulin-like receptors (KIRs) are a family of transmembrane glycoproteins predominantly expressed on natural killer (NK) cells and a subset of T lymphocytes, where they function as key regulators of immune surveillance and cytotoxic activity. These receptors recognize specific motifs on major histocompatibility complex (MHC) class I molecules expressed by potential target cells, modulating NK cell activation through either inhibitory or activating signaling pathways. Inhibitory KIRs possess long cytoplasmic tails containing immunoreceptor tyrosine-based inhibitory motifs (ITIMs), which, upon engagement with self-MHC class I molecules, suppress NK cell-mediated cytotoxicity and contribute to self-tolerance. In contrast, activating KIRs contain short cytoplasmic tails and rely on association with adaptor molecules such as DAP12 to transmit activating signals via immunoreceptor tyrosine-based activation motifs (ITAMs), thereby promoting NK cell effector functions including cytolysis and cytokine secretion. The genes encoding KIRs are located within the leukocyte receptor complex (LRC) on chromosome 19q13.4 and exhibit extensive polymorphism and haplotypic diversity, which influence individual immune responses in the contexts of infection, cancer, transplantation, and reproduction.

The term “cytokine receptors” as used herein indicates single-pass ICTM proteins which are cytokine receptors such as the interleukin-2 receptor alpha chain (CD25), IL-7 receptor alpha (CD127), and common gamma chain (yc/CD132). These receptors often form part of multisubunit complexes and are poorly soluble in isolation. The extracellular domains may be modular and ligand-responsive, and the NLP scaffold enables their expression and display in a native-like configuration.

The term “Fc receptors:” or “Fcγ receptors” indicate single pass ICTM such as CD16A (FcγRIIIa) and CD64 (FcγRI) are single-pass transmembrane proteins involved in immune complex binding and antibody-mediated cytotoxicity. These proteins are characterized by extracellular Ig-like domains and often include intracellular ITAM motifs. NLP-scaffolded Fc receptors may be used for binding assays or engineering of synthetic effector systems.

The term “Receptor tyrosine kinases (RTKs)” as used herein indicates certain RTKs, such as HER2/ErbB2, CD117 (c-Kit), and PDGFR, which exhibit similar topology to CARs with a large extracellular domain, a single transmembrane domain, and a cytoplasmic tyrosine kinase domain. Although multi-domain and catalytically active, the primary solubility challenge resides in their transmembrane and juxtamembrane regions, which are addressed by the NLP scaffold.]

The term Immunoreceptor-type chimeras and decoys: Other proteins with CAR-like structure include engineered decoy receptors (e.g., PD-1-Fc chimeras), synthetic immune checkpoint blockers, and multi-domain constructs designed for competitive binding or signaling disruption. These constructs typically comprise a functional extracellular ligand-binding domain fused to a transmembrane domain and optional intracellular signaling modules.

In single pass ICTM, the transmembrane domain and adjacent regions contribute to the poor solubility and aggregation tendency observed in cell-free or recombinant expression systems. By providing a discoidal lipid bilayer scaffold via apolipoprotein-stabilized NLPs, the methods described herein support co-translational incorporation of such ICTM proteins into a membrane-like environment, promoting proper folding, accessibility of extracellular domains, and optional downstream functional analysis.

The term “nanolipoprotein particle,” “nanodisc,” “rHDL,” or “NLP” as used herein indicates a supramolecular complex formed by a membrane forming lipid arranged in a lipid bilayer stabilized by a scaffold protein. The membrane forming lipids and scaffold protein are components of the NLP. In particular, the membrane forming lipid component is part of a total lipid component (herein also membrane lipid component or lipid component) of the NLP together with additional lipids such as functionalized lipids and/or lysolipids, that can further be included in the NLPs as will be understood by a skilled person upon reading of the present disclosure. The scaffold protein component is part of a protein component of the NLP together with additional proteins such as membrane proteins, target proteins and other proteins that can be further included as components of the NLPs as will be understood by a skilled person upon reading of the present disclosure. Additional components can be provided as part of the NLP herein described as will be understood by a skilled person. In particular, the membrane lipid bilayer can attach membrane proteins or other amphipathic compounds through interaction of respective hydrophobic regions with the membrane lipid bilayer. The membrane lipid bilayer can also attach proteins or other molecules through anchor compounds or functionalized lipids as will be understood by a skilled person upon reading of the disclosure. In a nanolipoprotein particle, the membrane lipid bilayer can be confined in a discoidal configuration by the scaffold protein. Predominately discoidal in shape, nanolipoprotein particles typically have diameters between 5 to 60 nm and share uniform heights between 1 to 20 nm.

In particular, in embodiments herein described, the nanolipoprotein particle can be formed by a lipid bilayer confined in a discoidal configuration by a scaffold protein. In this configuration, the lipid bilayer confined by the scaffold protein can be 3-6 nanometers in thickness, the nanolipoprotein particle can have an overall diameter of 5-30 nanometers, and the scaffold protein on the particle can have a thickness of 1-5 nanometers. In some embodiments, an entire NLP structure can be up to 600 kilodaltons in molecular weight.

The particular membrane forming lipid, scaffold protein, the lipid to protein ratio, and the assembly parameters determine the size and homogeneity of nanolipoprotein particles as will be understood by a skilled person. In the nanolipoprotein particle the membrane forming lipid are typically arranged in a membrane lipid bilayer confined by the scaffold protein in a discoidal configuration as will be understood by a skilled person.

The term “membrane forming lipid” or “amphipathic lipid” as used herein indicates a lipid possessing both hydrophilic and hydrophobic moieties that in an aqueous environment assembles into a lipid bilayer structure that consists of two opposing layers of amphipathic molecules known as polar lipids. Each polar lipid has a hydrophilic moiety, i.e., a polar group such as, a derivatized phosphate or a saccharide group, and a hydrophobic moiety, i.e., a long hydrocarbon chain. Exemplary polar lipids include phospholipids, sphingolipids, glycolipids, ether lipids, sterols, alkylphosphocholines, and the like. Amphipathic lipids include, but are not limited to, membrane lipids, i.e., amphipathic lipids that are constituents of a biological membrane, such as phospholipids like dimyristoylphosphatidylcholine (DMPC) or dioleoylphosphoethanolamine (DOPE) or dioleoylphosphatidylcholine (DOPC), or dipalmitoylphosphatidylcholine (DPPC). In a preferred embodiment, the lipid is dimyristoylphosphatidylcholine (DMPC).

In some embodiments, the membrane forming lipids component of the lipid component lipids such as phospholipids, preferably including at least one phospholipid, typically soy phosphatidylcholine,, egg phosphatidylcholine, soy phosphatidylglycerol, egg phosphatidylglycerol, palmitoyl-oleoyl-phosphatidylcholine distearoylphosphatidylcholine, or distearoylphosphatidylglycerol. Other useful phospholipids include, e.g., phosphatidylcholine, phosphatidylglycerol, sphingomyelin, phosphatidylserine, phosphatidic acid, phosphatidylethanolamine, lysolecithin, lysophosphatidylethanolamine, phosphatidylinositol, cephalin, cardiolipin, cerebrosides, dicetylphosphate, diolcoylphosphatidylcholine, dipalmitoylphosphatidylcholine, dipalmitoylphosphatidylglycerol, dioleoylphosphatidylglycerol, stearoyl-palmitoyl-phosphatidylcholine, di-palmitoyl-phosphatidylethanolamine, distearoyl-phosphatidylethanolamine, di-myrstoyl-phosphatidylserine, di-myrstoyl-phosphatidylserine and diolcyl-phosphatidylcholine.

Additionally, exemplary membrane forming lipids that can be comprised in various combinations together with one or more lysolipids comprise 1,2-dipalmitoyl-sn-glycero-3-phosphocholine, 1,2-dimyristoyl-sn-glycero-3-phosphocholine, 1,2-dilauroyl-sn-glycero-3-phosphocholine, 1,2-didecanoyl-sn-glycero-3-phosphocholine, 1,2-dierucoyl-sn-glycero-3-phosphocholine, 1,2-dioleoyl-sn-glycero-3-phosphocholine, 1,2-dipalmitoleoyl-sn-glycero-3-phosphocholine, 1,2-dimyristoleoyl-sn-glycero-3-phosphocholine, 1-stearoyl-2-oleoyl-sn-glycero-3-phosphocholine, 1-stearoyl-2-myristoyl-sn-glycero-3-phosphocholine, 1-palmitoyl-2-oleoyl-sn-glycero-3-phosphocholine, egg phosphatidylcholine extracts, soy total lipid extracts, soy polar lipid extracts, soy phosphatidylcholine extracts, heart total lipid extracts, heart polar lipid extracts, heart phosphatidylcholine extracts, brain phosphatidylcholine extracts, liver phosphatidylcholine extracts, 1,2-distearoyl-sn-glycero-3-phosphate, 1,2-dipalmitoyl-sn-glycero-3-phosphate, 1,2-dimyristoyl-sn-glycero-3-phosphate, 1,2-dilauroyl-sn-glycero-3-phosphate, 1-palmitoyl-2-oleoyl-sn-glycero-3-phosphate, 1-stearoyl-2-oleoyl-sn-glycero-3-phosphate, 1,2-dipalmitoyl-sn-glycero-3-phosphocthanolamine, 1,2-dimyristoyl-sn-glycero-3-phosphoethanolamine, 1,2-dilauroyl-sn-glycero-3-phosphoethanolamine, 1,2-diolcoyl-sn-glycero-3-phosphoethanolamine, 1,2-dipalmitoleoyl-sn-glycero-3-phosphoethanolamine, Egg phosphatidylethanolamine extract, soy phosphatidylethanolamine extract, heart phosphatidylethanolamine extract, brain phosphatidylethanolamine extract, 1,2-distearoyl-sn-glycero-3-phospho-(l′-rac-glycerol), 1,2-diolcoyl-sn-glycero-3-phospho-(l′-rac-glycerol), 1,2-dipalmitoyl-sn-glycero-3-phospho-(l′-rac-glycerol), 1,2-dimyristoyl-sn-glycero-3-phospho-(1′-rac-glycerol), 1,2-dilauroyl-sn-glycero-3-phospho-(l′-rac-glycerol), 1-palmitoyl-2-olcoyl-sn-glycero-3-phospho-(l′-rac-glycerol), egg phosphatidylglycerol extract, soy phosphatidylglycerol extract, 1,2-distearoyl-sn-glycero-3-phospho-L-serine, 1,2-diolcoyl-sn-glycero-3-phospho-L-serine, 1,2-dipalmitoyl-sn-glycero-3-phospho-L-serine, 1,2-dimyristoyl-sn-glycero-3-phospho-L-serine, 1,2-dilauroyl-sn-glycero-3-phospho-L-serine, 1-palmitoyl-2-oleoyl-sn-glycero-3-phospho-L-serine, soy phosphatidylserine extract, brain phosphatidylserine extract, 2-((2,3-bis(oleoyloxy) propyl)dimethylammonio)ethyl hydrogen phosphate, cholesterol, ergosterol, sphingolipids, ceramides, sphingomyelin, gangliosides, glycosphingolipids, 1,2-dioleoyl-3-trimethylammonium-propane, 1,2-di-O-octadecenyl-3-trimethylammonium propane.

In some embodiments, non-phosphorus containing lipids can also be used as membrane forming lipids in the CAR-t-NLPs herein described, e.g., stearylamine, docecylamine, acetyl palmitate, and fatty acid amides. Additional membrane forming lipids suitable for use in providing NLPs are well known to persons of ordinary skill in the art and are cited in a variety of well-known sources, e.g., McCutcheon's Detergents and Emulsifiers and Mccutcheon's Functional Materials, Allured Publishing Co., Ridgewood, N.J., both of which are incorporated herein by reference.

In preferred embodiments, the immune NLPs comprise lipids which are known or expected to increase yield and/or functionality of the resulting immune NLPs as shown for example in Example 17, herein also indicated as preferred lipids. As used herein, a “preferred lipid” refers to a synthetic or semi-synthetic amphipathic molecule that possess a zwitterionic phosphatidylcholine headgroup; comprise saturated or monounsaturated acyl chains with 12-22 carbon atoms; and/or exhibit liquid-disordered phase behavior at the reaction temperature or at a temperature near but below the reaction temperature (e.g., 25-37° C.)(see Example 17).Among the preferred lipids, zwitterionic PC headgroup, saturated and/or monounsaturated, 12-22 carbon atoms lipid tails are most preferred as will be understood by a skilled person upon reading of the present disclosure.

The wording “zwitterionic phospholipid” refers to a lipid molecule having both a positively charged quaternary ammonium group and a negatively charged phosphate group under physiological pH, resulting in a net neutral charge. Preferred zwitterionic lipids comprise phosphatidylcholine (PC) headgroups and acyl chains selected to promote bilayer formation under NLP-relevant conditions. Exemplary members include: 1,2-dimyristoyl-sn-glycero-3-phosphocholine (DMPC) 1-palmitoyl-2-oleoyl-sn-glycero-3-phosphocholine (POPC), 1,2-dipalmitoyl-sn-glycero-3-phosphocholine (DPPC), 1,2-diolcoyl-sn-glycero-3-phosphocholine (DOPC).

The wording “saturated or monounsaturated acyl chain lipid” refers to a lipid molecule comprising one or more fatty acyl chains that contain either: (i) no carbon-carbon double bonds (fully saturated), or (ii) a single cis-configured carbon-carbon double bond (monounsaturated). Each acyl chain may contain from 12 to 22 carbon atoms, and may be chemically attached to a glycerol or other lipid backbone via an ester, ether, or amide linkage. Representative examples comprise Saturated lipids such as 1,2-dimyristoyl-sn-glycero-3-phosphocholine (DMPC, 14:0/14:0) and 1,2-dipalmitoyl-sn-glycero-3-phosphocholine (DPPC, 16:0/16:0), as well as Monounsaturated lipids: such as 1-palmitoyl-2-oleoyl-sn-glycero-3-phosphocholine (POPC, 16:0/18:1) 1,2-dioleoyl-sn-glycero-3-phosphocholine (DOPC, 18:1/18:1)

In some embodiments herein described, lipids comprising saturated or monounsaturated fatty acid chains are preferred for promoting membrane fluidity and lateral diffusion within the NLP bilayer while avoiding excessive rigidity or phase separation. Saturated chains may comprise C12 to C18 fatty acyl groups, while monounsaturated chains contain a single cis double bond. Exemplary lipids include: DMPC (14:0/14:0), DPPC (16:0/16:0), POPC (16:0/18:1), DOPC (18:1/18:1) Saturated or monounsaturated lipids promote fluid-phase membrane properties at reaction temperatures (typically 30-37° C.) conducive to protein insertion and NLP self-assembly.

The wording “Liquid-disordered (Ld) phase lipids” are defined as lipids which, at experimental temperatures used for cell-free protein synthesis (e.g., 25-37° C.), form bilayers in a liquid-disordered phase, characterized by lateral mobility and lack of ordered lipid packing. Ld-phase behavior supports spontaneous NLP formation and membrane protein insertion. Exemplary Ld-phase lipids include: POPC DOPC DMPC, under conditions exceeding its transition temperature (˜23° C.). Lipids that form gel-phase (LB) bilayers under the same conditions (e.g., DPPC at ≤37° C.) are preferably excluded or included only as minor components (<30 mol %) unless counterbalanced by more fluidizing lipids.

In particular, in preferred embodiments the preferred lipids are comprised in at least 60-90 mol % of the total lipid composition. The remaining 10⁻⁴⁰ mol % can include structurally compatible helper lipids, including minor fractions of anionic lipids or phase-modifying components as will be understood by a skilled person upon reading of the present disclosure.

In preferred embodiments herein described, preferred lipids are typically included in various combinations at a molar fraction of at least 40%, more preferably at least 50%, and most preferably between 60% and 90% of the total lipid composition used for immuno-NLP formation.

Zwitterionic lipids are included at a molar fraction of at least 40%, preferably at least 50%, and more preferably between 60% and 90%, in lipid mixtures formulated for immuno-NLP production. More Preferred formulations comprise >70 mol % neutral (zwitterionic) lipids in total.

In some preferred embodiments, the lipid composition of the NLP comprise low-charge or neutral lipid formulations which are formulations exhibiting low net negative charge are preferred to reduce non-specific aggregation of proteins or scaffold elements during NLP assembly. Accordingly in preferred embodiments, anionic lipids (e.g., phosphatidylglycerol derivatives) accordingly be included at less than 20-30 mol %, and preferably less than 10 mol %, to avoid electrostatic destabilization. Exemplary anionic lipids that can be optionally included in minor amounts include: 1,2-dimyristoyl-sn-glycero-3-phospho-(l′-rac-glycerol)(DMPG) and 1-palmitoyl-2-olcoyl-sn-glycero-3-phosphoglycerol (POPG).

In some preferred embodiments, lipid formulations are substantially free of sterols, sphingolipids, or non-phospholipid components, which are expected to interfere with scaffold binding, NLP curvature, or reproducibility. The term “substantially free” means containing less than 5 mol % total of such components. Excluded lipids include for example cholesterol sphingomyelin brain lipid extracts as will be understood by a skilled person upon reading of the present disclosure.

Such formulations provide reproducible bilayer composition for the incorporation of recombinant ICTMs in in vitro systems and are known or expected to contributes to the formation of nanolipoprotein particles (NLPs) suitable for the insertion, stabilization, and display of one or more immune cell transmembrane proteins (ICTMs), including but not limited to chimeric antigen receptors (CARs), T cell receptors, Fc receptors, or checkpoint proteins.

In some embodiments nanolipoprotein particles (NLPs) suitable for incorporation of membrane-associated or aggregation-prone polypeptides under cell-free expression conditions, the lipid component is formulated to comprise at least one bilayer-forming zwitterionic phosphatidylcholine. The lipid can be provided as small unilamellar vesicles and added to the reaction mixture at a final concentration of between about 1 mg/mL and about 5 mg/mL, such as 2 mg/mL

In some embodiments, preferred zwitterionic phosphatidylcholines are, for example, 1,2-dimyristoyl-sn-glycero-3-phosphocholine (DMPC), egg-derived phosphatidylcholine (EggPC), diolcoyl-phosphatidylcholine (DOPC), palmitoyl-olcoyl-phosphatidylcholine (POPC), and stearoyl-oleoyl-phosphatidylcholine (SOPC). In some of those embodiments The zwitterionic phosphatidylcholine should constitute at least about 50 mol % of the total lipid composition in order to support formation of discoidal NLPs under the assembly conditions described herein. In some of those embodiments, the lipid composition can further include up to about 20 mol % of one or more additional lipid components, including anionic phospholipids such as 1,2-dimyristoyl-sn-glycero-3-phospho-(l′-rac-glycerol)(DMPG) or 1,2-dioleoyl-sn-glycero-3-phospho-(l′-rac-glycerol)(DOPG), sterols such as cholesterol, or other helper lipids. The selection and proportion of these additional components should be such that bilayer integrity is retained and NLP formation is maintained in the presence of apolipoprotein and expressed protein components.

In some preferred embodiments, lipid components that disrupt bilayer formation, such as non-bilayer-forming lipids (e.g., dioleoyl-phosphatidylethanolamine (DOPE)) or micelle-forming lipids (e.g., lysolipids), are not used as primary constituents. These lipids can be included in minor amounts, for example less than about 5 mol %, where necessary to modulate curvature or fusogenicity, but are not relied upon for NLP scaffold formation.

In some embodiments, the lipid composition consists essentially of DMPC or EggPC alone, or in combination with 5-20 mol % of DMPG, to support optimal soluble expression and incorporation of chimeric antigen receptors into NLPs. Under these conditions, co-expression with apolipoprotein and incubation at temperatures between about 15° C. and about 25° C. results in the formation of discoidal NLPs containing folded, functional protein.

In nanodisc of the disclosure, the membrane forming lipids are assembled in a discoidal membrane lipid bilayer stabilized by a scaffold protein.

The term “scaffold protein” as used herein indicates any amphipathic protein that is capable of self-assembly with amphipathic lipids in an aqueous environment, organizing the amphipathic lipids into a bilayer disc, and comprise apolipoproteins, lipophorins, derivatives thereof (such as truncated and tandemly arrayed sequences) and fragments thereof (e.g. peptide fragments and synthetic peptides) which maintains the amphipathic nature and capability of self-assembly, such as apolipoprotein E4 (22Kd fragment), lipophorin III, apolipoprotein A-1 and the like. In general, scaffold proteins have an alpha helical secondary structure in which a plurality of hydrophobic amino acids form a hydrophobic face and a plurality of hydrophilic amino acids form an opposing hydrophilic face. In some embodiments, rationally designed amphipathic peptides and synthetic apolipoproteins which maintain an amphipathic structure and capability of self-assembly can serve as a scaffold protein of the NLP.

The term “apolipoprotein” as used herein indicates an amphipathic protein that binds lipids to form lipoproteins. The term “amphipathic” pertains to a molecule containing both hydrophilic and hydrophobic properties. Exemplary amphipathic molecules comprise molecules having hydrophobic and hydrophilic regions/portions in its structure. Examples of biomolecules that are amphipathic include, but are not limited to, phospholipids, cholesterol, glycolipids, fatty acids, bile acids, saponins, and additional lipids identifiable by a skilled person.

A “lipoprotein” as used herein indicates a biomolecule assembly that contains both proteins and lipids. In particular, in lipoproteins, the protein component surrounds or solubilizes the lipid molecules enabling particle formation. Exemplary lipoproteins include the plasma lipoprotein particles classified under high-density (HDL) and low-density (LDL) lipoproteins, which enable fats and cholesterol to be carried in the blood stream, the transmembrane proteins of the mitochondrion and the chloroplast, and bacterial lipoproteins. In particular, the lipid components of lipoproteins are insoluble in water, but because of their amphipathic properties, apolipoproteins such as certain Apolipoproteins A and Apolipoproteins B and other amphipathic protein molecules can organize the lipids in a bilayer orientation with exposed hydrophilic moieties, creating the lipoprotein particle that is itself water-soluble, and can thus be carried through water-based circulation (e.g. blood, lymph in vivo or in vitro).

Apolipoproteins known to provide the protein components of the lipoproteins can be divided into six classes and several sub-classes, based on the different structures and functions. Exemplary apolipoprotein known to be able to form lipoproteins comprise Apolipoproteins A (apo A-I, apo A-II, apo A-IV, and apo A-V), Apolipoproteins B(apo B48 and apo B100), Apolipoproteins C (apo C-I, apo C-II, apo C-III, and apo C-IV), Apolipoproteins D, Apolipoproteins E, and Apolipoproteins H. For example, apolipoproteins B can form low-density lipoprotein particles, and have mostly beta-sheet structure and can associate with lipid droplets irreversibly, while Apolipoprotein Al comprise alpha helices and can associate with lipid droplets reversibly forming high-density lipoprotein particles.

The term “protein” as used herein indicates a polypeptide with a particular secondary and tertiary structure that can interact with another molecule and in particular, with other biomolecules including other proteins, DNA, RNA, lipids, carbohydrates, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. In particular, in a protein, the polypeptide provides the primary structure of the protein, wherein the term “primary structure” of a protein refers to the sequence of amino acids in the polypeptide chain covalently linked to form the polypeptide polymer. A protein “sequence” indicates the order of the amino acids that form the primary structure. Covalent bonds between amino acids within the primary structure can include peptide bonds or disulfide bonds, and additional bonds identifiable by a skilled person. Polypeptides in the sense of the present disclosure are usually composed of a linear chain of alpha-amino acid residues covalently linked by peptide bond or a synthetic covalent linkage. The two ends of the linear polypeptide chain encompassing the terminal residues and the adjacent segment are referred to as the carboxyl terminus (C-terminus) and the amino terminus (N-terminus) based on the nature of the free group on each extremity. Unless otherwise indicated, counting of residues in a polypeptide is performed from the N-terminal end (NH₂-group), which is the end where the amino group is not involved in a peptide bond to the C-terminal end (—COOH group) which is the end where a COOH group is not involved in a peptide bond. Proteins and polypeptides can be identified by x-ray crystallography, direct sequencing, immunoprecipitation, and a variety of other methods as understood by a person skilled in the art. Proteins can be provided in vitro or in vivo by several methods identifiable by a skilled person. In some instances where the proteins are synthetic proteins in at least a portion of the polymer two or more amino acid monomers and/or analogs thereof are joined through chemically-mediated condensation of an organic acid (—COOH) and an amine (—NH₂) to form an amide bond or a “peptide” bond.

As used herein the term “amino acid”, “amino acid monomer”, or “amino acid residue” refers to organic compounds composed of amine and carboxylic acid functional groups, along with a side-chain specific to each amino acid. In particular, alpha- or α-amino acid refers to organic compounds composed of amine (—NH₂) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D and L optical isomers.

In some embodiments, the scaffold proteins can contain amino acid additions, deletions, or substitutions. In other embodiments, the scaffold proteins can be derived from various species and more particularly derived from human, mouse, rat, guinea pig, rabbit, cow, horse, pig, dog, koala, and non-human primates.

In some embodiments, membrane forming lipids can be comprised within a CAR-NLP stabilized by scaffold proteins such as human derived apoE4, truncated versions of human derived apoE4 (e.g. apoE422k), human derived apoE3, truncated versions of human derived apoE3 (e.g. apoE322k), human derived apoE2, truncated versions of human derived apoE2 (e.g. apoE222k), human derived apoAl, truncated versions of human derived apoAl (e.g. Δ49ApoA1, MSP1, MSPIT2, MSPIE3D1), mouse derived apoE4, truncated versions of mouse derived apoE4 (e.g. apoE422k), mouse derived apoE3, truncated versions of mouse derived apoE3 (e.g. apoE322k), mouse derived apoE2, truncated versions of mouse derived apoE2 (e.g. apoE222k), mouse derived apoAl, truncated versions of mouse derived apoAl (e.g. Δ49ApoA1, MSP1, MSPIT2, MSP1E3D1), rat derived apoE4, truncated versions of rat derived apoE4 (e.g. apoE422k), rat derived apoE3, truncated versions of rat derived apoE3 (e.g. apoE322k), rat derived apoE2, truncated versions of rat derived apoE2 (e.g. apoE222k), rat derived apoA1, truncated versions of rat derived apoAl (e.g. Δ49ApoA1, MSP1, MSPIT2, MSPIE3D1), lipophorins (e.g. B. mori, M. sexta), synthetic cyclic peptides that mimic the function of apolipoproteins. Other apolipoproteins, as will be understood for a skilled person, can be used to form NLP, including but not limited to apoB and apoC.

Methods to assemble NLPs are known and identifiable by a skilled person. For example, methods to provide nanolipoprotein particles known to a skilled person to provide one or more NLPs presenting one or more membrane proteins, comprise the methods described in U.S. Patent Publication No. 2009/0192299 related to methods and systems for assembling, solubilizing and/or purifying a membrane associated protein in a nanolipoprotein particle, which comprise a temperature transition cycle performed in presence of a detergent, wherein during the temperature transition cycle the nanolipoprotein components are brought to a temperature above and below the gel to liquid crystallization transition temperature of the membrane forming lipid of the nanolipoprotein particle. In some embodiments, verification of inclusion of a membrane protein can be performed using the methods and systems for monitoring production of a target protein in a nanolipoprotein particle described in U.S. Patent Publication No. 2009/0136937, filed on May 9, 2008, as Ser. No. 12/118,530, which is incorporated by reference in its entirety.

In some exemplary methods described in U.S. Patent Publication No. 2009/0192299 and U.S. Patent Publication No. 2009/0136937, filed on May 9, 2008 as Ser. No. 12/118,530, assembly of a nanolipoprotein particle can be performed to comprises an active target molecule, such as an immunogen, a drug, a contrast agent or another molecule of interest, comprised as a membrane protein or as an active target molecule attached to functionalized amphipathic compounds in the membrane lipid bilayer, in a configuration resulting having the active target molecule presented on the nanolipoprotein particle. In particular, the active target molecule can be a target protein having a hydrophobic region and be presented on the nanolipoprotein particle attached to the membrane lipid bilayer through interaction of the target protein hydrophobic region with the membrane lipid bilayer. In addition, or in the alternative, the active target molecule can be an active target molecule presented on the nanolipoprotein particle attached to one or more functionalized membrane forming lipid through anchor compounds as described in U.S. Pat. No. 8,883,729, issued on Nov. 11, 2014, and in U.S. Pat. No. 8,889,623, issued on Nov. 18, 2014, each of which is incorporated by reference in its entirety.

The inventors however have surprisingly found that using previous cell-free methods for synthesizing membrane proteins in nanodiscs did not generate soluble CARs embedded in nanodiscs.

The cell-free synthesis methods described herein [9, 40] generate CAR-NLPs with a molar ratio of ratio of <1CAR: 10NLPs and a solubility of <20% (Scc Example 5, and FIG. 8). CAR synthesis using the cell-free synthesis methods described herein [9, 40] in the presence of preformed nanodiscs did not improve CAR yield, CAR: NLP ratio or solubility. Generation of CAR-NLPs using previously published methods [41] and removal of insoluble fraction by centrifugation was not sufficient for generating a soluble suspension of CAR NLP with a 1:10 CAR: NLP molar ratio and a minimum concentration of 10 nanomolar as >80% CAR protein is lost using centrifugation.

The inventors have however surprisingly found that the correct assembly of CAR proteins in an NLP, in a correct conformation (capable of binding to a ligand specified by the receptor) with (a) the molar ratio of CAR to nanodisc of ≥20:100 (b) an initial CAR concentration of at least 10 nanomolar (c) a solubility of >90% can be achieved by operating the following result affective variables: (a) the composition of the lipid component of the NLP, (b) the temperature of incubation, and (c) the timing of the incubation for cell free synthesis, (d) inclusion of telodendromers.

In embodiments of methods and systems of manufacturing the CAR-NLPs of the disclosure, the assembly factors are thus result effective variables for the correct and successful inclusion of CAR in NLPs. In particular, the assembly are interrelated as they create in combination one with the other, a lipid environment during NLPs assembly that enables and drives the correct assembly of the CAR proteins in the lipid bilayer of the NLPs.

For example, in preferred embodiments assembly of CAR protein in an Immuno-NLP where CAR includes a functional ARD with an scFv, require reducing assembly conditions since the scFv folding requires disulfide bonding for ligand recognition [42], in contrast with standard assembly procedures [9, 40, 41, 43]. The inventors have operated on the assumption that scFv aggregation problems arise from these disulfide bonds. Thus, in preferred embodiments, lower synthesis temperature promotes CAR-lipid association and reduces scFv aggregation.

Additionally, the method identified for the correct assembly of CAR proteins has been surprisingly found to allow loading of CARs and other transmembrane proteins in Immuno-NLPs at a concentration of at least 10 nanomolar and possibly at least 17.5 nanomolar concentration which appears to be required to allow effective transfer of a functional immune cell transmembrane protein and more surprisingly a CAR protein into an immune cell.

Accordingly, Immuno-NLPs of the disclosure and in particular CAR-NLPs of the disclosure can be produced by a cell-free method/system in which polynucleotide encoding for immune cell transmembrane protein and the scaffold protein are expressed in the presence of lipids and other NLPs components for a time and under conditions that allow assembly of the NLP.

As used herein, the wording “cell-free expression”, “cell-free translation”, “in vitro translation” or “IVT” refer to at least one compound or reagent that, when combined with a polynucleotide encoding a polypeptide of interest, allows in vitro translation of said polypeptide/protein of interest.

The term “polynucleotide” as used herein indicates an organic polymer composed of two or more monomers including nucleotides, or analogs thereof. The term “nucleotide” refers to any of several compounds that consist of a ribose (ribonucleotide) or deoxyribose (deoxyribonucleotides) sugar joined to a purine or pyrimidine base and to a phosphate group, and that are the basic structural units of nucleic acids. The term “nucleotide analog” refers to a nucleotide in which one or more individual atoms have been replaced with a different atom with a different functional group. Accordingly, the term polynucleotide includes nucleic acids of any length of DNA or RNA analogs and fragments thereof. A polynucleotide of three or more nucleotides is also called nucleotidic oligomers or oligonucleotide.

In particular, co-expression of both scaffold protein and immuno-NLPs of the disclosure and in particular CAR in presence of phospholipids with or without surfactant/detergent can be performed in a ‘one-pot’ reaction that generates, in situ, both scaffold protein and target membrane protein. NLP self-assembly will ensue using phospholipids already in the reaction mixture.

Following the related translation the protein components and the membrane forming lipid of the Immuno-NLP such as a CAR-NLP are generally able to self-assemble in a biological (largely aqueous) environment according to the thermodynamics associated with water exclusion (increasing entropy) during hydrophobic association. In the methods and systems herein provided, the amphipathic lipid and the protein components of the NLP are allowed to assembly in a cell-free expression system.

In some embodiments, the polynucleotides encoding the Immuno-NLPs of the disclosure and in particular CAR and/or the scaffold protein or other proteins can comprise an engineered polynucleotide designed such that the resulting protein can be expressed as a full-length protein. In some embodiments, the polynucleotide is an engineered polynucleotide designed to encode a CAR expressed as a fusion, or chimeric protein product (i.e., it is joined via a peptide bond to a heterologous protein sequence of a different protein), for example to facilitate purification or detection. A chimeric product can be made by ligating the appropriate nucleic acid sequences encoding the desired amino acid sequences to each other using standard methods and expressing the chimeric product. In particular, in some embodiments, the polynucleotide can be engineered so that the CAR or other immune transmembrane protein is labeled or tagged. Labeling or tagging can be performed with methods that include, for example, FRET pairs, NHS-labeling, fluorescent dyes, and biotin as well as coding for a poly-histidine-tag to enable protein isolation and purification via established Ni-affinity chromatography.

In some embodiments herein described, the polynucleotide is a DNA molecule that can be in a linear or circular form and encodes the desired polypeptide under the control of a promoter specific to an enzyme such as an RNA polymerase, that is capable of transcribing the encoded portion of the DNA.

In embodiments where the polynucleotide is DNA, the DNA can be transcribed as part of the cell-free reactions or system. In those embodiments, the DNA contains appropriate regulatory elements, including but not limited to ribosome binding site, T7 promoter, and T7 terminator, and the reagents or compounds include appropriate elements for both transcription and translation reactions. In other embodiments where the polynucleotide is RNA, the RNA can be prepared prior to addition to the cell-free reactions/system, wherein the polypeptide of interest is produced, and the reagents or compounds include appropriate elements for translation reactions only.

Accordingly, as used herein, the term “cell-free expression”, “cell-free translation”, “in vitro translation” or “IVT” refer to methods and systems wherein the transcription and translation reactions are carried out independently, and to systems in which the transcription and translation reactions are carried out simultaneously in a non-cellular compartment, e.g., a glass vial.

In each of these methods and systems, the reagents or compounds typically include a cell extract capable of supporting in vitro transcription and/or translation as appropriate. In any case, the cell extracts contain all the enzymes and factors to carry out the intended reactions, and in addition, be supplemented with amino acids, an energy regenerating component (e.g. ATP), and cofactors, including factors and additives that support the solubilization of the protein of interest.

These systems are known in the art and can be identified by the skilled person upon reading of the present disclosure and exist for both eukaryotic and prokaryotic applications. Exemplary cell-free expression systems that can be used in connection with the methods and systems of the present disclosure includes but are not limited to commercial kits for various species such as extracts available from Invitrogen, Ambion, Qiagen and Roche Molecular Diagnostics, cellular extracts made from E. coli or wheat germ or rabbit reticulocytes, or prepared following protocols, such as published laboratory protocols, identifiable by a skilled person upon reading of the present disclosure.

In some embodiments, the cell-free system can operate in batch mode or in a continuous mode. In the batch mode, the reaction products remain in the system and the starting materials are not continuously introduced. Therefore, in batch mode, the system produces a limited quantity of protein. In a continuous mode instead, the reaction products are continuously removed from the system, and the starting materials are continuously restored to improve the yield of the protein products and therefore the system produces a significantly greater amount of product.

In some embodiments, CAR-NLPs herein described can be assembled by a translation method, where self-assembly of the NLPs can be achieved while the apolipoprotein or other scaffold protein is provided as a protein in a mixture also comprising one or more membrane forming lipids, one or more telodendrimers, a polynucleotide coding for the CAR, and a scaffold protein.

In some embodiments, CAR-NLPs herein described can be assembled by a translation method, where self-assembly of the NLPs can be achieved while the apolipoprotein or other scaffold protein is being translated from mRNA [44-46]. In this process, expression system lysates are mixed with the lipid and telodendrimer component of the NLP and plasmid DNA encoding the scaffold protein. The reaction can then be allowed to proceed until assembly occurs during apolipoprotein expression (e.g. for approximately 4 to 24 hours). The apolipoprotein typically contains an affinity tag (e.g. His-tag) for subsequent purification of the self-assembled NLP from the lysate.

The wording “cell-free lysate” when used in connection with preparation of NLPs indicates refers to a biologically active mixture derived from disrupted cells that retains the essential molecular machinery necessary for in vitro protein synthesis. These lysates typically contain ribosomes, tRNAs, aminoacyl-tRNA synthetases, translation factors, and other components required for efficient transcription and translation, while omitting intact cellular membranes and genomic DNA. When supplemented with appropriate cofactors, energy substrates, and template DNA or mRNA encoding target proteins such as apolipoproteins, the cell-free lysate enables the rapid and controlled synthesis of membrane-interacting or membrane-associated proteins in a defined reaction environment. For NLP preparation, cell-free expression systems facilitate the co-translational assembly of apolipoproteins with lipids, resulting in the spontaneous formation of discoidal nanolipoprotein complexes that mimic the structural and functional characteristics of high-density lipoproteins (HDLs). These systems are particularly advantageous for producing NLPs because they support the incorporation of amphipathic proteins without the need for detergents or membrane solubilization steps, allow precise control over reaction conditions, and are amenable to high-throughput or scalable formats. Cell-free lysates may be derived from a variety of sources, including Escherichia coli, wheat germ, or mammalian cells, depending on the desired post-translational modifications and yield.

All cell-free lysates that are compatible with the addition of lipids are expected to be suitable for the production of immune-NLPs, as contemplated in the present disclosure.

A “cell-free lysate being compatible with the addition of lipids” refers to a lysate preparation that permits the stable and functional incorporation of exogenous lipid components into the cell-free reaction mixture without impairing transcription, translation, or product assembly. Such compatibility requires that the lysate tolerate the physical and chemical properties of the lipid components-including concentration, composition, and formulation (e.g., liposomes, micelles, or detergent-solubilized lipids)-without causing aggregation, precipitation, or inhibition of protein synthesis. In the context of nanolipoprotein particle (NLP) production, a compatible cell-free lysate supports the co-translational or post-translational assembly of amphipathic or membrane-interacting proteins with added lipids, enabling the spontaneous formation of soluble NLPs with appropriate structural and functional properties. This compatibility may also reflect the lysate's ability to maintain reducing conditions, ion balance, and enzymatic activity in the presence of lipids, and may further include the absence of lipid-degrading enzymes or interfering hydrophobic aggregates.

Properties of the cell-free lysate that are expected to impact the solubility and yield of the immune-NLPs include, but are not limited to, the identity of the lysate host organism, the capacity to support post-translational modifications, the method of lysate preparation, and the presence or absence of endotoxins. In the present disclosure, two different lysates have been demonstrated to support the production of immune-NLPs having a CAR: NLP molar ratio greater than 0.2 and a soluble fraction greater than 0.2, indicating that multiple lysate systems are compatible with the generation of immuno-NLPs as described herein (see Example 26).

In some embodiments, the additives used in the cell-free reaction systems include any substance that improves the solubilization of the protein of interest and/or of any other protein components that are present in the reaction mixtures, any substance that may augment protein production and any substance that improves protein functions. Those additives include but are not limited to cofactors (e.g., retinal, heme), other proteins that facilitate modification (e.g., glycosylases, phosphatases, chaperonins) lipids, redox factors, detergents and protease inhibitors, and in particular, phospholipids such as dimyristoylphosphatidyl choline (DMPC) and the like, and surfactants/detergents such as cholate, triton X-100 and the like. Exemplary detergents that can be used for protein solubilization in the methods and systems herein disclosed include Heptanoyl-N-methyl-glucamide, Octanoyl-N-methyl-glucamide, Nonanoyl-Nmethyl-glucamide, n-Nonyl-b-D-gluco-pyranoside, N-Octyl-b-D-glucopyranoside, Octyl-b-D-thiogluco-pyranoside, NN-Dimethyldodecylamine-N-oxide and Glycerol. Additional additives that might be included in the reaction mixtures include labels and labeling molecule that can be used to label or tag the target protein and thus to enable the detection of the target protein through detection of a related labeling signal.

In some embodiments herein described, the CAR-NLPs of the disclosure can be provided by a method for producing a CAR-nanolipoprotein particle (CAR-NLP), comprising providing one or more membrane forming lipids within a lipid mixture, a polynucleotide coding for the CAR and a polynucleotide coding for a scaffold protein, mixing one or more membrane forming lipids with the polynucleotide coding for the immune cell transmembrane protein (ICTM polynucleotide) which in some preferred embodiments can comprise CAR protein (CAR polynucleotide), and the polynucleotide coding for the scaffold protein (SCP polynucleotide) with an in vitro cell-free translation system to provide a single reaction mixture, in which the ICTM polynucleotides and the SCP scaffold protein are incorporated to the cell free mix at between 0.1 μg/ml and 100 μg/ml with preferred ratios of 10⁻³⁰ ug/ml; and in a molar ratio (CAR: scaffold) of between 20:1 and 60:1; expandable to 2:1 to 600:1, in which the one or more membrane forming lipids at a total lipid concentration of from 0.1 mg/ml to 100 mg/ml, with 10 mg/ml-30 mg/ml preferred, and in which telodendrimers may be included, where the telodendrimer to cell free reaction mix is between 0.01 mg/ml and 1 g/ml with preferred ratios of 10 mg/ml to 40 mg/ml and translating the ICTM polynucleotide and the SCP polynucleotide within the single reaction mixture via the in vitro cell-free translation system at a temperature from 4° C. to 27° C., with 20° C. to 25° C. preferred, for a time from 2 hours to 168 hours with 12 to 24 hours preferred, with longer times for lower temperatures, wherein the mixing and translating are performed to allow self-assembly of the scaffold protein, the one or more membrane forming lipids into a nanolipoprotein particle, the nanolipoprotein particle comprising the CAR or within a discoidal membrane lipid bilayer formed by the one or more membrane forming lipids and stabilized by the scaffold protein, the membrane lipid bilayer attaching the CAR through interaction of a hydrophobic region of the CAR with the membrane lipid bilayer, as indicated by ability to purify CAR by purifying NLP.

In some embodiments, the ICTM polynucleotide and the ACP polynucleotide can be comprised in gene expression cassettes of a same or different engineered polynucleotide construct.

The term “gene cassette” refers to a mobile genetic element that includes at least one gene and a recombination site. Thus, a gene cassette may contain a single gene or multiple genes, potentially organized in an operon structure. A gene cassette can be transferred from one DNA sequence (usually on a vector) to another by ‘cutting’ the fragment out using restriction enzymes, transposase, CRISPR, viral and/or recombinase enzymes, and other nucleases, and then ‘pasting’ it into a new context using various molecular biology and cloning techniques (e.g., PCR, CRISPR, TALENs, ZFN). Gene cassettes can move within an organism's genome or be transferred to another organism in the environment through horizontal gene transfer.

A “gene expression cassette” is a type of gene cassette that includes regulatory sequences to be expressed by a transfected cell. After transformation, the expression cassette directs the cell's machinery to produce RNA and proteins. Some expression cassettes are designed for modular cloning of protein-encoding sequences, allowing the same cassette to be easily modified to produce different proteins. An expression cassette consists of one or more genes and the sequences that control their expression. Typically, an expression cassette includes at least three components: a promoter sequence, an open reading frame, and a 3′ untranslated region, which in eukaryotes usually contains a polyadenylation site. An expression cassette can be formed by a manipulable DNA fragment capable of expressing one or more genes of interest, optionally located between one or more sets of restriction sites. Gene expression cassettes, as used herein, typically include additional regulatory sequences beyond the promoter to regulate the expression of the gene or genes within the open reading frame, also referred to as the coding region of the cassette.

In polynucleotide constructs comprising ICTM polynucleotides and/or SCP polynucleotides, one of skill will be able to fine-tune the expression stoichiometry by adjusting the regulatory sequences and the number of cassettes within a construct, ensuring that the desired levels of gene expression are achieved as will be understood by a skilled person. Reduced synthesis temperature may warrant a different polynucleotide stoichiometry as will be understood by a skilled person.

Polynucleotide constructs in the sense of the disclosure are comprised in vectors such as plasmids, viral vectors, cosmids, Bacterial Artificial Chromosomes (BACs) and additional vectors identifiable by a skilled person.

In some embodiments, the lipid composition in the mixture can further be selected to improve solubility of CAR proteins or other immune cell transmembrane proteins.

In some embodiments, the ratio of CAR plasmid to Apolipoprotein plasmid can be selected to improve solubility of the CAR protein. For example, increasing CAR: Apo plasmid ratio from 20:1 to 60:1 can result in slightly higher solubility, though at the cost of reduction in overall yield.

In some embodiments, the scaffold protein is selected to define the size of empty NLPs. In particular, the scaffold protein and/or the membrane forming lipid can be selected so that the scaffold protein and the membrane forming lipid are contacted at a mass ratio of scaffold protein to membrane forming lipid from about 1:10 to about 1:1 to provide a particle having a size from 10 to 60 nm. In some embodiments, Lipophorin III lipoproteins may assemble into larger NLPs with diameters 10⁻³⁰ nm range, apolipoprotein Al NLPs range in size from 10⁻²⁵ nm, truncated A (1-49) Apolipoprotein A1 15-35 nm. Adjustment of protein to lipid ratios by increasing lipid will also increase the size of the NLP. An exemplary, procedure is illustrated in the examples section. Inclusion of CAR protein can cause up to 4-fold increase in size to the dimensions of an empty NLP. In some embodiments, the method further comprises providing one or more telodendrimer within the lipid mixture to form telo-nanolipoprotein particles (t-NLPs or t-NLPs). When telodendromers are included, the ratio of telodendrimer to lipid (weight:weight) within the cell free reaction mix is 0.1-10 with 0.5-2 preferred, and the ratio (weight/weight) of telodendrimer to polynucleotide within the cell free mix is between 10⁻⁷ and 0.1.

In particular, in embodiments herein described, the Immuno-t-NLPs such as CAR-t-NLPs can be formed by a lipid bilayer confined in a discoidal configuration by a scaffold protein and a telodendrimer. In this configuration, the lipid bilayer confined by the scaffold protein can be 3-6 nanometers in thickness, the nanolipoprotein particle can have an overall diameter between 5 nm to 100 nm in diameter and in particular a diameter of 25-50 nanometers, and the scaffold protein on the particle can have a thickness of 1-2 nanometers. In some embodiments, an entire NLP structure can be up to 600 kilodaltons in molecular weight.

The term “telodendrimer” refers to a dendrimer containing a hydrophilic covalently attaching a tail group T which comprises a hydrophilic polymer having a weight averaged molecular weight from 1 to 100 kDa. The term “attach” or “attached” as used herein, refers to connecting or uniting by a bond, link, force or tie in order to keep two or more components together, which encompasses either direct or indirect attachment where, for example, a first molecule is directly bound to a second molecule or material, or one or more intermediate molecules are disposed between the first molecule and the second molecule or material.

The term “dendrimers” used herein refer to repetitively branched molecules having three basis architectural components, namely (i) a focal point or group on a dendrimer core, (ii) repetitive plurality of branched monomer units covalently linked to the dendrimer core, and (iii) a plurality of end groups each covalently linked to a terminal monomer of the plurality of branched monomer units. In particular, a “dendrimer core” is a chemical moiety presenting a backbone and at least two anchor atoms, each anchor atom defining a bonding position to a head attachment atom of a branched monomer units.

In some embodiments, the dendrimer core can be formed by a branched monomer unit, for example, a lysine unit.

The term “monomer unit” or “monomer” in the sense of the disclosure is a chemical structure presenting one head attachment atom and at least one tail attachment atom. The head attachment atom defines a bonding position to an anchor atom of a dendrimer core or a tail attachment atom of another monomer unit. The tail attachment atom defines a bonding position to a head attachment atom of another branch cell unit or to a terminal functional group with the attachment possibly performed directly or indirectly.

A “branched monomer unit”, or “branched monomer”, is a monomer unit having at least two tail attachment atoms as also indicated. A generation of branched monomer unit within a dendrimer defines a shell of the dendrimer as will be understood by a skilled person (see “Dendrimers and other Dendritic polymers” by Jean M. J. Frechet and Donald A. Tomalia 2001.

[47] herein incorporated by reference in its entirety). The branched monomer unit of a generation typically define an interior space inside the dendrimer herein also indicated as interior of shell as will be understood by a skilled person. An “end group” of a dendrimer is a functional group or a chemical moiety presented on the outermost part of the dendrimer attached to an end of the branched monomer unit. The branched monomer unit attaching the end groups typically provide the outer shell or periphery of the dendrimer.

In the dendrimer core, the backbone of the dendrimer core can be any stable chemical moiety having the capability to present anchoring positions for the attachment of branched monomer units and a focal point for attachment to a linker moiety L, a spacer moiety A, or a tail group T.

In particular, the core backbone structure can be one of aromatic, heteroaromatic rings, aliphatic, or heteroaliphatic rings or chains. In some embodiments, the backbone of the dendrimer core can be one single atom, including C, N, O, S, Si, or P.

In a dendrimer as described herein, the branched monomer unit is linked together to form arms (or “dendrons”) extending from the focal point and terminating at the end groups. The focal point of the dendritic polymer can be attached to other segments of the telodendrimers, and the end groups may be further functionalized with additional chemical moieties.

In some embodiments herein described, one or more telodendrimers can be added to the lipid mixture before the mixing. In some embodiments, the ratio of lipid to telodendrimer to be added during the assembly process is 1:1 (W/W) to 1:100 (W/W), preferably 1:5 (W/W). In some embodiments, the ratio of DNA encoding CAR to DNA encoding scaffolding protein is between 1:1 (W/W) to 200:1 (W/W). Preferably, the ratio of lipid to telodendrimer to be added during the assembly process is 10:1 (W/W). Preferably, the ratio of DNA encoding CAR to DNA encoding scaffolding protein is between 5:1 to 50:1, more preferably between 10:1 to 25:1.

In some embodiments, the method can comprise addition of PEGSKCA8 telodendromer to the reaction mix at preferably at around 0.2 mg/ml, expandable to 0.02 mg/ml to 2 mg/ml. Resulting scaffold protein to telodendrimer mass ratio can be 15:1 to 1:1, preferably 5:1.

In some embodiments, scaffold protein to lipids mass ratio can be 1.5:1 to 0.1:1, preferably 0.5:1. In some embodiments, scaffold protein to lipids mass ratio will be reduced when CAR is inserted and may be altered to 1.5:0.75 to 0.1:0.75, preferably 0.5:0.75

In some embodiments, telodendrimers concentrations can be optimized for an immune cell transmembrane protein such as CAR by mixing them with lipids at concentrations from 0.5 to 10 mg (telodendrimer) and 5 to 60 mg (lipid) per mL. In some embodiments, the telodendrimer and lipid concentration can be at a 2 mg (telodendrimer) and 20 mg (lipid) per mL prior to addition to the cell-free reaction.

In some embodiments, the methods and systems herein described are performed at predefined lipid protein ratio, assembly conditions and/or with the use of preselected protein component (formed by immune cell transmembrane protein such as CAR and scaffold protein as polynucleotide) and lipid component (formed by Lipid and telodendrimers) so as to increase the yield, control the size and composition of the resulting NLP, provide an NLP of pre-determined dimensions, achieve desired functionality of the NLP, such as a certain level of loading capacity for a target molecule. In some embodiments, the molar ratio of lipid component to scaffold protein component is 10:1, 20:1, 30:1, 40:1, 50:1, 60:1, 70:1, 80:1, 90:1, 100:1, 110:1, 120:1, 130:1, 140:1, 150:1, 160:1, 170:1, 180:1, 190:1, 200:1, 210:1, 220:1, 230:1, and 240:1. In NLPs herein described, the lipid to scaffold protein component ratio can be determined on a case by case basis in view of the experimental design as will be understood by a skilled person.

In some embodiments, the mixing can comprise mixing one or more additives with the one or more membrane forming lipids, the polynucleotide coding for the CAR or other immune cell transmembrane protein, and the polynucleotide coding for the scaffold protein to provide a single reaction mixture further comprising the additive.

In particular in some embodiments, the additive can comprise one or more redox reagents incorporated into the reaction mix to improve solubility of the CAR protein or other immune cell transmembrane proteins. For example, use of glutathione or chaperonins can improve solubility of Immuno-NLPs slightly.

In some embodiments, the method to assemble Immuno-NLPs herein described results in modifying transmembrane protein synthesis such as CAR to an achieve improved transmembrane protein solubility. In particular, solubility can be measured by centrifuging the total cell-free mixtures following completion of the cell-free reaction (e.g., by a microcentrifuge at max speed for about 10 minutes). For example, in embodiments where the immune cell transmembrane protein is CAR, after CAR, the supernatant is collected and CAR solubility calculated as ratio of the amount CAR protein in supernatant to the amount of CAR protein in the total mixture. A percentage solubility can then be determined by calculating the amount of the CAR present in the supernatant (e.g., in term of molar concentration or mass concentration. Alternatively, dynamic light scattering or nanoparticle tracking methods can be used to assess particle size and aggregation state.

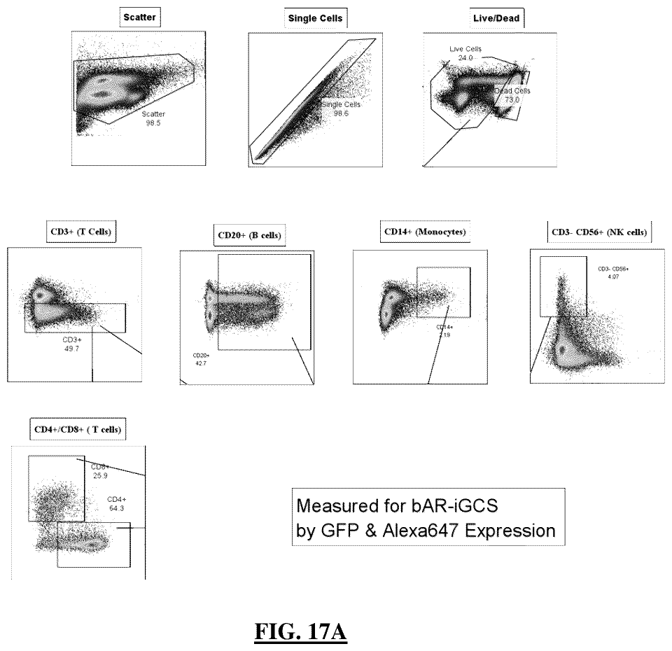

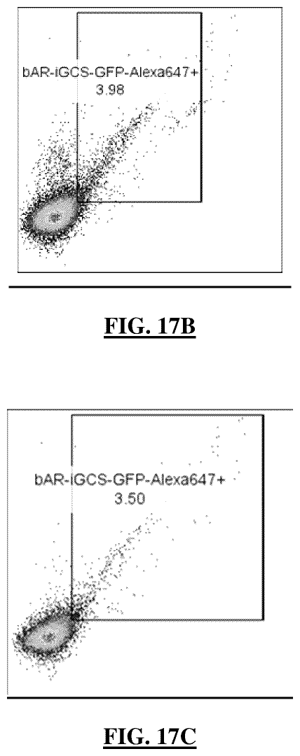

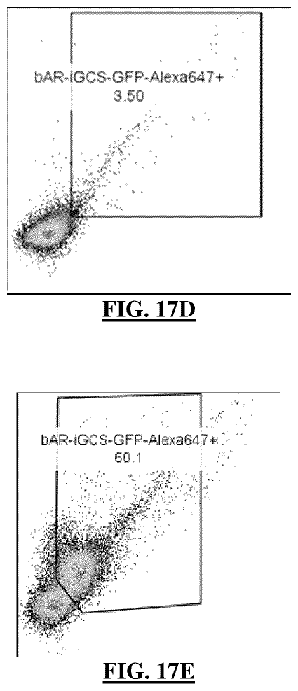

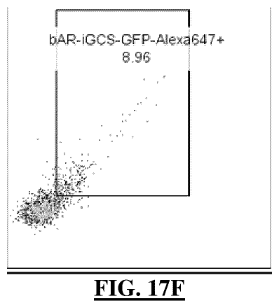

In some embodiments, a protein, which can form a structure associated with a desired functionality, such as an ARD, can be anchored to an NLP by means of conjugation to lipid moieties either through natural mechanisms of decorating proteins with lipid moieties, e.g., palmytolation or myrstolation, or incorporating into NLPs modified lipids capable of binding a protein. Either method generates a protein-NLP conjugate vehicle which the inventors demonstrate can be used for protein delivery to cells. For example, NLPs formed with modified lipids can be reacted with modified proteins that can form an antigen binding domain. The inventors demonstrate that a his-tagged scFv or a his-tagged GFP mixed with a nickel chelating NLP generates a noncovalent linkage [48, 49]. This conjugate can be used to deliver a protein or an ARD to the surface of an immune cell (Example 8, FIGS. 17A to 17F).

In preferred embodiments of the Immuno-NLP one-pot method, the incubating can be performed at a temperature from 18° C. to 22° C. and most preferably at 19.5° C. to 20.5° C. to provide an Immuno-NLP of the disclosure which is >50% soluble in buffered aqueous solutions.

In some embodiments, Immuno-NLPs of the disclosure can be manufactured by an Immuno-NLP two-pot method and system are described for producing a nanolipoprotein particle containing an Immunoreceptor (Immuno-NLP), such as CAR (CAR-NLP), and an Immuno-NLP composition as disclosed.

The method involves mixing one or more membrane-forming lipids with an SCP-encoding polynucleotide that codes for a scaffold protein in a first pot cell-free reaction mixture to create an NLP cell-free reaction mixture. In this two-pot method, the resulting NLP cell-free reaction mixture contains
i) the SCP-encoding polynucleotide at a total polynucleotide concentration ranging from 0.1 μg/mL to 100 μg/mL, and
ii) the membrane-forming lipids at a total lipid concentration ranging from 0.1 mg/mL to 100 mg/mL.

The Immuno-NLP two-pot method further involves incubating the first pot cell-free reaction mixture for a specified time and under conditions that allow for the translation of the scaffold protein in the presence of the membrane-forming lipids. This process leads to the self-assembly of a nanolipoprotein particle within a discoidal membrane lipid bilayer formed by the membrane-forming lipids and stabilized by the scaffold protein, thereby producing an assembled NLP.

In some embodiments, incubating the first pot cell-free reaction mixture is performed at a temperature between 15° C. and 40° C. with 29° C. to 31° C. preferred.

In some embodiments, in which the Immuno-NLP comprises a telodendrimer, one or more telodendrimer can be included in the first pot cell-free reaction mixture, wherein the telodendrimer to cell-free reaction mix is between 0.01 mg/ml and 1 g/ml with preferred ratios of 10 mg/ml to 40 mg/ml as will be understood by a skilled person.

Additionally, the Immuno-NLP two-pot method includes mixing in a second pot cell-free reaction mixture, the assembled NLP obtained from the incubation with a first pot cell-free reaction mixture with an ICTM polynucleotide. This second pot cell-free reaction mixture comprises:

the NLPs formed in the first pot reaction at a concentration from 0.01 mg/ml to 10 mg/ml and an ICTM -encoding polynucleotide that codes for the immune cell transmembrane protein at a total polynucleotide concentration ranging from 0.1 μg/mL to 100 μg/mL.

In the second pot cell-free reaction mixture, the mixing is conducted for a specified time and under conditions that facilitate the expression and attachment of the immune cell transmembrane protein within the discoidal membrane lipid bilayer of the assembled NLP. The membrane lipid bilayer attaches the immunoreceptor protein through the interaction of a hydrophobic region of the immune cell transmembrane protein with the membrane lipid bilayer.

Accordingly, some embodiments of the Immuno-NLP two-pot method of the disclosure comprises first translating and assembling NLPs by incubating in a first pot cell-free reaction mixture an SCP encoding polynucleotide encoding for a scaffold protein polynucleotide in a concentration between 0.1 μg/mL to 100 μg/mL with one or more membrane forming lipids at a total lipid concentration of from 0.1 mg/ml to 100 mg/ml, in which telodendrimers can optionally be included, wherein a telodendrimer to cell-free reaction mix is between 0.01 mg/ml and 1 g/ml with preferred ratios of 10 mg/ml to 40 mg/ml. The translating and assembling of NLPs is typically performed by incubating the cell-free reaction mixture at a temperature from 15° C. and 40° C. with 29° C. to 31° C. preferred.

Accordingly, some embodiments of the Immuno-NLP two-pot method of the disclosure comprises first translating and assembling NLPs by incubating in a first pot cell free reaction mix.

In those embodiments, the Immuno-NLP two-pot method of the disclosure can also comprise then mixing NLPs formed in the first pot cell-free reaction mixture at a concentration from 0.01 mg/ml to 10 mg/ml to a second pot cell-free reaction mixture containing a ICTM encoding polynucleotide encoding for the immune cell transmembrane protein in a polynucleotide concentration from 0.1 μg/mL to 100 μg/m. In the second pot cell-free reaction mixture, the mixing is performed for a time and under conditions allowing the expression and attachment of the immune cell transmembrane protein within the discoidal membrane lipid bilayer of the assembled NLP from the incubating, the membrane lipid bilayer attaching the immune cell transmembrane protein through interaction of a hydrophobic region of the immune cell transmembrane protein with the membrane lipid bilayer.

In embodiments herein described, the method to assemble Immuno-NLPs and in particular CAR-NLPs herein described results in a 0.2 CAR to NLP protein insertion ratio with a minimum of 10 nanomolar CAR, and correct ligand specificity of the ABD, which cannot be achieved using previously described methods (Example 5 and FIG. 8). Same and even higher ratios are expected to be obtained for immune transmembrane proteins other than CAR as will be understood by skilled person upon reading of the present disclosure.

In particular, in embodiments herein described, the Immuno-NLPs produced by the assembly method of the disclosure includes a CAR-nanolipoprotein particle (CAR-NLP) that comprises one or more membrane forming lipids, a scaffold protein and a chimeric antigen receptor (CAR) which comprise an antigen recognition domain, a hydrophobic transmembrane domain and an intracellular domain wherein the one or more membrane forming lipids are arranged in a discoidal membrane lipid bilayer stabilized by the scaffold protein, with the membrane lipid bilayer attaching the CAR thereof through interaction of the CAR transmembrane domain with the membrane lipid bilayer. In this configuration, techniques to purify NLPs also isolate CAR protein indicating correct association of these materials.

CAR-NLP produced by the assembly method of the disclosure can demonstrate functionality and correct protein folding. Accordingly, since CARs are representative of the ICTM the immune-NLPs of the disclosure and related compositions methods and systems herein and related method are known or expected to allow not only the expression, and solubilization, but also functional presentation of structurally complex, poorly soluble ICTMs using cell-free NLP platforms.

In particular, in some embodiments embedding single-pass ICTM proteins such as chimeric antigen receptors (CARs), cytokine receptors (e.g., CD127), and Fc receptors (e.g., CD64) into discoidal lipid scaffolds, the system is known or expected to result into providing the single-pass ICTMs within a biophysically appropriate environment for co-translational folding, membrane integration, and ligand recognition which will result into a functional single-pass ICTM.

The term “functional” as used herein indicates in connection with ICTM and in particular representative example CARs refers to a membrane-associated protein that following NLPs assembly retains at least one essential biological, biochemical, or binding activity characteristic of the native or intended functional form, associated with correct folding into a native-like tertiary structure.

In embodiments herein described, demonstrating functionality of an intercellular communication and trafficking molecule (ICTM) within the meaning of the present disclosure can be accomplished by a range of analytical methods that are appropriately matched to the structure and biological role of the ICTM. These methods fall into three principal categories: structural analyses, biochemical binding assays, and cellular functional assays. Each category provides independent and complementary evidence that the ICTM retains its native conformation, binding specificity, and/or signaling capacity when expressed, delivered, or incorporated into nanolipoprotein particles (NLPs) or other delivery vehicles as described herein. A functional ICTM, as contemplated in the disclosure, is one that exhibits behavior-whether structural, biochemical, or cellular—that is qualitatively and/or quantitatively consistent with its native form in the appropriate cellular context.

In some embodiments herein described structural functionality can be demonstrated using conformation-specific antibody binding assays that discriminate between folded and misfolded forms, or by physical characterization techniques such as small-angle X-ray scattering (SAXS), dynamic light scattering (DLS), electron microscopy (EM), or atomic force microscopy (AFM), which can confirm particle formation, oligomeric state, or conformational transitions associated with functional activity.

In some embodiments herein described, biochemical assays can be employed to show correct molecular interactions characteristic of the ICTM's native function. These include, without limitation, binding assays such as biolayer interferometry (BLI), surface plasmon resonance (SPR), enzyme-linked immunosorbent assays (ELISA), and dot blot or pull-down assays for recognition of known ligands or interacting proteins. Where appropriate, signal transduction can be monitored via biochemical indicators such as phosphorylation of downstream proteins, conformational shifts within the receptor itself (e.g., dimerization), or release of small molecules such as GTP in the case of G protein-coupled receptors (GPCRs). Western blotting or phospho-specific flow cytometry can be used to detect activation-related post-translational modifications. For example, a functional T cell receptor (TCR) can be shown to associate with CD3ζ chains and trigger downstream phosphorylation events in response to peptide-MHC engagement, whereas a GPCR can be shown to associate with its cognate G protein and catalyze nucleotide exchange.

In some embodiments herein described, cell-based assays further allow functional validation in a context where the ICTM is expressed or delivered into recipient cells lacking endogenous expression of the molecule. These assays can demonstrate correct localization of the ICTM to the plasma membrane, intracellular organelles, or other compartments, using immunofluorescence, confocal microscopy, or flow cytometry. Colocalization with known binding partners, as assessed by proximity ligation assay or co-immunoprecipitation, further supports proper folding and integration. Functional responsiveness can be assessed by exposing recipient cells to the ligand or stimulus of interest and detecting a measurable phenotypic change. Such changes can include upregulation of activation markers (e.g., CD69, CD25), cytokine secretion, cell proliferation, calcium flux, or cytotoxic responses, depending on the nature of the ICTM.

In some exemplary embodiments, in the case of a chimeric antigen receptor (CAR) specific for CD19, functionality can be established by demonstrating binding of the CAR to CD19 protein in vitro and, in a cellular context, by showing that delivery of the CAR protein to cells lacking endogenous CAR expression results in enhanced binding to CD19-expressing target cells, as determined by flow cytometry. Furthermore, delivery of CAR protein to T cells followed by stimulation with CD19 protein or CD19-expressing target cells can result in T cell activation, as evidenced by increased expression of CD69 or CD25, cytokine production measured by ELISPOT or intracellular staining, and cytotoxicity against CD19+targets in co-culture assays.

In some embodiments where the ICTM comprise cytokine receptors, functionality can similarly be demonstrated by ligand-binding assays confirming affinity for the cognate cytokine, observation of receptor dimerization or conformational changes upon ligand exposure, and downstream phosphorylation events or transcriptional activation measured by western blot, phospho-flow, or reporter assays. Cellular functionality can be confirmed by showing that receptor-treated cells change their phenotype or signaling profile in response to cytokine stimulation.

The disclosure further contemplates use of both experimental and prophetic examples employing multi-donor peripheral blood mononuclear cells (PBMCs) to validate the generalizability of the ICTM function across human genetic backgrounds. Flow cytometry-based assays are suitable for detecting surface localization of ICTMs and upregulation of immune activation markers such as CD69 and CD25, in response to defined stimuli. Such approaches can be used to compare the responsiveness of NLP-delivered ICTMs to that of conventionally expressed receptors in native immune cells, thereby confirming functional equivalence.

Collectively, these methodologies provide a robust framework for confirming that the ICTMs included in Section II of the disclosure retain their functional integrity after cell-free synthesis, NLP incorporation, and delivery to target cells. This framework is broadly applicable across receptor types, including but not limited to CARs, cytokine receptors, pattern recognition receptors, co-stimulatory molecules, and adhesion molecules, and can be extended to any ICTM for which a biologically relevant output can be identified and measured.

In some embodiments, functional ICTM proteins and in particular functional single-pass ICTM protein are expected to be used for immune cell differentiation, immune cell redirection, phenotypic modulation, diagnostic screening, or therapeutic cell programming, or for engineering immune cells responsive to non-native ligands, with applications in adoptive cell therapy, immuno-oncology, and synthetic immunology as will be understood.

The concentration of CAR protein within CAR-NLPs in this disclosure is at least 0.2 CAR: NLP molar ratio and a minimum of 17.5 nanomolar CAR protein. This range is expandable to a theoretical maximum of 5CAR: NLP, and 1milimolar CAR (for 600 kDa max weight for an ApoA1 NLP+theoretical solubility of NLP).

Membrane forming lipid components for CAR-NLPs in this disclosure comprises at least one phospholipid, selected from soy phosphatidylcholine, egg phosphatidylcholine, soy phosphatidylglycerol, cgg phosphatidylglycerol, palmitoyl-olcoyl-phosphatidylcholinc distcaroylphosphatidylcholine, distcaroylphosphatidylglycerol phosphatidylcholine, phosphatidylglycerol, sphingomyelin, phosphatidylserine, phosphatidic acid, phosphatidylethanolamine, lysolecithin, lysophosphatidylethanolamine, phosphatidylinositol, cephalin, cardiolipin, cerebrosides, dicetylphosphate, diolcoylphosphatidylcholine, dipalmitoylphosphatidylcholine, dipalmitoylphosphatidylglycerol, diolcoylphosphatidylglycerol, stearoyl-palmitoyl-phosphatidylcholine, di-palmitoyl-phosphatidylethanolamine, distearoyl-phosphatidylethanolamine, di-myrstoyl-phosphatidylserine, di-myrstoyl-phosphatidylcholine and diolcyl-phosphatidylcholine. Lipid components can be expanded to any lipid combination capable of forming a lipid bilayer. Preferred lipids include DMPC, DMPG, POPC, and combinations thereof.

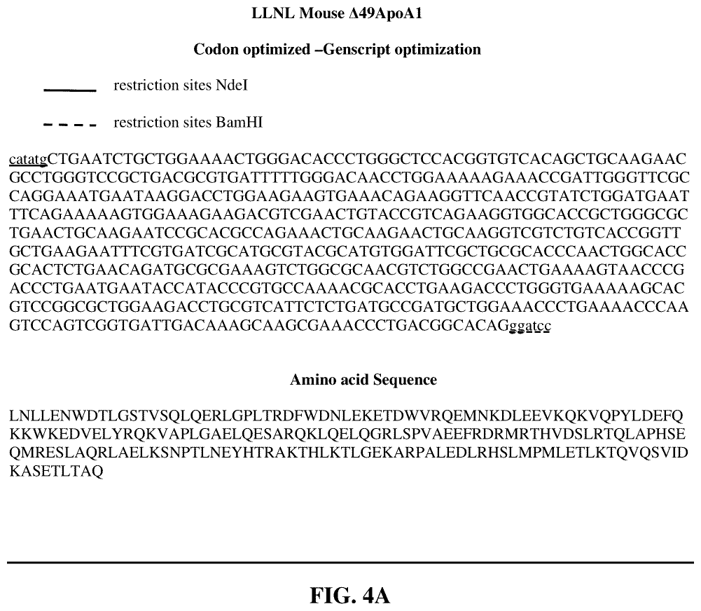

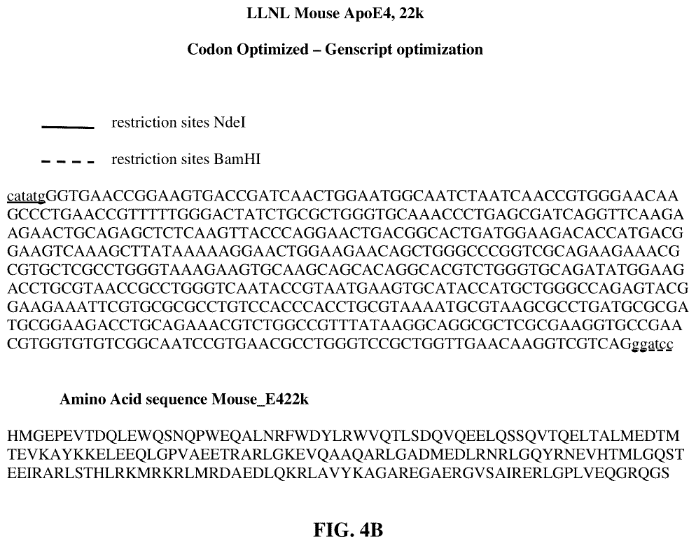

Scaffold protein components include amphipathic proteins found in lipoproteins from mammalian species and similar proteins capable of scaffolding a NLP, such as one or more of a human derived apoE4, a truncated versions of human derived apoE4, a human derived apoE3, a truncated versions of human derived apoE3, a human derived apoE2, a truncated versions of human derived apoE2, a human derived apoAl, a truncated version of human derived apoAl, a mouse derived apoE4, a truncated version of mouse derived apoE4, mouse derived apoE3, truncated versions of mouse derived apoE3, a mouse derived apoE2, a truncated version of mouse derived apoE2, a mouse derived apoA 1, a truncated versions of mouse derived apoA1, a rat derived apoE4, a truncated version of rat derived apoE4, a rat derived apoE3, a truncated versions of rat derived apoE3, a rat derived apoE2, a truncated version of rat derived apoE2, a rat derived apoAl, a truncated versions of rat derived apoA1, a lipophorins, a synthetic cyclic peptide mimicking an apolipoprotein function. Preferred scaffold proteins include truncated versions of ApoA1 and ApoE4 (see e.g., Example 4, FIGS. 4A, 4B and FIGS. 5 to 7).

In some embodiments, the scaffold protein can be codon-optimized in order to improve protein expression in expression systems of a particular organism. Exemplary polynucleotide and amino acid sequences of E. coli codon optimized scaffold protein are shown in Example 4 and FIGS. 4A and 4B. Exemplary polynucleotide sequences of E. coli codon optimized scaffold protein are shown in FIGS. 5 to 7.

In some embodiments, the scaffold protein is formed by amphipathic peptides and/or synthetic apolipoproteins, which are configured to maintain an amphipathic structure and capability of self-assembly. In particular, in those embodiments, the peptides and/or synthetic apolipoprotein are configured and selected to provide the a plurality of helical segments each having a primary structure configured to form an alpha helix secondary structure, In the alpha helix secondary structure of at least one helical segment, the peptides and/or synthetic apolipoprotein comprise a plurality of hydrophobic amino acids and a plurality of hydrophilic amino acids positioned in the primary structure to provide an amphipathic alpha helix secondary structure, with the plurality of hydrophobic amino acids forming an hydrophobic amino acid cluster and the plurality hydrophilic amino acids forming an hydrophilic amino acid cluster. In some of those embodiments, the scaffold proteins can be peptides derived from apolipoproteins, and can contain amino acid additions, deletions, or substitutions. In other embodiments, these peptides have no sequence homology to apolipoproteins but can be structural analogs. In some embodiments, the peptides can be prepared with L- or D-amino acids. In embodiments where the scaffold protein comprises one or more peptides the skilled person would be able to identify the ratios of peptides based on the length and number of peptides and apolipoproteins and on a desired dimension of the nanolipoprotein particles upon reading of the present disclosure. Additional description of scaffold proteins can be found in PCT/US2015/051172 published on Mar. 16, 2017, as WO2017/044899, incorporated herein by reference in its entirety.

In embodiments herein described further comprising telodendrimers, the dendritic polymer can be any suitable dendritic polymer. The dendritic polymer can be made of branched monomer units including amino acids or other bifunctional XY2 type monomers, where X and Y are two different functional groups capable of reacting together such that the resulting polymer chain has a branch point where an X-Y covalent bond is formed. For example, in the case of lysine, when X is a carboxylic acid and Y is an amino group, an amide bond can be form between X and Y. In some embodiments, each branched monomer unit X can be a diamino carboxylic acid, a dihydroxy carboxylic acid and a hydroxyl amino carboxylic acid.

In some embodiments, each diamino carboxylic acid can be 2,3-diamino propanoic acid, 2,4-diaminobutanoic acid, 2,5-diaminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminocthyl) butyric acid or 5-amino-2-(3-aminopropyl) pentanoic acid. In some embodiments, each dihydroxy carboxylic acid can be glyceric acid, 2,4-dihydroxybutyric acid, 2,2-Bis(hydroxymethyl) propionic acid, 2,2-Bis(hydroxymethyl) butyric acid, serine or threonine.

In some embodiments, each hydroxyl amino carboxylic acid can be serine or homoserine. In some embodiments, the diamino carboxylic acid is an amino acid. In some embodiments, each branched monomer unit X is lysine.

The dendritic polymer of the telodendrimer can be any suitable generation of dendrimer, including generation 1, 2, 3, 4, 5, or more, where each “generation” of dendrimer refers to the number of branch points encountered between the focal point and the end group following one branch of the dendrimer. The dendritic polymer of the telodendrimer can also include partial-generations such as 1.5, 2.5, 3.5, 4.5, 5.5, etc., where a branch point of the dendrimer has only a single branch. The various architectures of the dendritic polymer can provide any suitable number of end groups, including, but not limited to, 2, 3, 4, 5, 6, 7, 8, 9, 10, 11, 12, 13, 14, 15, 16, 17,18, 19, 20, 21, 22, 23, 24, 25, 26, 27, 28, 29, 30, 31 or 32 end groups.

The telodendrimer backbone can vary, depending on the number of branches and the number and chemical nature of the end groups and R groups, which will modulate solution conformation, rheological properties, and other characteristics. The telodendrimers can have any suitable number n of end groups and any suitable number of R groups. In some embodiments, n can be 2-70, or 2-50, or 2-30, or 2-10. In some embodiments, n is 2-20.

The R groups installed at the telodendrimer periphery can be any suitable chemical moiety, including, for example, hydrophilic groups, hydrophobic groups, or amphiphilic compounds. Examples of hydrophobic groups include, but are not limited to, long-chain alkanes and fatty acids, fluorocarbons, silicones, certain steroids such as cholesterol, and many polymers including, for example, polystyrene and polyisoprene. Examples of hydrophilic groups include, but are not limited to, alcohols, short-chain carboxylic acids, amines, sulfonates, phosphates, sugars, and certain polymers such as PEG. Examples of amphiphilic compounds include, but are not limited to, molecules that have one hydrophilic face and one hydrophobic face.

Amphiphilic compounds that can be used in the preparation of CAR-NLPs herein described comprise cholic acid and cholic acid analogs and derivatives. “Cholic acid” refers to (R)-4-((3R,5S,7R,8R,9S,10S,12S, 13R, 14S, 17R)-3,7, 12-trihydroxy-10, 13 dimethylhexadecahydro-1H-cyclopenta[a]phenanthren-17-yl) pentanoic acid. Cholic acid derivatives and analogs comprise allocholic acid, pythocholic acid, avicholic acid, deoxycholic acid, and chenodeoxycholic acid. Cholic acid derivatives can be designed to modulate the properties of the nanocarriers resulting from telodendrimer assembly, such as micelle stability and membrane activity. For example, the cholic acid derivatives can have hydrophilic faces that are modified with one or more glycerol groups, aminopropanediol groups, or other groups.

In some embodiments, each R of the telodendrimer of Formula (I) can be cholic acid, (3α,5 (3,70α, 12α)-7,12-dihydroxy-3-(2,3-dihydroxy-1-propoxy)-cholic acid, (3α,5β, 70α, 12α)-7-hydroxy-3,12-di(2,3dihydroxy-1-propoxy)-cholic acid, (3α,5β, 7α, 12α)-7,12-dihydroxy-3-(3-amino-2-hydroxy-1-propoxy)-cholic acid, cholesterol formate (CF), doxorubicin, or rhein. In some embodiments, each amphiphilic compound is cholic acid (CA). In some embodiments, each amphiphilic compound is cholesterol formate (CF).

In some embodiments, the tail group T can be a moiety of Formula (XI)

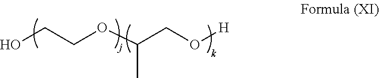

wherein i and j can be independently selected from 2-3000, preferably 22-2300, and more preferably 22-230; and
wherein the polymer of Formula (XI) can be attached by way of any one of the two terminal hydroxyl groups to an end group of the dendrimer.

In some embodiments, i and j together can be independently selected from 2-3000, preferably 22-2300, and more preferably 22-230. In some embodiments, the tail group can be polyethylene glycol, PEG, (k=0 in Formula (XI)), polypropylene glycol (j=0 in Formula XI) or a polyethylene-b-polypropylene glycol (j>0, k>0) in Formula (XI).

In some embodiments herein described, the telodendrimers herein described are block copolymers having a linear poly(ethylene glycol)(PEG) moiety and a dendritic hydrophobic segment or a dendritic amphiphilic moiety. Telodendrimers can also have additional functional groups such as cholic acid groups and hydrophobic groups (e.g. hydrophobic moieties with drug properties) covalently bound to the dendritic segment.

As used herein, the term “hydrophobic group” refers to a chemical moiety that is water-insoluble or repelled by water. Examples of hydrophobic groups include, but are not limited to, C1-C4 short-chain alkanyls, C5-C22 long-chain alkanyls, C1-C4 short-chain alkenyls, C5-C22 long-chain alkenyls, C1-C4 short-chain alkynyls, C5-C22 long-chain alkenyls and fatty acids, fluorocarbons, silicones, certain steroids such as cholesterol, and many polymers including, for example, polystyrene and polyisoprene or their derivatives.

As used herein, the term “hydrophilic group” refers to a chemical moiety that is water-soluble or attracted to water. Examples of hydrophilic groups include, but are not limited to, alcohols, short-chain carboxylic acids, quaternary amines, sulfonates, phosphates, sugars, and certain polymers such as poly(ethylene glycol)(PEG).

In some embodiments, the PEG as used herein can have 2 to 3000 ethylene glycol units, —(CH₂CH₂O)—, preferably 22-2300 ethylene glycol units, and more preferably 22-230 ethylene glycol units.

It is also to be understood that, unless otherwise specified herein, the molecular weight of a polymer herein refers to a weight average molecular weight. In the instant disclosure molecular weight of a polymer, e.g., PEG can be indicated as a superscript together with the indication of the polymer (e.g., a PEG of 2000 DA can also be indicated as PEG2k)

As used herein, the term “amphiphilic compound” or “amphiphilic moiety” refers to a compound or moiety having both hydrophobic portions and hydrophilic portions. For example, the amphiphilic compounds herein described can have one hydrophilic face of the compound and one hydrophobic face of the compound.

In some embodiments, in telodendrimers of the disclosure, the tail group T is attached to the dendrimer through a spacer A and/or a linker L.

As used herein the term “spacer A” indicates a spacer moiety formed by one or more monomers configured to be directly covalently connected to one or more tail groups T and to one linker moiety L.

As used herein, the term “linker” or “linker moiety” refers to a chemical moiety formed by one or more monomers configured to be directly covalently bonded to a spacer A and a focal point of a dendrimer. The types of bonds used to link the linker L to the focal point of the dendrimer D and the spacer A include, but are not limited to, amides, amines, esters, carbamates, ureas, thioethers, thiocarbamates, thiocarbonates and thioureas and additional bonds as will be understood by a skilled person.

In particular, in some embodiments, the telodendrimer of the present disclosure can have a general Formula (I):

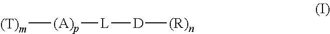

wherein
D is a dendrimer
T is a tail group;
A is a spacer moiety configured to be directly covalently connected to each T and to a linker moiety L, and comprises a polymer of 1 to m number of spacer A monomers, wherein the spacer A monomer comprises a substituted or unsubstituted linear C1-C15 alkyl; branched C3-C15 alkyl; cyclic C3-C15 alkyl; linear, cyclic, or branched C2-C15 alkenyl; linear, cyclic, or branched C2-C15 alkynyl; C6-C20 substituted or unsubstituted aryl; and C6-C20 substituted or unsubstituted heteroaryl.
m is 0-20 and p is 0-1, and
   wherein m is 0 or 1 when p is 0; or m is 2-20 when p is 1.

In some embodiments, L can be a polymer of 1 to m number of independently selected spacer A monomers, wherein the spacer A monomer comprises a substituted or unsubstituted linear C1-C15 alkyl; branched C3-C15 alkyl; cyclic C3-C15 alkyl; linear, cyclic, or branched C2-C15 alkenyl; linear, cyclic, or branched C2-C15 alkynyl; C6-C20 substituted or unsubstituted aryl; and C6-C20 substituted or unsubstituted heteroaryl, wherein each branch of the dendrimer is adapted to present an end group R by a covalent bond.

In some of those embodiments, each end group R is independently a hydrophobic group, a hydrophilic group, an amphiphilic group, H, or a functional group such as halo, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including for example C₂-C₂₄ alkylcarbonyl (—CO-alkyl) and C₆-C₂₄ arylcarbonyl (—CO-aryl)), acyloxy (—O-acyl, including for example C₂-C₂₄ alkylcarbonyloxy (—O—CO-alkyl) and C₆-C₂₄ arylcarbonyloxy (—O—CO-aryl)), C₂-C₂₄ alkoxycarbonyl (—(CO)—O-alkyl), C₆-C₂₄ aryloxycarbonyl (—(CO)—O-aryl), C₂-C₂₄ alkylcarbonato (—O—(CO)—O-alkyl), C₆-C₂₄ arylcarbonato (—O—(CO)—O-aryl), carboxy (—COOH), carboxylato (—COO″), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl) ₂), mono-(C₅-C₂₄ aryl)-substituted carbamoyl (—(CO)—NH-aryl), di-(C₅-C₂₄ aryl)-substituted carbamoyl (—(CO)—N(C₅-C₂₄ aryl) ₂), di-N-(C₁-C₂₄ alkyl),N-(C₅-C₂₄ aryl)-substituted carbamoyl, thiocarbamoyl (—(CS)—NH₂), mono-(C₁-C₂₄ alkyl)-substituted thiocarbamoyl (—(CO)—NH(C₁-C₂₄ alkyl)), di-(C₁-C₂₄ alkyl)-substituted thiocarbamoyl (—(CO)—N(C₁-C₂₄ alkyl) ₂), mono-(C₅-C₂₄ aryl)-substituted thiocarbamoyl (—(CO)—NH-aryl), di-(C₅-C₂₄ aryl)-substituted thiocarbamoyl (—(CO)—N(C₅-C₂₄ aryl)₂), di-N—(C₁-C₂₄ alkyl),N—(C₅-C₂₄ aryl)-substituted thiocarbamoyl, carbamido (—NH—(CO)—NH₂), cyano (—C≡N), cyanato (—O—C≡N), thiocyanato (—S—C≡N), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), C₂-C₂₀ alkylimino (—CR═N (alkyl), where R=hydrogen, C₁-C₂₄ alkyl, C₅-C₂₄ aryl, C₆-C₂₄ alkaryl, C₆-C₂₄ aralkyl, etc.), arylimino (—CR═N (aryl), where R═ hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO2-aryl), boryl (—BH2), borono (—B(OH) 2), boronato (—B(OR)₂ where R is alkyl or other hydrocarbyl), phosphono (—P(O)(OH)₂), phosphonato (—P(O)(O″)₂), phosphinato (—P(O)(O″)), phospho (—PO₂), phosphino (—PH₂), silyl (—SiR₃ wherein R is hydrogen or hydrocarbyl), and silyloxy (—O-silyl); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

In some embodiments, the tail group T is polyethyleneglycol (PEG) polymers, with each of the m number PEG polymer independently having a weight average molecular weight of 1-100 kDa.

In some embodiments, a telodendrimer can have the at least one tail group T having polyethyleneglycol (PEG) polymer moiety, a dendritic polymer moiety D, and at least one end group R which includes but is not limited to a hydrophobic group, a hydrophilic group, an amphiphilic compound or a drug on the dendrimer periphery or branch, wherein the dendritic polymer moiety D has a single focal group and n number of branches.

In some embodiments, a telodendrimer can comprise one or more of the following monomers in combination within a dendrimer, spacer moiety A and/or linker moiety be XY2-type monomers, where X and Y are two different functional groups capable of reacting together such that the resulting polymer chain has a branch point where an X-Y bond is formed. Exemplary monomers include a diamino carboxylic acid, a dihydroxy carboxylic acid and a hydroxyl amino carboxylic acid. Examples of diamino carboxylic acid groups include 2,3-diamino propanoic acid, 2,4-diaminobutanoic acid, 2,5-diaminopentanoic acid (ornithine), 2,6-diaminohexanoic acid (lysine), (2-Aminocthyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. Examples of dihydroxy carboxylic acid groups include glyceric acid, 2,4-dihydroxybutyric acid, and 2,2-bis(hydroxymethyl) propionic acid. Examples of hydroxyl amino carboxylic acids include serine and homoserine. One of skill in the art will appreciate other monomer units useful in the current disclosure.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

In some embodiments, in Formula (I), subscript n is an integer from 2 to 128, wherein subscript n is equal to the number of end group R, wherein each end group R is covalently linked to the dendritic polymer D, and wherein at least half the number n of R groups are each independently a hydrophobic group, a hydrophilic group, an amphiphilic group or a drug.

In Formula (I), subscript p can be 0 or 1, wherein when p is 0, m can be 0 or 1; when p is 1, m can be 2 to 20, wherein each of the m number of PEG is directly covalently linked to A and each of the m number of PEGs is independently selected from a molecular weight of 1 to 100 kDa, or preferably a molecular weight of 1 kDa (PEG1000) to a molecular weight of 10 kDa (PEG 10,000).

In some embodiments, spacer moiety A can be a monomer or an oligomer presenting at least two tail groups. As used herein, the terms “monomer” and “monomer unit” for spacer moiety A refers to repeating units that make up the spacer moiety A herein described. The monomers may be XY2-type monomers, where X and Y are two different functional groups capable of reacting together such that the resulting polymer chain has a branch point where an X-Y bond is formed.

For purpose of making spacer moiety A, one of the two Y's of a XY2-type monomer can be orthogonally protected, for example by way of Fmoc (Fluorenylmethyloxycarbonyl), Boc (t-butyloxycarbonyl), or DDE ((4,4-dimethyl-2,6-dioxocyclohex-1-ylidene)ethyl) when B is an amino group and A is a carboxylic acid.

Therefore, each of the XY2 in spacer moiety A is capable of having a covalent bond with a tail group T.

Exemplary monomers for spacer moiety A include a diamino carboxylic acid, a dihydroxy carboxylic acid, and a hydroxylamino carboxylic acid. Examples of diamino carboxylic acid groups herein described comprise 2,3 diamino propanoic acid, 2,4-diaminobutanoic acid, 2,5-di aminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. Examples of dihydroxy carboxylic acid groups of telodendrimers of the present disclosure comprise glyceric acid, 2,4-dihydroxy butyric acid, and 2,2-bis(hydroxymethyl) propionic acid. Examples of hydroxylamino carboxylic acids include, but are not limited to, serine and homoserine as well as additional monomeric units as will be understood by a skilled person.

In some embodiments, spacer moiety A comprises an oligomer of lysine represented by (K) m′, wherein oligomer of lysine has a peptide backbone based on an alpha amino group of lysine, wherein K is lysine and m′ is 1-20 and wherein m″ is an integer between m-1 to 20. In some embodiments, m″ is m-1.

In some embodiments, at least one of the dendrimer, spacer moiety A and/or linker moiety L can independently comprise at least one monomer selected from XY2-type monomers, where A and B are two different functional groups capable of reacting together such that the resulting polymer chain has a branch point where an X-Y bond is formed. Exemplary monomers include a diamino carboxylic acid, a dihydroxy carboxylic acid and a hydroxylamino carboxylic acid. Examples of diamino carboxylic acid groups herein described comprise 2,3 diamino propanoic acid, 2,4-diaminobutanoic acid, 2,5-di aminopentanoic acid (omithine), 2,6-diaminohexanoic acid (lysine), (2-Aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. Examples of dihydroxy carboxylic acid groups of telodendrimers of the present disclosure comprise glyceric acid, 2,4-dihydroxy butyric acid, and 2,2-bis(hydroxymethyl) propionic acid. Examples of hydroxylamino carboxylic acids include, but are not limited to, serine and homoserine as well as additional monomeric units as will be understood by a skilled person.

In some embodiments a dendrimer can comprise branched polymers containing a focal point, a plurality of branched monomer units, and a plurality of end groups in which the focal point of the dendritic polymer is a functional group on the branched monomer that is of equal spacing from all the end groups and can be attached to another segment of the telodendrimer, including linker L, spacer A or tail group T. The end groups may be further functionalized with additional chemical moieties.

In embodiments wherein the telodendrimer has Formula (I), the focal point of a telodendrimer or a telodendrimer segment can be any suitable functional group that forms a covalent bond between the dendrimer and a tail group T, spacer moiety A, a linker moiety L.

In some embodiments, the functional group for the focal point can be a nucleophilic group including, but not limited to, an alcohol, an amine, a thiol, or a hydrazine. The focal point functional group can also be an electrophile such as an aldehyde, a carboxylic acid, or a carboxylic acid derivative including for example an acid chloride or an N-hydroxysuccinimidyl ester.

The telodendrimer of Formula (I) can have a single type of R group on the periphery, or any combination of R groups in any suitable ratio. In general, at least half the number n of R groups are other than an end group. For example, at least half the number n of R groups can be a hydrophobic group, a hydrophilic group, an amphiphilic compound, a drug, or any combination thereof. In some embodiments, half the number n of R groups are amphiphilic compounds.

In some embodiments, all the R groups are an amphiphilic group such as cholic acid or cholesterol formate. In other embodiments, some of the R groups are an end group of the dendrimer. In some other embodiments, at least two different R groups are present, such as two different amphiphilic groups, or an amphiphilic group and a drug, or an amphiphilic group and a dendritic polymer end group, or two different drugs, or a drug and a dendritic end group.

In some embodiments, telodendrimers of t-NLPs of Formula (I), D can be lysine, L can be a bond, R can be cholic acid or cholate, m can be 1, and/or n can be 2, 4 or 8. In some embodiments, R can be formed by a detergent moiety, a lipid and/or an amino acid such as HIS, GLU.

In some embodiments, the telodendrimer of the present disclosure comprise a compound of Formulas (II)-(III):

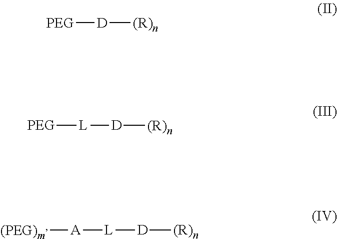

wherein D, L, R and n are as defined for formula (I) and subscript m′ of formula (IV) is 2-20.

In some embodiments, the PEG in telodendrimer of any one of Formula (I) to (IV) can be a PEG having a molecular weight from 1 kDA (PEG1000) to 10 kDA (PEG 10,000).

In some embodiments, CAR-t-NLPs herein described can comprise telodendrimers such as PEG^{2K}_D-CA4, PEGSK-D-CA4, PEG^{IOK}-D-CA4, PEG^{2K}-D-CA₈, PEG^{5K}-D-CA₈, PEG^{IOK}-D-CA₈, PEG^{2K}_D-CF₄, PEGSK-D-CF₄, PEG^{IOK}-D-CF₄, PEG^{2K}-D-CF₈, PEGSK-D-CF₈, or PEG^{IOK}-D-CF₈, wherein each dendritic polymer D is a poly(lysine) dendritic polymer wherein each end group is hydroxy. In one embodiment, the telodendrimer can be PEG^{5K}-D-CF₈. Additional modifications for the telodendrimer can include attachment of lipidic and detergent moieties such as Telo-His and Telo-Cys.

In some embodiments, CAR-t-NLPs herein described can comprise telodendrimers such as PEG^{2K}-D-CA₄, PEGSK-D-CA₄, PEG^{IOK}-D-CA₄, PEG^{2K}-D-CA₈, PEGSK-D-CA₈, PEG^{IOK}-D-CA₈, PEG^{2K}-D-CF₄, PEG^{5K}-D-CF₄, PEG^{IOK}-D-CF₄, PEG^{2K}_D-CF₈, PEGSK-D-CF₈, or PEG^{IOK}-D-CF₈, wherein each dendritic polymer D is a poly(lysine) dendritic polymer wherein each end group is hydroxy. In one embodiment, the telodendrimer can be PEG^{5K}-D-CF₈. Additional modifications for the telodendrimer can include attachment of lipidic and detergent moieties such as Telo-His and Telo-Cys (see e.g., Example 3).

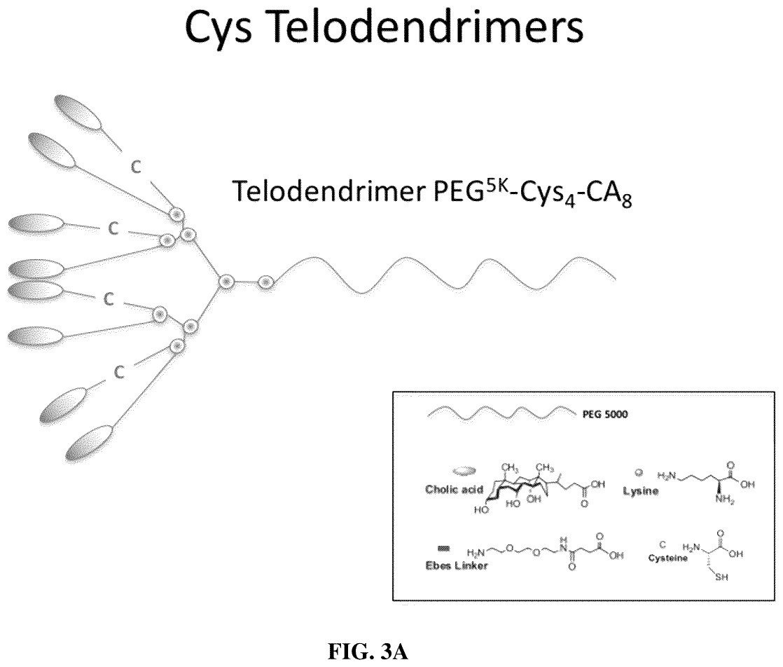

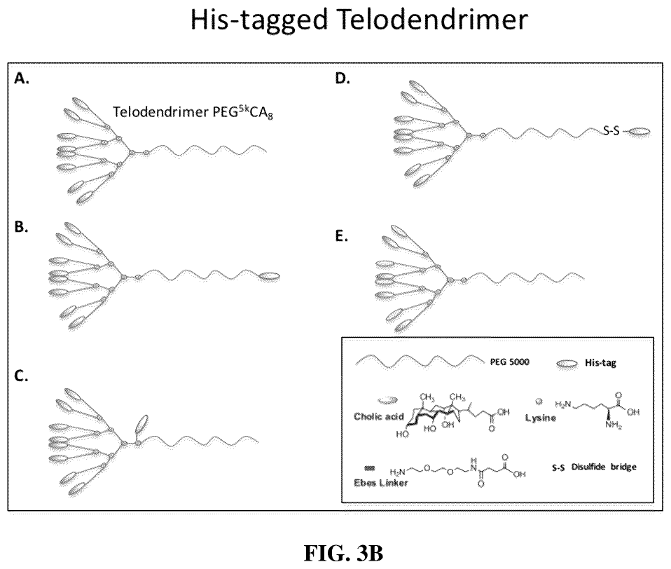

A discussion of exemplary Telo-Hi and Telo-cys is reported in Example 3, and a schematic representation of an exemplary telodendrimer comprising a telo-cys is shown in FIG. 3A. A schematic representation of an exemplary telodendrimer comprising telo-His is shown in FIG. 3B. In some embodiments, in a Telo-Cys according to the schematic representation of FIG. 3A, or in a Telo-His, the cholic acid is covalently linked to a cysteine amine or a lysine by amide bond; cysteine and lysine or lysine and lysine are covalently connected by an amide bond and a core lysine monomer is covalently attached to a tail group of PEG 5000. In some embodiments, telodendrimers herein described can comprise a combination of cysteine and histidine as will be understood by a skilled person.

In some embodiments, an Ebes linker, (N-(Fmoc-8-amino-3,6-dioxa-octyl) succinamic acid), is present between the tail group PEG 5000 and the core lysine monomer by amide bond and an ester bond.

In particular, in preferred embodiments, CAR-t-NLPs comprising one or more of PEG^{5K} D-CA₄, PEGSK-D-CA₈, PEGSK-D-CF₄, and PEGSK-D-CF₈, provided an improved formulation of MOMP proteins within a t-NLP as compared to other telodendrimers herein described. The telodendrimers useful in the preparation of t-NLPs herein described can be prepared by a variety of methods, such as those described in PCT Publication No. WO 2010/039496 herein incorporated by reference in its entirety.

In embodiments herein described, NLPs comprise scaffold protein and a lipid component comprising membrane forming lipids and possibly other lipids, as well as telodendrimers and CAR in ratios and proportions that would be identifiable by a skilled person upon reading of the present disclosure.

In general, assembly of t-NLPs can be accomplished with a wide range of ratios of total membrane forming lipids to scaffold proteins as previously described. Telodendrimer can be incorporated at a ratio of 1:10 to 1:1000 telodendrimer to lipid, with a preferred ratio between 1:50 and 1:500, or more preferably between 1:100 and 1:200.

The t-NLPs here described can contain any suitable combination of lipids with telodendrimers and/or other components. In particular, the one or more membrane forming lipids mixed to form a NLP can be polar and/or non-polar lipids as will be understood by a skilled person upon reading of the present disclosure. The telodendrimers mixed to form the NLPs can comprise PEG with lengths of 1000-10000 kDa. The ratio of lipid to telodendrimer in the NLPs, for example, can be from about 1000:1 to about 10:1 (mol/mol). For example, the ratio can be about 1000:1, 900:1, 800:1, 700:1, 600:1, 500:1, 400:1, 300:1, 200:1, 100:1, 99:1, 95:1, 90:1, 80:1, 75:1, 70:1, 60:1, 50:1, 40:1, 30:1, 25:1, 20:1, 15:1, 14:1, 13:1, 12:1, 11:1 or 10:1 (mol/mol) wherein the term about when referred to ratios indicates the ratios+5%. In some embodiments, the ratio of lipid to telodendrimer is from about 200:1 to about 100:1 (mol/mol). In some embodiments, the ratio of lipid to telodendrimer is about 150:1 (mol/mol). In some embodiments, the ratio of lipid to telodendrimer is about 135:1 (mol/mol). Other molar ratios of lipid to telodendrimer can also be useful in t-NLPs herein described as will be apparent to a skilled person upon reading of the present disclosure. In some embodiments of t-NLPs, the lipid to telodendrimer ratios within the t-NLPs herein described can be of 1000:1 to 10:1, preferably 500:1 to 50:1.

In some embodiments, a CAR-NLP herein described can have a ratio of CAR to scaffold protein of 50:1 to 1:10. In some embodiments, the ratio of CAR to scaffold protein can be 20:1 to 1:4, 5:1 to 1:2 or of 3:1 to 1:1.

In some embodiments, a CAR-t-NLP herein described can have a ratio of CAR to NLPs of 0.2:1 to 5:1.

Any measuring technique available in the art can be used to determine properties of the t-NLPs herein described. For example, techniques such as size exclusion chromatography (SEC), small angle X-ray scattering (SAXS), dynamic light scattering (DLS), nanoparticle tracking analysis, x-ray photoelectron microscopy, powder x-ray diffraction, scanning electron microscopy (SEM), transmission electron microscopy (TEM), cryo-electron microscopy (cryo-EM), and atomic force microscopy (AFM) can be used to determine average size and dispersity of the t-NLPs.

The inclusion of telodendrimers in the formulation was observed to improve both solubility and overall yield of the resulting immuno-NLP compositions, albeit modestly. Without being bound by theory, this enhancement is believed to arise, at least in part, from the intrinsic amphiphilic and stabilizing properties of the telodendrimer component, which may improve the solubility profile of telodendrimer-associated nanolipoprotein particles (T-NLPs) relative to traditional NLPs lacking the telodendrimer moiety. Telodendrimers, comprising a linear polyethylene glycol (PEG) backbone conjugated to dendritic clusters of hydrophobic moieties such as cholic acid or other lipid-interacting groups, are known to self-assemble into stable micellar or nanoparticulate structures in aqueous environments. When incorporated into NLPs during cell-free synthesis or post-assembly modification, these telodendrimer structures may enhance colloidal stability, reduce aggregation, and improve the compatibility of NLPs with hydrophobic or membrane-associated payloads such as intercellular communication and trafficking molecules (ICTMs), including CARs, cytokine receptors, and other membrane proteins.

In the present disclosure, the use of telodendrimer-containing formulations yielded a reproducible increase in the proportion of soluble NLP species, as well as in the recovery of functional ICTM: NLP complexes following synthesis and purification. These results suggest that T-NLPs can confer advantages in terms of manufacturability and formulation robustness, particularly for ICTMs that tend to aggregate, misfold, or partition inefficiently into conventional NLPs. Accordingly, telodendrimer components may be optionally included in any of the compositions or methods described herein to enhance solubility, facilitate protein incorporation, or improve particle homogeneity, without altering the fundamental structure or function of the ICTM payload as will be understood by a skilled person.

In some embodiments, CAR-NLPs herein described can further include additional lipids such as functionalized amphipathic compounds and/or one or more target proteins that can be added during the assembly of the NLP herein described.

The term “functionalized amphipathic compounds” in the sense of the disclosure indicates compounds having a hydrophobic portion and a hydrophilic portion in a configuration where the hydrophobic portion anchor is able to anchor the compound to the lipid bilayer of the NLP and the hydrophilic portion is presented on the NLP bilayer face following NLP assembly. In the functionalized amphipathic compounds in the sense of the disclosure, the hydrophilic portion of typically essentially consists of or comprises a hydrophilic functional group.

The term “functional group” as used herein indicates specific groups of atoms within a molecular structure that are responsible for a characteristic chemical reaction of that structure. Exemplary functional groups include hydrocarbons, groups containing double or triple bonds, groups containing halogen, groups containing oxygen, groups containing nitrogen and groups containing phosphorus and sulfur all identifiable by a skilled person.

The term “present” as used herein with reference to a compound or functional group indicates attachment performed to maintain the chemical reactivity of the compound or functional group as attached. Accordingly, a functional group presented on an amphipathic compound, is able to perform, under the appropriate conditions, the one or more chemical reactions that chemically characterize the functional group.

The use of functionalized amphipathic compounds enables attachment of various peptides or other biologics to the surfaces of the lipid of the NLP that allows some desired target features to be obtained, such as stability, affinity for a target molecule, and the like. Non-limiting examples of functional groups presented on functionalized lipids include chelated Ni atoms, azide, anhydride, alkynes, thiols, halogens, carboxy, amino, hydroxyl, and phosphate groups, and additional groups identifiable by a skilled person upon reading of the present disclosure.

In some embodiments, the functional group on the functionalized amphipathic compound can be a reactive chemical groups (e.g., azide, chelated nickel, alkyne, and additional reactive chemical groups identifiable by a skilled person), a biologically active compound (e.g., DNA, peptide, carbohydrate, and additional biologically active group identifiable by a skilled person), or a small molecule (e.g., cellular targeting compound, adjuvant, drug, and additional small molecules identifiable by a skilled person). In some embodiments, the functionalized amphipathic compound is a functionalized lipid compound. Functional groups that enhance the lipid solubility are referred to as hydrophobic or lipophilic functional groups. Functional groups that lack the ability to either ionize or form hydrogen bonds tend to impart a measure of lipid solubility to a drug molecule. The functional group can be attached to the lipid polar head through covalent or ionic bonds and “weak bonds” such as dipole-dipole interactions, the London dispersion force and hydrogen bonding, preferably covalent. Moreover, functionalization of the lipid can involve hydrophobic quantum dots embedded into the lipid bilayer. The following article is incorporated by reference in its entirety: R. A. Sperling, and W. J. Parak, “Surface modification, functionalization and bioconjugation of colloidal inorganic nanoparticles”, Phil. Trans. R. Soc. A 28 Mar. 2010 vol. 368 no. 1915 1333-1383 [50].

In some embodiments, functionalized amphipathic compounds can comprise one or more of 1,2-dipalmitoyl-sn-glycero-3-phosphoethanolamine-N-(6-((folate)amino) hexanoyl), 1,2-dipalmitoyl-sn-glycero-3-phosphoethanolamine-N-(6-azidohexanoyl), 1,2-dipalmitoyl-sn-glycero-3-phosphocthanolamine-N-(succinyl), 1,2-dioleoyl-sn-glycero-3-phosphoethanolamine-N-(succinyl), 1,2-dipalmitoyl-sn-glycero-3-phosphocthanolamine-N-(glutaryl), 1,2-diolcoyl-sn-glycero-3-phosphocthanolamine-N-(glutaryl), 1,2-diolcoyl-sn-glycero-3-phosphoethanolamine-N—(dodecanyl), 1,2-Dipalmitoyl-sn-Glycero-3-Phosphocthanolamine-N-(hexanoylamine), 1,2-dipalmitoyl-sn-glycero-3-phosphoethanolamine-N-(dodecanylamine), 1,2-Dipalmitoyl-sn-Glycero-3-Phosphothiocthanol, 1,2-diolcoyl-sn-glycero-3-phosphocthanolamine-N-[4-(p-malcimidomethyl)cyclohexane-carboxamide], 1,2-diolcoyl-sn-glycero-3-phosphoethanolamine-N-[4-(p-malcimidophenyl) butyramide], 1,2-diolcoyl-sn-glycero-3-phosphoethanolamine-N-[3-(2-pyridyldithio) propionate], 1,2-diolcoyl-sn-glycero-3-phosphoethanolamine-N-(biotinyl), 1,2-Diolcoyl-sn-Glycero-3-Phospho (Ethylene Glycol), 1,2-diolcoyl-sn-glycero-3-phosphocthanolamine-N-lactosyl, 1,2-distearoyl-sn-glycero-3-phosphoethanolamine-N-[dibenzocyclooctyl(polyethylene glycol)-2000], 1,2-distcaroyl-sn-glycero-3-1,2-distearoyl-sn-glycero-3-phosphoethanolamine-N-[succinyl(polyethylene glycol)-2000], 1,2-distcaroyl-sn-glycero-3-phosphocthanolamine-N-[carboxy (polyethylene glycol)-2000], 1,2-distcaroyl-sn-glycero-3-phosphocthanolamine-N-[malcimide (polyethylene glycol)-2000], 1,2-distearoyl-sn-glycero-3-phosphoethanolamine-N-[PDP(polyethylene glycol)-2000], 1,2-distcaroyl-sn-glycero-3-phosphoethanolamine-N-[amino (polyethylene glycol)-2000], glycol)-2000], 1,2-distearoyl-sn-glycero-3-phosphoethanolamine-N-[biotinyl(polyethylene glycol)-2000], phosphoethanolamine-N-[cyanur (polyethylene 1,2-distcaroyl-sn-glycero-3-glycol)-2000], cholesterol modified phosphoethanolamine-N-[folate (polyethylene oligonucleotides, cholesterol-PEG2000-azide, cholesterol-PEG2000-Dibenzocyclooctyl, cholesterol-PEG2000-malcimide, cholesterol-PEG2000-N-hydroxysuccinimide esters, cholesterol-PEG2000-thiol, cholesterol-azide, cholesterol-Dibenzocyclooctyl, cholesterol-malcimide, cholesterol-N-hydroxysuccinimide esters, cholesterol-thiol, C18 modified oligonucleotides, C18-PEG2000-azide, C18-PEG2000-Dibenzocyclooctyl, C18-PEG2000-malcimide, C18-PEG2000-N-hydroxysuccinimide esters, C18-PEG2000-thiol, C18-azide, C18-Dibenzocyclooctyl, C18-maleimide, C18-N-hydroxysuccinimide esters, C18-thiol. In some embodiments, CAR-NLPs can include additional signaling elements such as proteins found in immunological synapses (e.g. CD32), cytokines and interleukins, receptors for cytokines or interleukins.

In several embodiments herein described, Immuno-NLPs such as CAR-NLPs show different size, compositions, and homogeneity. Composition of a NLP can be detected by various techniques known in the art, such as high-performance liquid chromatography (HPLC), reverse phase high-performance liquid chromatography (RP-HPLC), mass spectrometry, thin layer chromatography, NMR spectroscopy and elemental analysis could be used to define the composition of the particles and additional techniques identifiable by a skilled person.

Size and compositions of the Immuno-NLPs such as CAR-NLPs can be characterized by SEC (size exclusion chromatography) traces which are used to separate out molecules in solution by their size and in some cases their molecular weights as will be understood by a skilled person.

In some embodiments, an Immuno-NLP such as CAR-NLP herein described can have a size ranging between 5 nm to 100 nm in diameter. In some embodiments, a CAR-NLP herein described can have a size ranging between 10 nm to 35 nm in diameter. In some embodiments, an Immuno-NLP such as CAR-NLP herein described can have a size ranging between 25 nm to 100 nm in diameter.

In preferred embodiments, an Immuno-NLP such as CAR-NLP herein described can have a size ranging between 5 nm to 100 nm in diameter with a ratio of telodendrimer to lipid is 1:10 to 1:1000, a ratio of scaffold protein to lipid of 1:30 to 1:100 and a ratio of telodendrimer to scaffold protein is 20:1 to 1:4.

More preferably among the most preferred embodiments, a CAR-NLP herein described can have a size ranging between 10 nm to 70 nm in diameter with a ratio of telodendrimer to lipid 1:50 to 1:500, a ratio of scaffold protein to lipid 1:30 to 1:100, and a ratio of MOMP to scaffold protein 5:1 to 1:2.

In most preferred embodiments, a CAR-NLP herein described has a size ranges between 25 nm to 50 nm in diameter. In the CAR-NLP, the ratio of telodendrimer to lipid is 1:100 to 1:200, the ratio of scaffold protein to lipid is 1:30 to 1:100, and the ratio of CAR to scaffold protein is 3:1 to 1:1.

In those embodiments, CAR-NLPs can solubilize a CAR with a solubility score≤ 20% of the total amount of the CAR protein in the mixture.

In particular, CAR-NLP with the above preferred and in particular, most preferred ratios are capable of increasing a CAR solubility from a solubility score of 10% to a solubility score greater than 70% when embedded in the resulting t-NLP-CAR particle, the percentage calculated with respect to the total amount of the CAR protein in the mixture.

In some of these embodiments, the increase in solubility allows CAR protein yield to be as high as 2 mg/mL cell-free reaction, and CAR insertion rate in the final construct to be as high as 50% or greater with respect to the total amount of CAR in the reaction mixture.

In particular, in some embodiments, Immuno-NLPs such as CAR-NLPs with the above preferred and in particular, most preferred ratios can provide an increase of 5-50% for the solubility of the transmembrane protein assembled into a t-NLP compared to the solubility of CAR in a mixture in absence of t-NLP. Once the material is purified, all of the subsequent material is present at 100% solubility.

Additionally, some embodiments of the Immuno-NLPs such as CAR-NLPs with the above ratios can allow oligomer CAR protein to be embedded in a single water-soluble nanoparticle, as well as the generation of 25 nm to 50 nm size nanoparticle suitable for in vivo application. Additionally, CAR-NLPs with the above ratios are particularly suitable in compositions, methods and systems directed to elicit an immunogenic response against in an individual.

In some embodiments, the Immuno-NLPs such as CAR-NLPs show a larger than expected size of approximately 40 nm than previously identified using other methods.

In some embodiments, any of the Immuno-NLPs such as CAR-NLPs herein described can be comprised in a composition together with a suitable vehicle. The term “vehicle” as used herein indicates any of various media acting usually as solvents, carriers, binders or diluents of an Immuno-NLP such as CAR-NLP comprised in the composition as an active ingredient.

In some embodiments, the composition of the disclosure comprises a same type of CAR-NLP or other Immuno-NLPs. In some embodiments, the composition can comprise more than one type of Immuno-NLPs such as one or more CAR-NLPs presenting different combination of CAR, other immune cell transmembrane protein and/or other components, and/or presenting a same or different combination of CAR and other immune cell transmembrane protein and/or other components at different ratios, as will be understood by a skilled person.

The term “lyophilization” (also known as freeze-drying or cryodesiccation) indicates a dehydration process typically used to preserve a perishable material or make the material more convenient for transport. Freeze-drying works by freezing the material and then reducing the surrounding pressure and adding enough heat to allow the frozen water in the material to sublime directly from the solid phase to gas.

If a freeze-dried substance is sealed to prevent the reabsorption of moisture, the substance may be stored at room temperature without refrigeration and be protected against spoilage for many years. Preservation is possible because the greatly reduced water content inhibits the action of microorganisms and enzymes that would normally spoil or degrade the substance.

Lyophilization can also cause less damage to the substance than other dehydration methods using higher temperatures. Freeze-drying does not usually cause shrinkage or toughening of the material being dried. In addition, flavors and smells generally remain unchanged, making the process popular for preserving food. However, water is not the only chemical capable of sublimation, and the loss of other volatile compounds such as acetic acid (vinegar) and alcohols can yield undesirable results.

Freeze-dried products can be rehydrated (reconstituted) much more quickly and casily because the process leaves microscopic pores. The pores are created by the ice crystals that sublimate, leaving gaps or pores in their place. This is especially important when it comes to pharmaceutical uses. Lyophilization can also be used to increase the shelf life of some pharmaceuticals for many years.

In pharmaceutical applications, freeze-drying is often used to increase the shelf life of products, such as vaccines and other injectables. By removing the water from the material and scaling the material in a vial, the material can be easily stored, shipped, and later reconstituted to its original form for injection.

In some embodiments, CAR-NLPs herein described can be used to perform protein transfer of the CAR protein onto membrane of immune cells (herein CAR-immune cells).

The term “immune cells” as used herein refers to cells that play a role in the body's defense system of an individual against substances identified by the body's defense system as foreign and potentially harmful. Immune cells are part of both the innate and adaptive immune systems, which work together to identify, neutralize, and destroy foreign invaders such as pathogens, such as bacteria, viruses, parasites, and fungi, and abnormal cells within the body. Immune cells are found in (and can be harvested from) all tissues of the body though they are most abundant in bone marrow, spleen, blood, and lymph tissues. Immune cells express a common set of cell surface proteins such as CD45 and depending on type a range of other cell surface marker proteins.

Accordingly, immune cells in the sense of the disclosure are cells that are involved in the immune system's response to damage or disease. The innate immune system recognizes a range of common molecular patterns that are associated with damage (i.e., damage associated molecular patterns) or pathogen invasion (i.e., pathogen associated molecular patterns). The adaptive immune system recognizes foreign or abnormal antigens, which is any substance that can trigger an immune response by being recognized as foreign by the immune system. Antigens can be molecules such as proteins, peptides, polysaccharides, lipids, or nucleic acids, and they are found on the surface of pathogens like bacteria, viruses, fungi, and parasites, as well as on abnormal cells such as cancer cells. They can also be non-microbial substances like pollen, egg white, and proteins from transplanted tissues and organs.

Antigens in the sense of this work are molecules that can be targeted by adaptive immune responses (comprising antibodies and reactive T cells). Antigens can originate from outside the body (exogenous antigens) or from within the body (endogenous antigens). In particular, exogenous antigens comprise antigens of bacteria, viruses, fungi, and parasites that invade the body and act as pathogens causing infections, toxins (which are harmful substances that can be enter the body or be produced for example by pathogens within the body), and allergens (which are substances like pollen, dust, and certain foods that can cause allergic reactions in some individuals). Endogenous antigens can encompass both abnormal antigens (which can be recognized and targeted by the immune system, and which when arising from mutations or DNA damage are often referred to as neoantigens), and autoantigens, or self-antigens, which are usually tolerated by the immune system but can become targets in autoimmune conditions.

The inventors have surprisingly found that chimeric antigen receptors (CARs) comprised in CAR-NLPs of the present disclosure can be transferred into a variety of immune cells in suspension without need of genetically engineering the immune cells, while resulting in a ≥2% of CAR-NLP treated immune cells with detectable and functional CAR on a cell membrane.

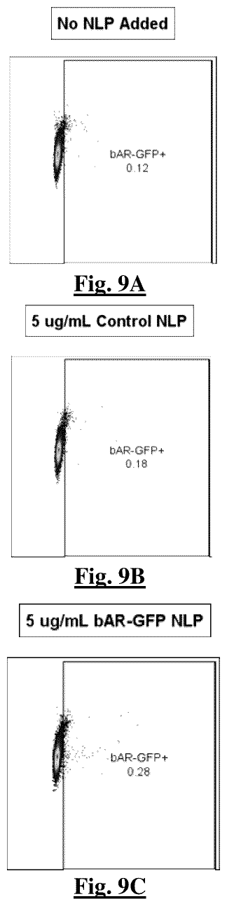

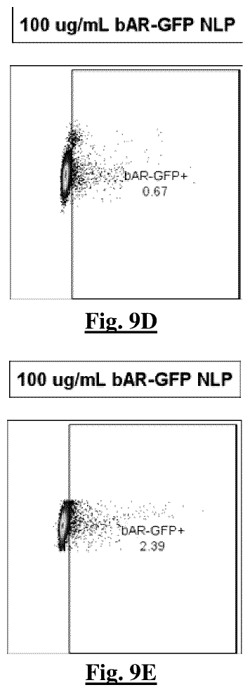

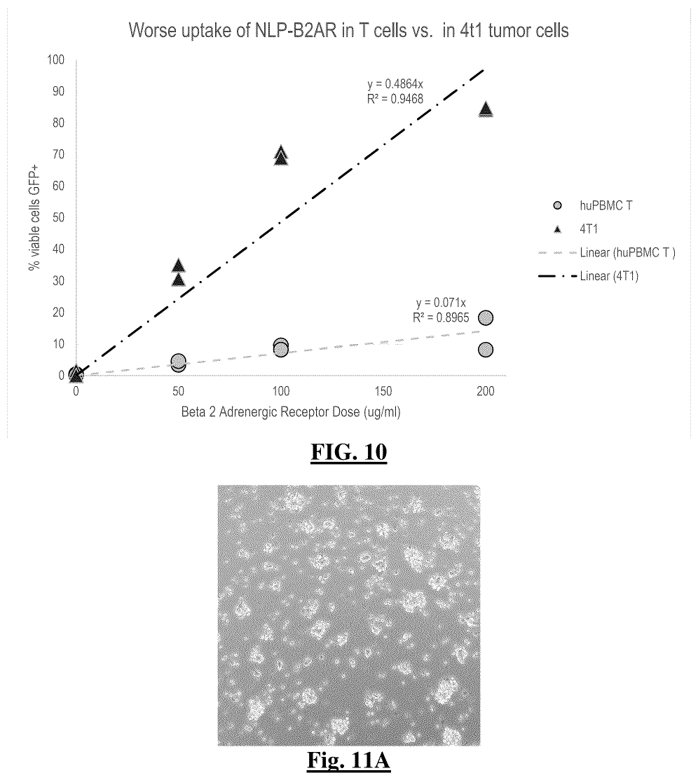

The inventors have found that previously published methods for NLP mediated cargo protein delivery, using beta 2 adrenergic receptor protein (B2AR)-NLPs as described in previous work [9], fails to deliver cargo protein into immune cells as indicated by similar numbers of GFP positive cells in GFP tagged B2AR-NLP treated and empty NLP and untreated control cells (see Example 5 and FIG. 9).

The inventors have also found that, uniquely for immune cells, treatment to induce cell activation increases NLP mediated transmembrane proteins exemplified by the B2AR protein delivery provided herein as representative examples of a transmembrane protein (see Example 5 and FIG. 9). No previous work known to the inventors has identified immune cell activation state as a result affective variable for NLP mediated protein uptake.

The inventors have further found that even with respect to immune cells treated with cell activation reagents, NLP mediated B2AR uptake using previously published methods is significantly less efficient (>6 fold less uptake) in immune cells compared to other cell types (see Example 5 and FIG. 10). Thus, high concentrations of cargo protein-NLP(at least 17 nanomolar cargo protein within NLPs or ˜100 nanomolar total NLP at 0.2CAR: nanodisc) is required to transfer proteins to immune cells. Higher NLP concentrations are known to induce toxicity in some cell types [51].

Theses minimum concentrations are expected to be effective for additional immune cell transmembrane proteins to be transferred into immune cells through NLP mediated proteins.

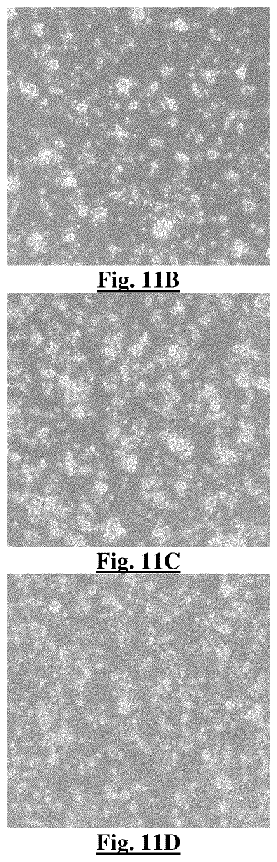

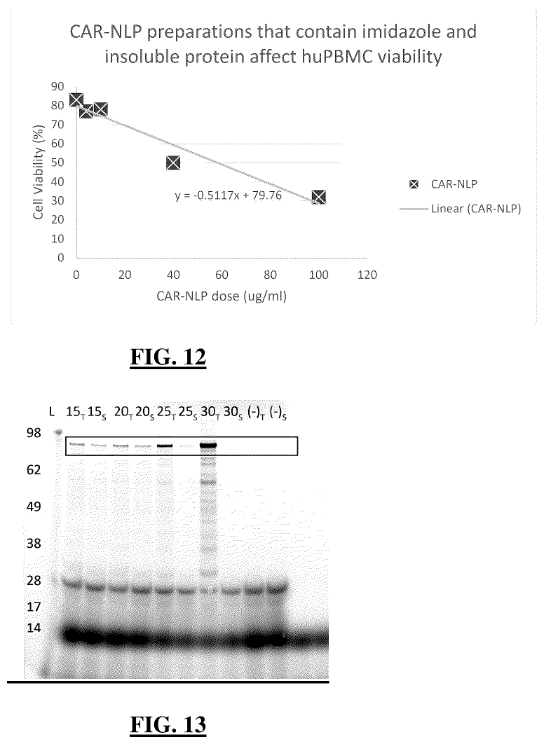

The inventors have also surprisingly found that using the cell free synthesis methods described herein [9, 40] to synthesize Immuno-NLPs and in particular CAR-NLP, and then using those methods to mediate protein transfer in immune cells, result in cell phenotype loss and cell death (see Example 6 and FIGS. 11A to 11D and FIG. 12). The inventors have identified two (2) sources of toxicity that previously have not been described for NLPs: insoluble protein and imidazole containing buffers.

The Immuno-NLP one-pot methods and Immuno-NLP two-pot methods have been designed by the inventors to ensure produce immuno-NLPs populations comprising a CAR protein at the concentrations required for transfer to immune cells while minimizing the amount of insoluble proteins reducing the related level in the NLP population to <10%. The Immuno-NLPs populations so obtained are expected to be effective for other immunoreceptors and immune cells transmembrane proteins as will be understood by a skilled person.

The inventors have additionally surprisingly found that the delivery can be performed on cells in suspensions while minimizing immune cells killing due to inherent toxicity of the treatment of the methods known to a skilled person for NLPs mediated protein transfer. Immuno-NLPs and in particular CAR-NLPs, can be used to decorate an immune cell surface with CAR protein [52].

Accordingly, in some embodiments, the Immuno-NLP particles and in particular CAR-NLP particle will be synthesized using cell free methods designed to reduce the level of insoluble protein to <10% and with careful removal of imidazole containing buffers or other equivalent buffers known or expected to be toxic to immune cells. The resulting Immuno-NLP treatment formulation comprises type and concentration of components that are non-toxic to immune cells, and thus maximize immune cells viability and functionality. In most preferred embodiments, the Immuno-NLP treatment formulation is a sterile formulation.

The inventors have also surprisingly found that in order for an effective immune transmembrane protein transfer into an immune cell, the immune cell requires a concurrent preceding activation of the immune cell.

Activation of immune cells in vitro involves isolating the cells, providing appropriate culture conditions, stimulating them with antigens or mitogens, providing necessary co-stimulatory signals, monitoring activation markers, and assessing functional responses as will be understood by a skilled person. In particular, a skilled person will understand that the activation of immune cells is an in vitro process that involves several well-established protocols and methodologies that aim to mimic the conditions under which these cells would be activated in vivo.

Accordingly, Immuno-NLP transfer methods of the disclosure comprise contacting the immune cells, with antigens to elicit specific immune responses, or with mitogens to perform non-specific stimulation for a time and under conditions to obtain an activated immune cell. This step can be performed with methods and techniques well known or identifiable by a skilled person.

For example, in some embodiments of the Immuno-NLP transfer methods of the disclosure, in order to perform activation, cells are cultured in appropriate conditions that include: a culture Medium such as RPMI-1640 or DMEM supplemented with fetal bovine serum (FBS), antibiotics, and other necessary growth factors maintained at 37° C. in a humidified atmosphere with 5% CO2. To activate immune cells, the immune cells are exposed to specific antigens for specific immune responses, such as peptide antigens presented by antigen-presenting cells (APCs) like dendritic cells, or mitogens such as non-specific stimulators like phytohemagglutinin (PHA), concanavalin A (ConA), or lipopolysaccharides (LPS) to induce a broad activation of immune cells. In some embodiments, activation can require additional co-stimulatory signals: such as CD28 on T cells interacting with B7 on APCs are crucial for full activation, cytokines like IL-2, IL-4, and IFN-γ to be added the culture to support the proliferation and differentiation of the activated cells [53].

The activation status of the immune cells can be monitored and/or detected by assessing the expression of specific surface markers such as CD69, CD25, and CD71 on T cells, indicating activation, e.g., by flow cytometry, cytokine Production such as IL-2, IL-6, TNF-α, and IFN-γ, e.g., by ELISA or intracellular cytokine staining.

In some embodiments, the activation status of the immune cells can be monitored and/or detected by performing functional assays to assess the functional capabilities of the activated immune cells: such as proliferation assays using thymidine incorporation or CFSE dilution assays along with cytotoxicity assays, which measure the ability of T cells or NK cells to kill target cells, assays like the chromium release assay, phagocytosis and ROS Production as will be understood by a skilled person.

Advances in vitro models can be used, such as 3D cultures to better mimic the tissue architecture and microenvironment, and microfluidic systems that replicate the complex interactions between different cell types and tissues, as will be understood by a skilled person [55].

In these embodiments, the Immuno-NLP transferred cell method for delivery of CAR or other immune cell transmembrane protein comprises of pre- or concurrently treating immune cells with activating reagents contacting the immune cells with an Immuno-NLP such as CAR nanolipoprotein particle for a time and under conditions resulting in the transfer of the cargo protein on the immune cells membrane. Cells suspended at between 0.1 and 10 million cells per ml should be dosed with from 17 nanomolar and 100nanomolar CAR concentration, preferably around 100 ug/ml CAR or other immune cell transmembrane protein in cell culture media at standard culture conditions for between one (1) hour and seven (7) days with six (6) to 24 hours preferred.

In other embodiments, Immuno-NLPs and in particular CAR-NLPs will be synthesized using methods for rapid decoration of NLPs incorporating modified lipids by mixing with tagged proteins to generate protein-NLP complexes [48, 49]. In these cases, the method for transmembrane protein delivery and in particular CAR delivery comprises pre- or concurrent treating immune cells with activating reagents and then contacting the immune cells with a protein-NLP complex at between 10 nanomolar and 5 micromolar cargo protein expandable to 10 nanomolar to 100 micromolar cargo protein at standard culture conditions for between six (6) hours and seven (7) days.

In certain embodiments, compositions comprising chimeric antigen receptor-nanolipoprotein particles (CAR-NLPs) are engineered to exhibit low cytotoxicity toward target immune cell populations, such as primary human T cells. Reduction of cytotoxic effects associated with CAR-NLP formulations has been found to depend on multiple interrelated factors, including the removal of purification reagents, the exclusion of improperly folded or aggregated proteins, and the optimization of the molar ratio between CAR protein and NLP scaffold components.

During protein purification, His-tagged CAR constructs are commonly isolated using immobilized metal affinity chromatography (IMAC), which employs imidazole as a competitive cluent to release the protein from the affinity resin. However, residual imidazole present in the final CAR-NLP formulation has been observed to negatively impact cell viability. Therefore, in preferred embodiments, the CAR protein is subjected to buffer exchange, dialysis, or other desalting techniques to substantially remove free imidazole prior to its incorporation into NLP complexes.

Insoluble or misfolded protein species, often present as aggregates or particulates following recombinant expression or purification, can also contribute to cytotoxic responses. These insoluble fractions are not efficiently integrated into NLP scaffolds and may instead associate non-specifically with cell membranes, triggering stress responses or apoptosis. Consequently, removal of insoluble protein components-such as through centrifugation, filtration, or preparative chromatography—is essential to ensure biocompatibility of the final formulation.

Finally, the molar ratio of CAR protein to NLP components must be carefully balanced to ensure that functional CAR moieties are displayed on the NLP surface at densities sufficient to mediate antigen-specific recognition, without overloading the scaffold or inducing unintended aggregation. Excess free CAR protein, or an overly dense packing of protein on the NLP surface, may contribute to physical instability or off-target interactions, both of which can elevate cytotoxicity. Thus, optimized formulations exhibit a controlled CAR: NLP ratio that maximizes functional receptor presentation while minimizing adverse cellular responses.

Collectively, these strategies-removal of purification components such as imidazole, exclusion of insoluble proteins, and control of CAR: NLP stoichiometry-synergistically reduce cytotoxicity and improve the safety profile of CAR-NLP constructs for use in vitro or in vivo.

In some embodiments, the methods and systems to transfer CAR protein with a CAR-NLP particle can be performed on immune cells involved in innate or adaptive cellular immunity, including monocytes/macrophages, natural killer cells, T cells and other phagocytic or lytic cell types.

In some embodiments, immune cell transmembrane proteins such as CAR proteins comprised in Immuno-NLPs of the disclosure can be transferred onto T-cells (T-lymphocytes), which are immune cells involved in cell-based immune response as will be understood by a skilled person. T-cells are involved in recognizing and responding to specific antigens presented by antigen-presenting cells (APCs) through their T-cell receptors (TCRs). This recognition triggers their activation, proliferation, and differentiation into effector cells that perform various immune functions, as will be understood by a skilled person upon reading of the present disclosure.

In particular, in some embodiments, CAR proteins or other immune cell transmembrane proteins comprised in Immuno-NLPs of the disclosure can be transferred onto helper T-cells (CD4+ T-cells), which are T-cells which coordinate other immune cells by releasing cytokines that enhance the immune response, activating B-cells to produce antibodies and for activating cytotoxic T-cells.

In some embodiments, CAR proteins or other immune cell transmembrane proteins comprised in Immuno-NLPs of the disclosure can be transferred onto Cytotoxic T-cells (CD8+ T-cells), which directly kill target cells which are for example infected or cancerous cells, by recognizing antigens presented by MHC class I molecules on the surface of the target cells as will be understood by a skilled person.

In some embodiments, CAR proteins or other immune cell transmembrane proteins comprised in Immuno-NLPs of the disclosure can be transferred onto NK/NK-T-cells: Natural Killer (NK) cells and NK T-cells can be engineered with CARs to enhance their innate ability to recognize and kill malignant-cells without prior sensitization, as will be understood by a skilled person [15].

In some embodiments, CAR proteins or other immune cell transmembrane proteins comprised in Immuno-NLPs of the disclosure can be transferred onto γδ T-cells, which are a subset of T-cells that possess a unique T-cell receptor (TCR) which can recognize antigens in a manner that is not restricted by the major histocompatibility complex (MHC) and can be transferred with a CAR protein to enhance their ability to target tumor cells directly as will be understood by a skilled person.

In some embodiments, CAR proteins or other immune cell transmembrane proteins comprised in Immuno-NLPs of the disclosure can be transferred onto monocytes or macrophages, which are a type of white blood cell that play a role in the immune system by engulfing and digesting cellular debris, foreign substances, microbes, and/or cancer cells. For example, macrophages can be engineered to express CARs, enabling them to target and phagocytose cancer cells. This approach can for example not only help in directly eliminating tumor cells but also assists in remodeling the tumor microenvironment, which is crucial for effective cancer therapy as will be understood by a skilled person.

In some embodiments, CAR proteins or other immune cell transmembrane proteins comprised in Immuno-NLPs of the disclosure can be transferred onto B-cells: a type of white blood cell that play a role in the adaptive immune system, specifically in humoral immunity. They are responsible for producing antibodies, which are proteins that can specifically bind to antigens (foreign substances) to neutralize or mark them for destruction by other immune cells. Engineering B-cells with CARs can turn B-cells into factories producing therapeutic antibodies. This approach leverages the natural role of B-cells in antibody production, potentially providing a continuous supply of therapeutic antibodies within the patient's body as will be understood by a skilled person [15].

In some embodiments, an Immuno-NLP transfer cell method of the present disclosure can be performed for transferring a cargo transmembrane protein contained within an Immuno-NLP into an immune cell, by treating the immune cells with pre- or concurrent stimulation of the immune cells with reagents to induce cell activation, wherein the Immuno-NLP has been formulated for use in immune cells by removal of imidazole buffers, then treated for sterility, for a time and under conditions resulting in the transfer of the cargo protein on the immune cells membrane, defined as culturing activated immune cells with cargo-nanolipoprotein particles with at least 10 nanomolar cargo protein concentration for between one (1) hour and 10 days with six (6) hours to 48 hours preferred.

In some embodiments, an Immuno-NLP transfer cell method of the present disclosure can be performed for transferring a CAR contained within an NLP into an immune cell, the method comprising: pre- or concurrently stimulated immune cells with reagents to induce cell activation, and contacting with a cargo-nanolipoprotein particle of the present disclosure, wherein the CAR-NLP has been formulated for use in immune cells by complete removal of insoluble protein (resulting in an undetectable amounts of insoluble protein) and buffer exchange to remove imidazole into phosphate buffered saline or similar physiological osmolarity aqueous buffer system, then treated for sterility, for a time and under conditions resulting in the transfer of the cargo protein on the immune cells membrane, defined as culturing activated immune cells with cargo-nanolipoprotein particles with at least 17.5 nanomolar cargo protein concentration for between one (1) hour and 10 days with six (6) hours to 48 hours preferred.

Immune cell populations obtained with the methods to perform an NLP mediated protein transfer of one or more CAR proteins and/or other immune cell transmembrane protein on the related membrane comprise the lipid-protein complex presented on the cell surface without the need to perform genetic engineering of the cells.

A transferred immune cell transmembrane protein, and in particular a CAR protein can be detected by either direct assays for CAR components or functional assays for CAR function identifiable by a skilled person.

In some embodiments, a detectable quantity of CAR or other immune cell transmembrane protein on the cell surface can be found in >2% of viable cells by flow cytometry for a fluorescent tag embedded in the CAR or other immune cell transmembrane protein, or by immunostaining for elements of the CAR, or by staining with a fluorescently tagged ligand of the antigen recognition domain.

In some embodiments, a detectable quantity of CAR or other immune cell transmembrane protein on the cell surface can be found where >2% of viable cells undergo detectable antigen specific activation in the presence of the ligand of the ABD of the CAR or other ligand in the extracellular portion of the immune cell transmembrane protein, as measured by flow cytometry for CD69, HLA-DR or by ELISPOT assay or similar marker of activation as understood by a skilled person.

In some embodiments, a detectable quantity of CAR or other immune cell transmembrane protein on the cell surface can be found by detecting a significantly increased target-cell killing for target cells expressing the ARD ligand compared to target cells that do not display the ligand.

In some embodiments, presence or absence of transgenes encoding CAR can be detected by methods for quantifying nucleic acids as will be understood by a skilled person, such as polymerase chain reaction (PCR), sequencing, and additional methods and techniques identifiable by a skilled person.

In some embodiments, detecting CAR or other immune cell transmembrane protein can be accomplished by either protein detection or functional assays. For example, protein detection can be accomplished via decorating the CAR with a fluorescent molecule prior to transfer to cells either through modification to include a fluorescent protein or conjugation with a small molecule fluorophore and detection by microscopy or fluorescent activated cell sorting, comparing CAR-NLP treated cells to untreated cells, or immunostaining cells with antibodies against elements of the CAR, e.g., the hinge domain, the scFv, relevant tag moieties, and analysis by microscopy or fluorescent activated cell sorting comparing CAR-NLP treated cells to untreated cells.

Exemplary functional assays include assays for detectable antigen specific activation in the presence of the ligand of the ARD of the CAR or corresponding portions in the extracellular region of another immune cell transmembrane protein as measured by flow cytometry for CD69, HLA-DR or by ELISPOT assay or similar marker of activation as understood by a skilled person, or where significantly increased target-cell killing is observed for target cells expressing the ARD ligand compared to target cells that do not display the ligand, or where significantly increased target-cell killing is observed for target cells expressing the ARD ligand compared to unmodified immune cells.

In some embodiments, a detectable quantity of CAR or other immune cell transmembrane protein on the cell surface can be found in >2% of viable immune cells by either flow cytometry using reagents specific for elements of the CAR, or flow cytometry using the ligand of the antigen recognition domain, or where >2% of viable cells undergo detectable antigen specific activation in the presence of the ligand of the ABD of the CAR as measured by flow cytometry for CD69, HLA-DR or by ELISPOT assay or similar marker of activation as understood by a skilled person, or where significantly increased target-cell killing is observed for target cells expressing the ARD ligand compared to target cells that do not display the ligand, or where significantly increased target-cell killing is observed for target cells expressing the ARD ligand compared to unmodified immune cells.

In certain embodiments, functional validation of the CAR-NLP compositions can be performed by assessing multiple biological outcomes in primary human T cells, including viability, surface expression of CAR protein, antigen binding capacity, and tumor cell killing activity. In some embodiments, even when uptake of CAR-NLPs by T cells is relatively limited—as indicated by low levels of CAR surface staining or CD19 antigen binding-significant tumor cytotoxicity can nevertheless be achieved based on findings that support the conclusion that sub-saturating levels of nanoparticle-delivered CAR protein are sufficient to confer functional antitumor activity (Example 6).

In some embodiments, simultaneous delivery of CAR-NLPs with cellular activation signals can improve the overall viability of treated T cells. Without wishing to be bound by theory, it is believed that concurrent activation facilitates membrane dynamics, protein trafficking, or receptor stabilization that can enhance the functional incorporation of CAR constructs while mitigating stress responses associated with partial or inefficient uptake.

Accordingly, in certain preferred embodiments, CAR-NLPs are co-administered with T cell activation stimuli (e.g., CD3/CD28 antibodies and/or cytokines) to support both efficient receptor presentation and maintenance of cell viability.

In some embodiments, CAR-NLPs can thus be used as a non-genomic platform for reprogramming immune effector cells, where even limited uptake can yield meaningful therapeutic responses, and co-treatment strategies can further optimize the cellular phenotype for therapeutic application as will be understood by a skilled person upon reading of the present application.

In some embodiments, a detectable quantity of CAR or other immune cell transmembrane protein on the cell surface can be found in >2% of viable immune cells (as assessed above) and the viability of the immune cells expressing the CAR retains >90% the viability of untreated immune cells, or >95% in preferred embodiments. Additionally in preferred embodiments, these cells are either simultaneously or pretreated with activating reagents such as anti CD3/CD28 reagents in the presence of IL2 or PMA or similar immune cell activating protocol, resulting in a population of actively replicating cells

In some embodiments, an Immuno-NLP, an Immuno-NLP population, and/or an Immuno-NLP engineered cell obtained with methods of the disclosure can be included in pharmaceutical compositions together with an excipient or diluent. In particular, in some embodiments, pharmaceutical compositions are described which contain Immuno-NLP engineered cell(s), in combination with one or more compatible and pharmaceutically acceptable vehicle, and in particular with pharmaceutically acceptable diluents or excipients.

The term “excipient” as used herein indicates an inactive substance used as a carrier for the active ingredients of a medication. Suitable excipients for the pharmaceutical compositions herein disclosed include any substance that enhances the ability of the body of an individual to intake Immuno-NLP, an Immuno-NLP population, and/or an Immuno-NLP engineered cell of the disclosure. Suitable excipients also include any substance that can be used to bulk up formulations with Immuno-NLP, an Immuno-NLP population, and/or an Immuno-NLP engineered cell of the disclosure to allow for convenient and accurate dosage. In addition to their use in the single-dosage quantity, excipients can be used in the manufacturing process to aid in the handling of Immuno-NLP, an Immuno-NLP population, and/or an Immuno-NLP engineered cell of the disclosure. Depending on the route of administration, and form of medication, different excipients can be used. Exemplary excipients comprise anti-adherents, binders, coatings disintegrants, fillers, flavors (such as sweeteners) and colors, glidants, lubricants, preservatives, sorbents and additional excipients identifiable by a skilled person.

The term “diluent” as used herein indicates a diluting agent which is issued to dilute or carry an active ingredient of a composition. Suitable diluents include any substance that can decrease the viscosity of a medicinal preparation.

In certain embodiments, compositions and, in particular, pharmaceutical compositions can be formulated for systemic administration, which includes parenteral administration and more particularly intravenous, intradermic, and intramuscular administration. In some embodiments, compositions and, in particular, pharmaceutical compositions can be formulated for non-parenteral administration and more particularly intranasal, intratracheal, vaginal, oral, and sublingual administration.

Exemplary compositions for parenteral administration comprise sterile aqueous solutions, injectable solutions, or suspensions including Immuno-NLP engineered cell(s). In some embodiments, a composition for parenteral administration can be prepared at the time of use by dissolving a powdered composition, previously prepared in a freeze-dried lyophilized form, in a biologically compatible aqueous liquid (distilled water, physiological solution or other aqueous solution).

Immuno-NLP, an Immuno-NLP population, and/or an Immuno-NLP engineered cell of the disclosure and/or related compositions can be used to target one or more antigens in vitro. Specifically, Immuno-NLP mediated engineering of immune cells can be used to artificially retarget immune cells to recognize antigens. As a result, Immuno-NLP engineered immune cells become more likely to adhere to antigen expressing cells or pathogens, or to develop cytotoxic responses, or to increase signaling in a desired manner.

Immuno-NLP, an Immuno-NLP population, and/or an Immuno-NLP engineered cell of the disclosure and/or related compositions can be used to target one or more antigens in an individual as will be understood by a skilled person.

The term “individual” as used herein in the context of treatment includes a single biological organism, having immune cells, such as animals and in particular higher animals and in particular vertebrates such as mammals and in particular human beings.

In some embodiments, Immuno-NLP engineered cell(s) or a composition herein described presenting one or more CAR proteins can also be administered to an individual alone or in combination with additional agents such as cytokines and small molecules to support the CAR-immune cell functionality. For example, long-acting polymer-coated IL-15 or recombinant human IL-7 can be co-administered to enhance CAR-T-cell proliferation and persistence [56].

In some embodiments, Immuno-NLP, an Immuno-NLP population, and/or an Immuno-NLP engineered cell of the disclosure can be used in methods for treating and/or preventing a condition in an individual.

The term “treatment” as used herein indicates any activity that is part of medical care for, or deals with, a condition, medically or surgically.

The term “prevention” as used herein indicates any activity which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” as used herein indicates a physical status of the body of an individual (as a whole or as one or more of its parts), that does not conform to a standard physical status associated with a state of complete physical, mental and social well-being for the individual. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

According to an eight aspect, a method and a system are described for treating or preventing a cancer condition, an infectious disease condition, an immune related condition, such as an autoimmune condition, graft versus host or host versus graft condition, a fibrotic condition, or conditions associated thereto in an individual, the method comprising administering to the individual one or more CAR immune cells of the present disclosure in an effective amount to elicit an immune response to CAR protein in the individual.

Timing and dosages of administration of CAR-immune cells alone or in combination with other agents to treat and/or prevent conditions herein described can vary depending on the individual treated, the effect to be achieved (treatment and/or prevention), and the severity of the infection as will be understood by a skilled person.

Suitable dosages can be used which provide the individual with a therapeutically effective amount or a prophylactically effective amount in accordance with the related embodiments of the disclosure. In particular, the term “effective amount” of one or more active ingredients refers to a nontoxic but sufficient amount of one or more drugs to provide the desired effect. For example, an “effective amount” of Immuno-NLP engineered cell(s) presenting a CAR protein associated with the treating and/or preventing (herein also “therapeutically effective amount” or “pharmaceutically effective amount”) a condition in the individual in which a target antigen possibly included in a target cell are present, refers to a non-toxic but sufficient amount of the Immuno-NLP engineered cell presenting the CAR protein to enable the target cell to take on desired functions in the treatment and/or prevention of such condition in the individual by promoting binding to the target antigen and/or the cell including the target antigen and resulting in effects on the target antigen expressing cell, for example killing the target antigen expressing cell as will be understood by a skilled person. A non-toxic amount of Immuno-NLP engineered cell(s) presenting the CAR protein can be identified by a person skilled in the art based on the guidelines and health reference levels provided by health organizations such as WHO and environmental protection agencies such EPA. This therapeutically effective amount of immuno-NLP is expected to be greater than the minimal dose to observe presence of the immuno-NLP within the treated immune cell population and is sufficient to result in effects on target antigen expressing cells in cell culture and the therapeutically effective amount of immuno-NLP treated immune cells is expected to be related to the cell dose required to alter disease behavior in animal models.

In certain embodiments, Immuno-NLP, an Immuno-NLP population, and/or an Immuno-NLP engineered cell of the disclosure can also be comprised in one or more systems to perform any one of the methods of the disclosure.

The Immuno-NLP one-pot system of the third aspect comprises one or more membrane forming lipids, an ICTM encoding polynucleotide, coding for the immunoreceptor protein, an SCP encoding polynucleotide coding for a scaffold protein and a cell-free reaction mixture for simultaneous combined or sequential use in the mixing of the Immuno-NLP one-pot method of the disclosure to provide an Immuno-NLP and the Immuno-NLP composition according to the second aspect of the present disclosure.

The Immuno-NLP two pot system to provide an Immuno-NLP according to the present disclosure, comprises one or more membrane forming lipids, an SCP polynucleotide coding for a scaffold protein, and a cell free reaction mixture for simultaneous combined or sequential use to provide a pre-assembled NLP in the two-pot method of the disclosure. The Immuno-NLP two-pot system of the disclosure further comprises an ICTM polynucleotide encoding for an immunoreceptor and a cell free reaction mixture for simultaneous combined or sequential use to provide an Immuno-NLP of the disclosure comprising the immunoreceptor attached to the membrane lipid discoidal bilayer of the assembled-NLP.

The Immuno-NLP engineered cell system comprises one or more Immuno-NLP transferred cells of the present disclosure and/or an Immuno-NLP transferred-cell population of the disclosure together with reagents for cell preparation and use in vitro or in vivo and/or for therapeutic administration. or delivery system, or necessary pre- or co-treatments in preparation for CAR-NLP immune cell therapy.

An Immuno-NLP one-pot system, an Immuno-NLP two-pot system, and an Immuno-NLP engineered cell system can comprise delivery system equipment, and cells for immune cell engineering, an NLP formulation and all other necessary hardware for NLP assembly, cell isolation and cell engineering, as will be understood by a skilled person.

An exemplary system to provide a CAR nanolipoprotein particle, can comprise one or more membrane forming lipids, a polynucleotide coding for a CAR protein and a polynucleotide coding for a scaffold protein for simultaneous combined or sequential use in the method to provide a NLP presenting a CAR, wherein the cell free lysate is derived from E. coli, yeast, insect or mammalian cells, with E. coli preferred, the plasmid: cell free reaction mix ratio is between 0.1 and 100 mg/ml with 10⁻³⁰ mg/ml preferred, the ratio of polynucleotides encoding CAR and scaffold protein is between 2:1 and 600:1 with 20:1 to 60:1 preferred, and the lipid: cell free mix ratio is between 0.1 mg/ml and 100 mg/ml with 2-20 mg/ml preferred, and where the synthesized CAR-NLP is co-purified.

The systems herein disclosed can be provided in the form of kits of parts. In a kit of parts for the production of Immuno-NLPs herein described, the immune cell transmembrane protein such as CAR and the scaffold protein can be included in the kit as a protein alone or in the presence of lipids/detergents for transition into nano-particles. The CAR and/or the scaffold protein can be included as a plasmid or PCR DNA product for transcription/translation. The indicator protein may be included as encoded RNA for translation.

In a kit of parts for delivering CAR-immune cell(s) to a target antigen and/or cell in vitro and/or to an individual in vivo, the CAR-immune cell(s) can be comprised together of cells, CAR-NLPs, excipients and additional components identifiable by a skilled person.

In a kit of parts, a polynucleotide, amphipathic lipid, target protein and/or scaffold protein, CAR-NLPs, and additional reagents are comprised in the kit independently possibly included in a composition together with suitable vehicle carrier or auxiliary agents. For example, a polynucleotide can be included in one or more compositions alone and/or included in a suitable vector, and each polynucleotide in a composition together with a suitable vehicle carrier or auxiliary agent. Furthermore, the target protein can be included in various forms suitable for appropriate incorporation into the NPL.

Additional components can include labeled polynucleotides, labeled antibodies, labels, microfluidic chip, reference standards, and additional components identifiable by a skilled person upon reading of the present disclosure. In particular, the components of the kit can be provided with suitable instructions and other necessary reagents in order to perform the methods here disclosed. The kit will normally contain the compositions in separate containers. Instructions, for example written or audio instructions, on paper or electronic support such as tapes or CD-ROMs, for carrying out the assay, may be included in the kit. The kit can also contain, depending on the particular method used, other packaged reagents and materials (e.g., wash buffers and the like).

The terms “label” and “labeled molecule” as used herein refer to a molecule capable of detection, including but not limited to, radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens), and the like. The term “fluorophore” refers to a substance or a portion thereof which is capable of exhibiting fluorescence. As a consequence, the wording “labeling signal” as used herein indicates the signal emitted from the label that allows detection of the label, including but not limited to radioactivity, fluorescence, chemoluminescence, production of a compound in outcome of an enzymatic reaction and the like.

Further details concerning the identification of the suitable carrier agent or auxiliary agent of the compositions, and generally manufacturing and packaging of the kit, can be identified by the person skilled in the art upon reading of the present disclosure.

### EXAMPLES

The NLPs, cells, compositions methods and system herein disclosed are further illustrated in the following examples, which are provided by way of illustration and are not intended to be limiting.

In particular, the following examples relate to specific CAR structures. NLP compositions and immune cells which are provided herein to show a proof of principle in the us of the CAR-NLPs of the disclosure and related cells compositions methods and systems. A skilled person will be able to adapt the indications of the present examples to additional CAR proteins, NLPs, and immune cells.

The following materials and methods were used.

### Example 1. Exemplary Structures of Immune Cell Transmembrane Proteins

Transmembrane proteins are membrane proteins that are involved in various cellular processes, including signaling, transport, and cell adhesion. Structurally and for the purpose of the present disclosure, transmembrane proteins can be divided into three main domains: the extracellular domain, the transmembrane domain, and the intracellular domain as shown in the schematic representation of FIG. 1A.

When the transmembrane protein is attached to a membrane cell, the extracellular domain of transmembrane proteins is located outside the cell and interacts with the external environment. The structure of the extracellular domain can vary significantly among different transmembrane proteins, but it often includes multiple subdomains that contribute to its specific function(s). For instance, in type-I transmembrane proteins, the extracellular segment is typically involved in cell signaling and adhesion processes, and it can undergo conformational changes to regulate these functions.

In some transmembrane proteins herein identified as immunoreceptors, the extracellular domain contains binding sites for ligands, which can include hormones, neurotransmitters, or other signaling molecules, as shown in the schematic of FIG. 1B. In particular, in immunoreceptors, the extracellular domain comprise a ligand binding domain which is configured to specifically bind a corresponding ligand (LRD) as will be understood by a skilled person.

The transmembrane domain is the portion of the transmembrane protein that spans the lipid bilayer of the cell membrane. This domain is predominantly composed of hydrophobic amino acids, which interact with the hydrophobic core of the lipid bilayer. The transmembrane domain can consist of one or multiple alpha-helices or beta-barrels. Alpha-helical transmembrane segments are more common and are found in the inner membranes of bacterial cells and the plasma membranes of eukaryotic cells. The transmembrane domain not only anchors the protein in the membrane but also plays a role in the conformational changes required for the protein's function as will be understood by a skilled person (see FIGS. 1A and 1B).

The intracellular domain of transmembrane proteins is located inside the cell and interacts with the cytoplasmic environment. This domain often contains sites for interactions with intracellular signaling molecules, cytoskeletal elements, or other proteins. The intracellular domain can also be involved in the regulation of the protein's activity and in transmitting signals from the extracellular environment to the cell's interior. For example, in many receptors, the binding of a ligand to the extracellular domain induces a conformational change that is transmitted through the transmembrane domain to the intracellular domain, initiating a cascade of intracellular signaling events (see FIGS. 1A and 1B).

A representative example of the structure of transmembrane protein which can be found on immune cells and which are also immunoreceptors is provided by G protein-coupled receptors (GPCRs), in which the extracellular domain binds to ligands, triggering a conformational change that activates intracellular signaling pathways as will be understood by a skilled person.

The structure of transmembrane proteins schematically shown in FIGS. 1A and 1B is mimicked by engineered constructs which are used in engineering of immune cells such as the structure of CAR proteins schematically illustrated in FIG. 1C. The illustration of FIG. 1C schematically shows that in CAR proteins, the Ligand Recognition Domain (LRD) is an Antigen Recognition Domain (ARD), which is linked to the Transmembrane Domain through a hinge linker, which is in turn linked to an intracellular domain formed by signaling domains derived from immune cell proteins.

An additional discussion of the structures of CAR proteins is provided in the following Example 2.

### Example 2. Exemplary CAR Structures

Exemplary CAR protein structures are reported in the schematic illustrations of FIGS. 1C and 2A to 2E.

FIG. 1C schematically shows different regions of a CAR protein in the sense of the disclosure and possible structure that can alternatively occupy those regions.

For example, in the illustration of FIG. 1C, the Antigen Recognition Domain of the CAR protein is shown to be possibly a CD28 extracellular domain with its single Ig variable-like domain linked to a GCSF N-terminal immunoglobulin-like domain that is involved in ligand binding and receptor dimerization. In the alternative, the illustration of FIG. 1C shows an antigen recognition domain or an scFv CD19 consisting of two variable domains (VH and VL) from an antibody, linked together by a linker.

The schematic of FIG. 1C also shows exemplary hinge regions formed by CD8, CD24, CD28 or IgG. These domains can vary in terms of length and flexibility, which alters ability of the ARD to bind.

The schematics of FIG. 1A further show a Transmembrane Domain of the CAR, which can be possibly formed by regions from CD8, CD28 or CD3.

The schematics of FIG. 1 also show exemplary intracellular Signaling Domains selected from CD27, CD28, 41BB, OX40.

The schematics of FIG. 1 additionally show exemplary costimulatory Domains selected from CD3ζ.

The schematics of FIG. 2 show differences in CAR design by generation.

1^{st} generation CAR (FIG. 2A) contain an ABD, transmembrane domain and costimulatory domain from CD35. Generation 1 CAR-T cells showed limited efficacy and

durability in clinical trials [57]. The techniques described in this work are known to work with 1 ^{st} generation CAR constructs.

2^{nd} generation CAR constructs (FIG. 2B) add a costimulatory domain (such as CD28 or 41BB signaling domain), which increases cell persistence and overall efficacy. The techniques described in this work are known to work with 2^{st} generation CAR constructs.

3^{rd} generation CAR constructs (FIG. 2C) combine multiple costimulatory domains. The techniques described in this work are expected to work with 3^{rd} generation CAR constructs.

4^{th} generation CAR constructs, also referred to as TRUCKs (FIG. 2D), combine 2^{nd} or 3^{rd} generation CAR constructs with transgenes encoding for cytokines to increase cell durability. The techniques described in this work are expected to work with 4^{th} generation CAR constructs, such as those schematically illustrated in FIG. 2E.

Newer generations of CAR constructs ([58] not shown) can include incorporation of additional/novel costimulatory domains (which the techniques in this work are expected to work for), or additional signaling elements such as self-destruction features or combinatorial sensing to limit activation to within tumor microenvironments, and/or gene editing to reduce exhaustion.

### Example 3. Exemplary Telodendrimers Suitable to be Included in Immuno-NLP

Telodendrimers are a class of hybrid polymers that combine linear polymer segments with dendritic (branched) structures. These unique architectures allow for multifunctional capabilities, making them highly versatile for various biomedical applications, particularly in drug delivery.

The structure of telodendrimers typically consists of a linear polyethylene glycol (PEG) segment and a dendritic segment. The dendritic segment can be hydrophobic or amphiphilic, often terminating in functional groups such as cholic acid or hydrophobic drugs.

Telodendrimers can self-assemble into micelles or other nanostructures in aqueous solutions, driven by the hydrophobic interactions of their dendritic segments.

Telodendrimers can be functionalized with various molecules, including drugs, proteins, and targeting agents, enhancing their utility in therapeutic delivery and diagnostic applications.

For example, Cys-telodendrimers are a specialized type of telodendrimer that incorporate cysteine (Cys) residues into their structure. These cysteine moieties play a crucial role in forming reversible disulfide bonds, which can enhance the stability and functionality of the telodendrimer-based nanocarriers.

FIG. 3A shows a schematic representation of exemplary Cys-telodendrimer suitable to be included in CAR-t-NLPs herein described.

The exemplary illustration of FIG. 3A shows that Cys-telodendrimers include cysteine residues that enable the formation of disulfide bonds. This feature is particularly useful for creating cross-linked micelles.

The exemplary illustration of FIG. 3A also shows that similar to other telodendrimers, Cys-telodendrimers often feature a polyethylene glycol (PEG) segment that provides hydrophilicity and biocompatibility.

The exemplary illustration of FIG. 3A further shows that the dendritic part of the telodendrimer can be functionalized with various moieties, such as cholic acid, to enhance drug loading and delivery capabilities.

Cys-telodendrimers help stabilize the phospholipid bilayers in nanodiscs. The cysteine residues enable the formation of disulfide bonds, which can cross-link the telodendrimers, providing additional structural stability to the nanodiscs. This stabilization is crucial for maintaining the integrity of the nanodiscs under physiological conditions and during storage.

The integration of telodendrimers, including those with cysteine residues, has been shown to reduce the polydispersity of nanodiscs. This means that the nanodiscs are more uniform in size, which is beneficial for consistent drug delivery and diagnostic applications. The reduced polydispersity is achieved through the controlled self-assembly facilitated by the telodendrimers.

The presence of telodendrimers in the nanodisc formulation can also improve the solubility and yield of the nanoparticles. This is particularly important for the production of nanodiscs in a cell-free system, where the addition of telodendrimers has been shown to increase the overall yield and solubility of the produced nanoparticles.

Similar considerations apply to the exemplary telodendrimer functionalized with histidine (His-telodendrimers). His-telodendrimers are a specialized type of telodendrimers that incorporate histidine (His) residues into their structure.

FIG. 3B shows a schematic representation of exemplary His-telodendrimer suitable to be included in CAR-t-NLPs herein described.

The exemplary illustration of FIG. 3B shows that His-telodendrimers include histidine residues, which can impart pH-sensitive properties. Histidine has an imidazole side chain with a pKa around 6.0, making it responsive to slight changes in pH.

The exemplary illustration of FIG. 3B also shows that like other telodendrimers, His-telodendrimers typically feature a polyethylene glycol (PEG) segment that provides hydrophilicity and biocompatibility.

The exemplary illustration of FIG. 3B further shows that the dendritic part of the telodendrimer can be functionalized with various hydrophobic or amphiphilic moieties, enhancing drug loading and delivery capabilities

His-telodendrimers can form reversible disulfide bonds, which help stabilize the phospholipid bilayer of nanodiscs. This stabilization is crucial for maintaining the integrity of the nanodiscs under physiological conditions and during storage.

The incorporation of His-telodendrimers enhances the overall stability of nanodiscs, reducing the likelihood of aggregation and improving their circulation time in the bloodstream.

His-telodendrimers provide convenient covalent attachment sites for functional groups, such as targeting ligands and imaging agents. This allows for the customization of nanodiscs for specific therapeutic and diagnostic applications.

Functionalization with targeting ligands can enhance the specificity of nanodiscs for particular cells or tissues, such as cancer cells, improving the precision of drug delivery.

The addition of His-telodendrimers to the nanodisc formulation can increase the solubility and yield of the nanoparticles. This is particularly beneficial for the production of nanodiscs in a cell-free system, where the presence of His-telodendrimers has been shown to enhance the overall yield and solubility of the produced nanoparticles.

Additional properties of cys-and his-telodendrimers will be identified by a skilled person. Additional functionalization of telodendrimers that can be used in connection with Immuno-NLPs of the disclosure will also be identified by a skilled person upon reading of the present disclosure.

### Example 4. Exemplary Apolipoprotein Fragment Suitable to be Used with Immuno-NLPs

Various scaffold proteins cam be used in connection with Immuno-NLPs of the disclosure. In particular, scaffold proteins that can be used are apolipoproteins and related fragments as will be understood by a skilled person.

Exemplary apolipoprotein fragments that can be used in connection with Immuno-NLPs are shown in FIG. 4A to FIG. 7.

In particular, FIG. 4A shows a Codon optimized Δ49ApoA1 derived through Genscript optimization. ApoA1 is the main protein component of HDL and plays a crucial role in lipid metabolism and cholesterol transport. In the context of nanodiscs, ApoA1 or its engineered variants wrap around the lipid bilayer, stabilizing the structure and maintaining its discoidal shape.

Mouse Δ49ApoA1 is a truncated version of ApoA1, where the first 49 amino acids are deleted. This truncated form retains the ability to stabilize nanodiscs but may exhibit different properties compared to the full-length protein. The deletion might affect the protein's interaction with lipids and its overall structural stability.

The truncated form of Δ49ApoA1 can provide enhanced stability to the nanodiscs, making them more suitable for various experimental conditions. The deletion of the N-terminal region in Δ49ApoA1 can simplify the assembly process of nanodiscs, potentially leading to more uniform and monodisperse particles. Additionally, Δ49ApoA1 nanodiscs can be used in a wide range of applications, from structural biology to vaccine development, due to their ability to mimic natural lipoprotein particles effectively.

FIG. 4B shows a Codon optimized ApoE4, 22k derived through Genscript optimization. Apolipoprotein E (ApoE) is a critical protein involved in lipid metabolism and is essential for the transport and clearance of cholesterol and other lipids in the body. There are three major isoforms of human ApoE: ApoE2, ApoE3, and ApoE4.

Mouse ApoE4, 22k is a specific variant of the apolipoprotein E4 (ApoE4) protein, which is a truncated form of the full-length ApoE4. The truncated form of ApoE4 lacks cysteine residues, simplifying recombinant protein expression and purification, which enhances the efficiency of nanodisc assembly. Additionally, ApoE4, 22k nanodiscs can be used in a variety of applications, from structural biology to therapeutic delivery systems, due to their ability to form stable, monodisperse particles.

ApoE4, 22k nanodiscs are known to be versatile as they provide a native-like lipid environment, making them ideal for studying membrane proteins and lipid interactions in conditions that closely mimic physiological states.

Apolipoproteins and related fragments can be codon optimized for expression in a specific system as will be understood by a skilled person.

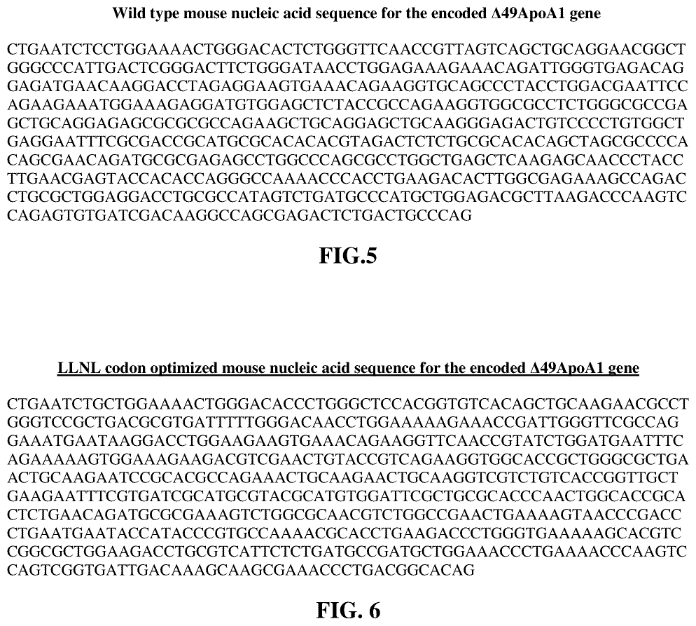

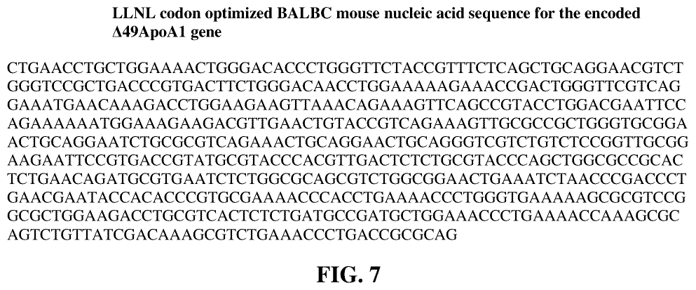

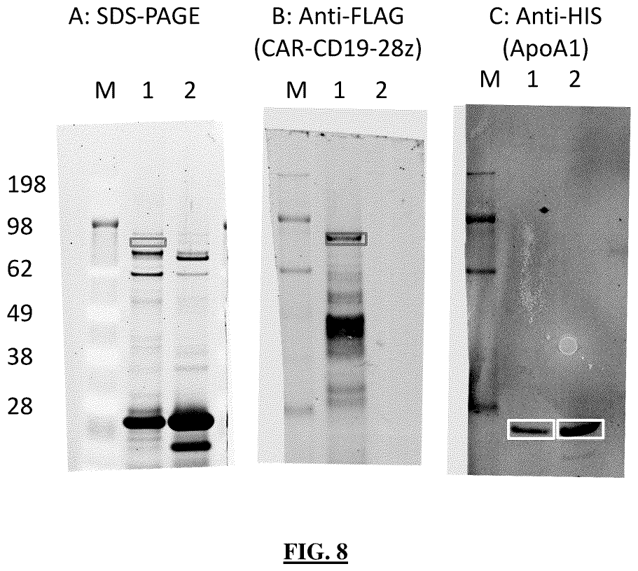

FIGS. 5 to 7 show exemplary optimizations of Δ49ApoA, in mouse (FIG. 6) and BALBC mouse (FIG. 7), in comparison with the wild type mouse nucleic acid sequence for the encoded Δ49ApoA1 gene (FIG. 5).

Additional codon optimization for Δ49ApoA or other apolipoproteins or scaffold proteins and related fragments can be identified by a skilled person.

### Example 5. Synthesis of Soluble CAR-NLP and Other Cargo-NLPs Is Improved At Low Temperature

Beta2 adrenergic receptor-delta70 and ApoA1 were co-expressed at 15:1 ratio in 1 ml of Biotech rabbit E. Coli cell free lysate in a reaction chamber with the addition of 2 mg/ml DMPC at either 20° C. of 30° C. for either six (6), 18, 24, or 48 hours. NLPs were purified from the reaction mix using nickel affinity resins and eluted with imidazole buffer, then fractions with high concentrations were pooled. The NLPs suspension was then dialyzed against PBS. Insoluble material was removed by centrifugation at 14,000g, then total and soluble fractions were run on PAGE to analyze protein density for ApoA1 and beta-2 adrenergic receptor.

Synthesis at 30° C. resulted in significantly higher total protein concentration, however the solubility was poor (8-20%). While synthesis at 20° C. resulted in much less total protein, the increased solubility (70-80%) resulted in a higher soluble protein concentration than at higher temperature.

Cargo protein loading into NLPs followed the same trend as total protein yield: 30° C. synthesis resulted in an overall higher cargo protein: NLP ratio, however the cargo protein: NLP ratio in the soluble fraction was significantly (>2×) higher.

ApoA1 and CAR were co-expressed at a 20:1 plasmid ratio in Iml Biotech rabbit E. Coli cell-free lysates in a reaction chamber with the addition of 2 mg/ml lipids, at 15° C., 20° C., 25° C. or 30° C. for 18 hours. Plasmids: 20 μg CD19 CAR-28Z (Addgene Plasmid #135991 cloned into pIVEX2.3 vector) and I μg ApoA1 (described in [9]). 100 μL of 20 μg/ml DMPC was added. NLPs were purified from the reaction mix using nickel affinity resins and eluted with imidazole buffer, then fractions with high concentrations were pooled. The NLPs suspension was then dialyzed against PBS. Insoluble material was removed by centrifugation at 14,000g, then total and soluble fractions were run on PAGE to analyze protein density.

Increasing temperature increases total protein concentration, but drastically reduces soluble protein concentration, and reduces CAR: NLP ratio for both CAR and for beta2 adrenergic receptor.

Ability to bind the ligand specified by the receptor was demonstrated by blotting CD19 containing cell lysates onto a membrane then incubating with CAR-nanodisc suspensions, then washing and detecting CD19-CAR binding by antibodies against the CAR.

### Example 6. Delivery In T-cells without Cytotoxicity Requires Removal of Insoluble

### Material and Imidazole Buffers

Efficient uptake of membrane protein into T-cells requires T-cell activation (not required for adherent cell lines), and requires higher concentrations of NLP for equivalent engineered NLPs containing GFP tagged beta2 adrenergic receptors (B2AR) or empty NLPs comprised of lipid and apolipoprotein, as prepared in Example 5 [9].

Human peripheral blood T cells (>90% CD3+) isolated from donor blood by negative selection (PB03NC-1, Charles River) were thawed to resting culture overnight in tecsmacs media (130-097-196, Miltenyi Biotech), then treated for 24 hours with empty NLPs at 5 μg/ml, B2AR-NLPs at 5 μg/ml [9], and B2AR-NLPs at 100 μg/ml. For activation, the T cells were treated with 5 μg/ml B2AR-NLP in the presence of 1:50 anti-CD3/CD28 reagents (Transact, 130-111-160 Miltenyi) and 40 IU/ml 112 (PCH0021, Gibco). As controls, untreated T cells were maintained in media with no additives. Cells were then collected, washed, stained with PI for viability and the GFP+ fraction of viable cells was analyzed.

The results shown in FIGS. 9A to 9E show no evidence of B2AR uptake in T-cells treated with either 5 or 100 μg/ml B2AR-NLP after one (1) day, as indicated by similar numbers of GFP+ cells in untreated, empty NLP and B2AR-NLP treated cells. The results, as reported in FIG. 9E, show that uniquely for T-cells, pre- or co-treatment with activating reagents, such as anti-CD3/CD28 reagents+IL2, was necessary for NLP mediated membrane protein delivery to T-cells.

Thus, surprisingly, the methods described herein [9, 41, 43, 59] do not deliver membrane proteins to T-cells, in direct contrast to claims in previous papers that NLP mediated membrane protein delivery works in all or most cell types. Using the B2AR-NLPs developed in previous studies we observe, surprisingly, that uptake in T cells is not detectable, even when dosed with 20-fold more material than used previously.

Surprisingly, activation using anti-CD3/CD28 reagents improves uptake in T cells.

The results in FIG. 10 compare dose-response uptake for activated human peripheral blood T cells and an adherent tumor cell line (4t1).

Both cell types were cultured according to manufacturer's specifications: T cells (PB03NC-1, Charles River) were thawed to resting culture overnight in tecsmacs media (130-097-196, Miltenyi Biotech) and then were treated with 1:50 anti-CD3/CD28 reagents (Transact, 130-111-160 Miltenyi) and 40 IU/ml 112 (PCH0021, Gibco), and 4t1 (CRL-2539, ATCC) were cultured in RPMI-1640+10% serum, then both were dosed with between 12.5 and 200 μg/ml GFP tagged B2AR-NLP for 24 hours. After 24 hours, 4tl were trypsinized, then both lines were washed 3×with PBS+1% BSA, and then stained with PI and analyzed by flow cytometry to determine GFP+ fraction of viable cells.

While detectable uptake can be found in both cell lines, a 6-fold greater uptake per dose was observed in 4tl cells compared to huPBMC T cells. Part of this difference may arise from the significant size difference between these cell types: T cells have an average diameter of 8-10 μm compared to ˜30-40 μm for 4t1, which corresponds to a ˜5-fold difference in surface area.

Significantly higher concentrations of cargo protein-NLP must be used in activated T cells compared to other cell types. For example, achieving >50% cargo protein+ fraction would require doses of 6 micromolar, or 700 μg/ml,. In contrast, previous studies used 100 nM (10 μg/ml) [9, 59] to 500 nM. Because of this higher dosing concentration, there is a higher risk of toxicity. Previous studies have demonstrated that empty-vector NLPs can be toxic at high concentrations.

The NLPs suspension for membrane protein delivery to T-cell needs to be formulated differently than in previous work because of both the greater sensitivity of T-cells and the 10⁻¹⁰⁰×greater concentrations of nanodiscs required for delivery.

CAR-NLPs, when expressed using the cell free methods described herein, [9] are poorly soluble, defined as <20% of CAR protein remaining in the supernatant when centrifuging the total cell-free mixtures following completion of the cell-free reaction (e.g., by a microcentrifuge at max speed for 10 minutes). Previous work has not noted that NLP suspensions containing an insoluble protein fraction affect cell viability [9].

Surprisingly, the inventors find that treatment of T-cells with chimeric antigen receptor suspensions with insoluble protein at high concentrations (40 or 100 μg/ml vs. 10) resulted in cell death (as indicated by lack of T-cell clustering) as shown in FIG. 10. HuPBMC T cells (PB03NC-1, Charles River) were thawed to resting culture overnight in Tecsmacs media (130-097-196, Miltenyi Biotech), and then were treated with 1:50 anti-CD3/CD28 reagents (Transact, 130-111-160 Miltenyi) and 40 IU/ml 112 (PCH0021, Gibco) and between 0 and 100 μg/ml, the CAR-NLP were synthesized [9] for one (1) day. At concentrations of ≤10 μg/ml, T cell activation can be seen as indicated by formation of T cell clusters. At 40 or 100 μg/ml, cluster formation is absent, indicating loss of T cell function.

Imidazole containing buffers are used in nickel purification steps and are known to promote protein solubility and stability. Previous work generated nanodisc suspensions in imidazole and directly diluted this suspension into cell culture media when treating cell cultures. The results reported in FIGS. 11A to 11D show that addition of nanodisc membrane proteins in dilute imidazole buffer to T-cell cultures affected viability significantly.

CAR-NLP were synthesized at 20° C. for 18 hours in cell free reaction mixes by mixing CAR and scaffold protein polynucleotides at 30:1 in the presence of 2 mg/ml DMPC, using nickel resins and imidazole elution buffers, centrifuged at 14 kg to remove insoluble material, and then filter sterilized through a 0.2 μm PES syringe. HuPBMC T cells (PB03NC-1, Charles River) were thawed to resting culture overnight in Tecsmacs media (130-097-196, Miltenyi Biotech), then were treated with 1:50 anti-CD3/CD28 reagents (Transact, 130-111-160 Miltenyi) and 40 IU/ml 112 (PCH0021, Gibco), and between 0 and 100 μg/ml CAR-NLP for one (1) day, followed by washing three (3) times with PBS+1% BSA, staining with PI and FACS to determine viable cell fraction (FIGS. 11A to 11D). Significant viability loss is observed at 40-100 μg/ml doses (see FIG. 12).

In contrast, FIG. 13 shows that removal of imidazole buffer reduces CAR-NLP cytotoxicity. CAR-NLP were synthesized at 20° C. for 18 hours in cell free reaction mixes by mixing CAR and scaffold protein polynucleotides at 30:1 in the presence of 2 mg/ml DMPC, using nickel resins, dialyzed into PBS, centrifuged at 14 kg to remove insoluble material, and then filter sterilized through a 0.2 μm PES syringe filter. HuPBMC T cells (PB03NC-1, Charles River) were thawed to resting culture overnight in Tecsmacs media (130-097-196, Miltenyi Biotech), and then were treated with 1:50 anti-CD3/CD28 reagents (Transact, 130-111-160 Miltenyi) and 40 IU/ml 112 (PCH0021, Gibco) and between 0 and 200 μg/ml CAR-NLP for one (1) day, followed by washing three (3) times with PBS+1% BSA, staining with PI and FACS to determine viable cell fraction. Viability changes <20% across 0-200 μg/ml doses.

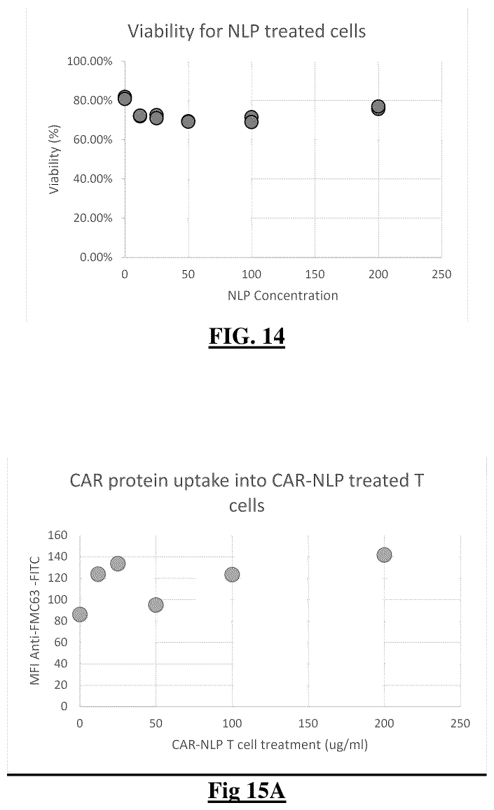

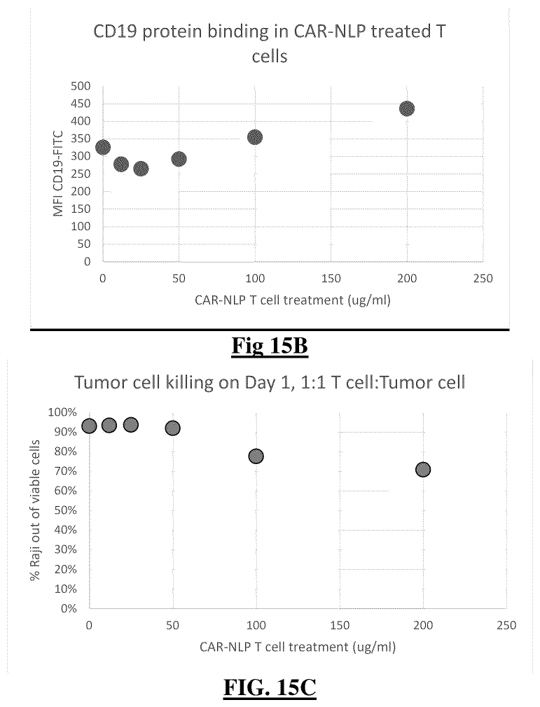

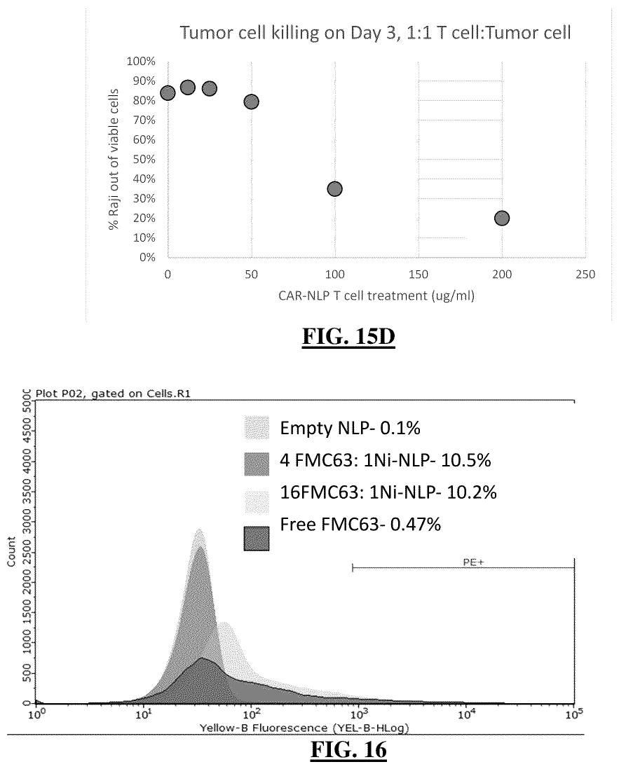

Results of additional experiments are reported in FIGS. 14, 15A 15β, 15C and 15D.

FIG. 14 shows that altering formulations can improve viability issues for human peripheral blood T-cells. By careful removal of imidazole and insoluble protein (through cell-free expression at low temperature), minimal (<10%) changes in cell viability can be observed. Importantly, these cells retain high levels of viability across a broad dose range of nanoparticle concentrations, with minimal cytotoxicity observed up to 250 μg/mL, indicating the CAR-NLP formulations are well tolerated by primary human T cells under the tested conditions.

FIG. 15 presents a comprehensive evaluation of CAR-NLP functional performance across several key cellular metrics, including viability, CAR surface expression, antigen binding, and tumor cell cytotoxicity.

FIG. 15A presents a dose-response curve showing the expression of chimeric antigen receptor (CAR) protein on the surface of T cells following incubation with CAR-NLPs. CAR expression is quantified via mean fluorescence intensity (MFI) using an anti-FMC63-FITC antibody specific for the scFv domain of the CAR. The results confirm that CAR-NLPs mediate dose-dependent cell surface expression of CAR protein, with saturation occurring at higher nanoparticle concentrations.

FIG. 15B presents a dose-response curve showing CD19 binding, i.e. function, of chimeric antigen receptor (CAR) protein on the surface of T cells following incubation with CAR-NLPs. CAR function is quantified via mean fluorescence intensity (MFI) using CD19 protein-FITC chimeric protein that represents the ligand of the CAR. The results confirm that CAR-NLP transferred CAR protein on the surface of T cells can mediate CD19 protein binding indicating functionality in the cellular environment.

FIG. 15 Cand FIG. 15D show that T-cells treated with CAR-NLP formulated to remove imidazole and insoluble protein can induce T-cells to recognize and kill tumor cells. T-cells were treated with CAR-NLP at doses between 0 and 200 μg/ml overnight in the presence of anti-CD3/CD28 activation reagents, then washed and mixed with Raji tumor cells at 1:1 ratio.

In particular, a fraction of Raji from total cells was analyzed by flow cytometry after one (1)(FIGS. 15C) and three (3) days (FIG. 15D). Decreasing fraction of Raji in the total viable cell fraction indicates antitumor cytotoxicity.

The results shown in FIGS. 15 Cand 15D demonstrate tumor cell killing which represents the most important data point and typically indicates that T cells take on cytokine expression. As we have shown that we can transfer CAR to multiple immune cell populations, it is reasonable to expect transfer of tumor cell recognition and immune cell activation in other immune cell populations

Despite the relatively modest CAR expression and CD19 binding observed in FIGS. 15B and 15C, the CAR-NLP-treated T cells display a marked increase in tumor killing activity by Day 3, indicating that even sub-saturating levels of functional CAR protein can mediate significant cytolytic responses over time.

These findings collectively demonstrate that CAR-NLPs, while exhibiting relatively poor uptake as measured by CAR surface staining and antigen binding, are nevertheless capable of reprogramming T cells to exert potent and time-dependent tumoricidal activity. This underscores the functional relevance of even modest levels of NLP-mediated CAR delivery and supports the utility of the platform for applications where transient, non-genomic CAR expression is desired.

These effects of insoluble protein and imidazole purification buffers on T cell viability have not previously been identified as result affective variables for NLP mediated protein delivery.

### Example 7. NLP Mediated CAR T-Cell Engineering Generates Functional T-Cells

Immune cells such as T-cells play a crucial role in pathogen clearance as they kill infected cells and coordinate inflammation [60]. A crucial, non-obvious demonstration of CAR-NLP protein engineering of immune cells is in vitro cytotoxicity.

ApoA1 and CAR were co-expressed at a 20:1 plasmid ratio in the presence of 2 μg/ml DMPC in 5×Biotech rabbit E. Coli cell-free lysate 1 ml reactions in a reaction chamber, at 20° C. for 18 hours. Reaction products were purified using nickel resins, dialyzed into PBS, centrifuged at 14 kg to remove insoluble material, and then filter sterilized through a 0.2 μm PES syringe filter.

Total protein concentration (CAR+Apo) was 223 μg/ml and the CAR protein alone was 8.6 μg/ml. NLPs were then concentrated 2f-old in a centrifugal concentrator.

Human peripheral blood T-cells sorted by negative selection were thawed to resting culture in T-cell medium (Tecsmax, Miltenyi) at 1million cells/ml and rested overnight in a humidified incubator at 37° C. and 5% CO2. Cells were stimulated with 1:50 anti-cd3/cd28 reagent (Transact, Miltenyi), and 40 U/ml recombinant human interleukin-2 plus between 0 and 200 μg/ml CAR-NLP suspension (200 μg/ml corresponds to 4.5 micromolar nanodisc and 7.6 μg/ml CAR or 95nanomolar CAR).

Nanodisc mediated CAR delivery was assessed by mixing cells with FITC tagged CD19 protein for 30 minutes on ice, then counting the FITC mean free intensity for viable cells

For demonstrating full nanodisc mediated CAR T engineering functionality, we collected cells treated with CAR nanodiscs for 24 hours and mixed at a 1:1 ratio with CD19 expressing Raji tumor cells. At one (1) and three (3) days post co-culture, the number of viable Raji cells were counted.

The results reported in FIGS. 13A and 13B show the delivery result in a T-cell population with at least 2% of cells with detectable receptor on the cell surface.

### Example 8. CAR-NLP Engineering of Immune Cells

Experiments were performed to test NLP protein transfer on the membrane of immune cells.

A first set of results reported in FIG. 16 shows that conjugating an scFv to an NLP permits efficient delivery to T cells, whereas free scFv in the absence of NLP components is not taken up into T cells.

A second set of results reported in FIG. 17A to 17F show that CAR-NLP engineering can be accomplished using the methods of the present disclosure in both CD4+ and CD8+ T cells, B cells, and NK cells.

### Example 9. Low Temperature Synthesis Improves Soluble Yield

Beta2 adrenergic receptor delta70 and ApoA1 were co-expressed at 15:1 ratio in 1 ml of Biotech rabbit E. Coli cell free lysate in a reaction chamber with the addition of 2 mg/ml DMPC at either 20° C. of 30° C. for either six (6), 18, 24, or 48 hours.

NLPs were purified from the reaction mix using nickel affinity resins and eluted with imidazole buffer, then fractions with high concentrations were pooled.

The resulting NLPs suspension was then dialyzed against PBS. Insoluble material was removed by centrifugation at 14,000g, then the total and soluble fractions were run on PAGE to analyze protein density.

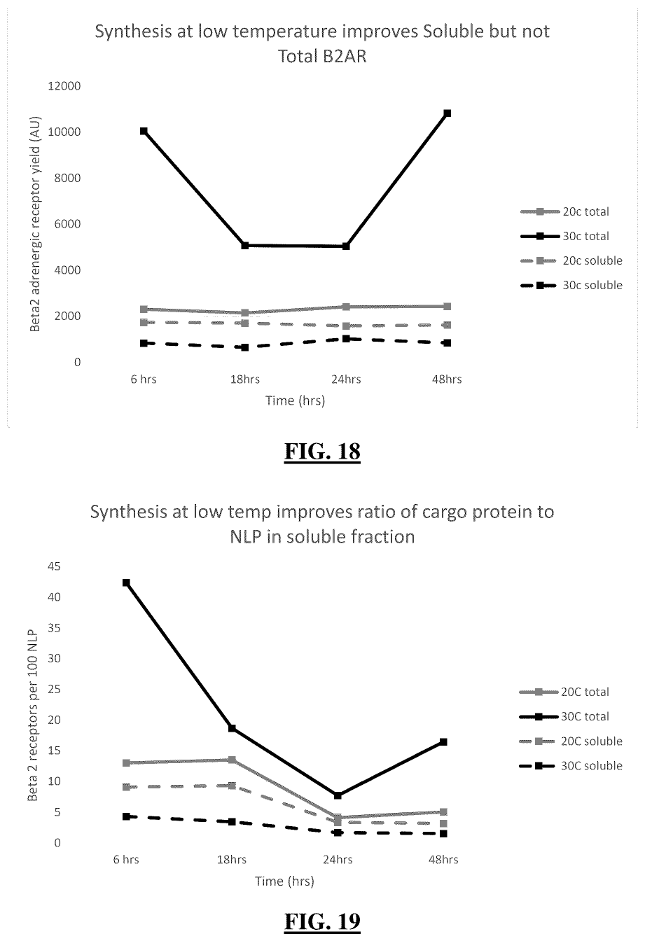

The results are shown in FIG. 18, which shows that the total protein at all studied timepoints was significantly higher for B2AR-NLP synthesized at 30° C. compared to 20° C. However, comparing soluble protein concentration showed the exact opposite effect, i.e., the soluble protein concentration was higher in the 20° C. synthesized mix as compared to 30° C. Therefore, at all timepoints, synthesis at 20° C. resulted in significantly higher solubility (60-80%) compared to synthesis at 30° C. (7-20%).

As shown by the exemplary data of FIG. 18, synthesis at 30° C. resulted in significantly higher total protein concentration, however the solubility was poor (8-20%). While synthesis at 20° C. resulted in much less total protein, the increased solubility (70-80%) resulted in a higher soluble protein concentration than at higher temperature.

The relationship between synthesis temperature and cargo protein loading in the total and soluble protein fractions shown by the data is reported in FIG. 19.

FIG. 19 shows a chart illustrating that the ratio of beta-2 adrenergic receptors per NLP is higher in the NLPs synthesized at 30° C. as compared to at 20° C., however this material is poorly soluble. Removing the insoluble fraction reveals that the cargo loading in the 20° C. synthesis is >2-fold higher than in the 30° C. synthesis at all timepoints investigated.

### Example 10 Design of Chimeric Antigen Receptor (CAR) and Apolipoprotein Plasmids

The pSLCAR-CD19-282-pIVEX2.3d plasmid was generated by codon optimization (GenSmart Codon Optimization Tool) of a previously published CAR construct (pSLCAR-CD19-28° C.) that targets CD19 (kind gift of Scott McComb, Addgene plasmid #135991) for bacterial expression. This gene was then synthesized in a pIVEX2.3 backbone (Rabbit biotech, BR1400701) that includes the T7 promoter sequence necessary for expression in cell-free E. coli lysates.

The apolipoprotein pIVEX2.4d plasmid encoding a 5×histidine tagged truncated apolipoprotein Al (A1-49) was generated in previous work (Cappuccio et al Molecular and cellular proteomics Volume 7, Issue 11p2246-2253November 2008) All plasmids were verified by sequencing, transformed into E. coli (ThermoFisher, C₄₀₄₀₁₀), and purified by maxiprep (Qiagen, Cat #12165). Purified plasmids were stored at-200C until further use.

### Example 11 Lipid Preparation

Lipids (DPMC, DMPG, POPC, EggPC in Table 1) were reconstituted from powder with UltraPure DI water (Invitrogen, 10977015) at a concentration of 20 mg/mL. Lipids were then prepared into small unilamellar vesicles (SUVs) by probe sonication (qSonica, Q500-110) and a ⅛th inch probe tip (qSonica, #4418), using a pulse amplitude of 22% and 15 second on-off cycles for a total of 2 minutes, representing a total energy input of 600-700 joules.

TABLE 1
Lipids Used in CAR-NLP Production

| | | Catalog | |
|---|---|---|---|
| Lipid Name | Abbreviation | number | Manufacturer |
| 1,2-dimyristoyl-sn-glycero-3-phosphocholine | DMPC | 860345P | Avanti Polar |
| | | | Lipids |
| 1,2-dimyristoyl-sn-glycero-3-phospho-(1′- | DMPG | 840445P | Avanti Polar |
| rac-glycerol) | | | Lipids |
| 1-palmitoyl-2-oleoyl-glycero-3- | POPC | 850457 | Avanti Polar |
| phosphocholine | | | Lipids |
| L-α-phosphatidylcholine (Egg, Chicken) | EggPC | 840051 | Avanti Polar |
| | | | Lipids |

The sonicated lipid solution was then centrifuged at 10,000 rcf for 1 minute to remove insoluble materials and probe tip debris. SUVs were collected by transferring the supernatant to a clean 1.5 mL microcentrifuge tube (Eppendorf, 022364111) and stored at room temperature for future use in cell free reactions.

### Example 12 Synthesis of CAR-NLP with a Kit Based Cell Free lysate

CAR protein loaded Nanolipoprotein particles were co-expressed through cell-free methods as previously described [61] using commercial reaction mixes (RTS 500 Proteomaster E. Coli HY kit, Biotechrabbit GMbH, BR1400201). Reaction and feeding mix components were thawed and reconstituted according to manufacturer's instructions.

For CAR loaded NLPs, 20 μg CAR-CD19-28z plasmid, 1 μg Δ49ApoA1 plasmid, and 2 mg total lipid were added to the reaction mixture then the mix was incubated while shaking. For empty NLPs, 1 μg Δ49ApoA1 plasmid and 2 mg total lipid were added to the reaction.

For nascent protein tracking, Bodipy dye (Fluoro Tect GreenLys in vitro Translation Labeling System, Promega, L5001) was added to each reaction mixture at 1:200 (v/v).

### Example 13: CAR-NLPs Purification

CAR-NLPs or empty NLPs were purified using affinity chromatography in gravity flow columns according to manufacturer's specifications. Briefly, columns (BioRad, 7311550) were prepared by loading 1 mL nickel agarose (complete His-Tag Purification Resin Roche, 08778850001) and washing the resin with 3 mL of wash buffer.

Unpurified cell free reaction products in 250 μL of 10 mM lysis buffer were loaded on the column for 1 hour at 40C with shaking on nutator. Following incubation, columns were washed six times with 1 mL of wash buffer (Table 2), then eluted using six 300 μL washes of elution buffer (Table 3) and one final 300 μL wash with final elution buffer (Table 2).

TABLE 2
NLP Purification Buffer Compositions

Stock Information

| | Sigma | Honeywell | Sigma | |
|---|---|---|---|---|
| | S9638- | BJS9888- | I2399- | |
| | 1 KG | 10 KG | 500 G | |
| Buffer Name | NaPO4 | NaCl | Imidazole | pH |
| Lysis Buffer | 50 Mm | 300 mM | 10 mM | 8.0 |
| Wash Buffer | 50 mM | 300 mM | 20 mM | 8.0 |
| Elution Buffer | 50 mM | 300 mM | 250 mM | 8.0 |
| Final Elution | 50 mM | 300 mM | 500 mM | 8.0 |
| Buffer | | | | |

Eluted fractions were analyzed by SDS-PAGE as Fractions containing desired NLP products were pooled and analyzed again using quantitative PAGE. Purified NLPs were dialyzed into 4L PBS with 2 buffer exchanges for 2-16 hr at 40C using dialysis chambers with a 3.5 kDa molecular weight cutoff (D-Tube Dialyzer Maxi, Millipore-Sigma,71508-3). Following dialysis, samples were sterile filtered through 0.2 μm PES filters (Millipore-Sigma, SLGPR33RS).

### Example 14. Lyophilization and Storage of CAR NLP

NLP aliquots were pipetted into sterile 15 mL tubes or 1.5 mL Microcentrifuge tubes at a volume as to not exceed 1/3 of the container. Sterile, 1M Trehalose was added to the samples at a 1:10 ratio.

Samples were frozen on dry ice for a minimum of 30 minutes, then lyophilized for a minimum of 12 hours (FreeZone 2.5 L−84° C. Benchtop Frecze dryer, Labconco 710201015)

### Example 15 SDS-PAGE Analysis of CAR-NLPs

For SDS-PAGE analysis, 5 μL of CAR-NLP or Empty NLP samples were mixed with 10 μL sample buffer containing DTT (ThermoFisher NP0008, ThermoFisher, NP0009 and DI water) and denatured at 95° C. for 5 minutes. 15 ul of prepared samples were loaded into 4-12% gradient Bis-Tris Gels (ThermoFisher, NP0322/NP0323/NP0329) with a lane reserved for ladder (Novex Sharp Pre-stained Protein Standard). Samples were electrophoresed at 200V for roughly 35 minutes (Invitrogen™ XCell SureLock Mini-Cell, EI0001)

Following electrophoresis, gels were imaged at 488 nm for Bodipy stained nascent protein (LI-COR Odyssey M Imager LICORbio, 3340). Total protein was analyzed by staining with either SYPRO Ruby (SYPRO Ruby Protein Blot Stain, ThermoFisher, S11791) or the Coomassie blue (eStain L1 Protein Staining System, Genscript, L00657/L00753) according to manufacturer's instructions, then imaged at 488 nm.

For analysis of soluble protein, purified solutions were centrifuged for 10 minutes at 25,000g and supernatant was collected for analysis of soluble protein. Total protein was analyzed without centrifugation. For quantitative PAGE, samples were run in parallel with the bovine serum albumin standards: 1 μg, 0.5 μg, 0.25 μg, 0.125 μg, and 0.0625 μg diluted in 1×PBS (ThermoFisher, 23209).

Following imaging, protein band fluorescent intensity was measured using Fiji software as previously described [. Band intensities were compared by one-way ANOVA, then plotted using GraphPad Prism.

### Example 16: Cell Free Expression of CAR-NLP with Known Methods Results in High Insoluble Fraction

Cell free methods for formation of nanolipoprotein particles have been shown to improve both solubility and yield of large transmembrane proteins [61, 62])

Experiments were performed to evaluate the yield and solubility of CAR-NLPs produced with commercial kits and known methods without the modifications and optimization of the methods and systems to produce immunonanolipoprotein particles herein described.

The preparation of plasmids for expression of chimeric antigen receptor (CAR) and apolipoprotein proteins involved cloning into a pIVEX2.3d backbone suitable for E. coli cell-free expression as described in Example 10. The apolipoprotein plasmid encoding a His-tagged, truncated form of ApoA1 (41-49) was previously prepared in a pIVEX2.4d backbone. Both plasmids were sequence-verified, transformed into E. coli, and purified using commercial maxiprep kits. Resulting plasmid DNAs were stored at −20° C. for future use in in vitro transcription/translation systems. Small unilamellar vesicles (SUVs) of DMPC were also prepared in parallel by probe sonication of reconstituted lipid powders, followed by clarification by centrifugation as described in Example 11

Nanolipoprotein particles (NLPs) co-loaded with CAR and ApoA1 were synthesized using a commercial cell-free protein synthesis kit, where 20 μg of CAR plasmid, 1 μg of apolipoprotein plasmid, and 2 mg of lipid were incubated in reaction mix under screening conditions as described in Example 12 Empty NLP controls omitted the CAR plasmid.

After cell-free expression, NLPs were purified via Ni-NTA affinity chromatography under cold conditions, followed by dialysis into PBS, sterile filtration as described in Example 13, and optional lyophilization with trehalose as a cryoprotectant as described in Example 14.

To assess NLP composition and expression efficiency, SDS-PAGE was performed on purified products as described in Example 15. Samples were denatured with DTT-containing buffer, resolved on 4-12% gradient Bis-Tris gels, and imaged at 488 nm to detect fluorescently labeled proteins. Total protein content was further visualized with SYPRO Ruby or Coomassic staining. Solubility was assessed by centrifugation prior to gel analysis. Quantitative PAGE using BSA standards enabled estimation of protein yield, and band intensities were quantified using Fiji, followed by statistical comparison using ANOVA and GraphPad Prism plotting.

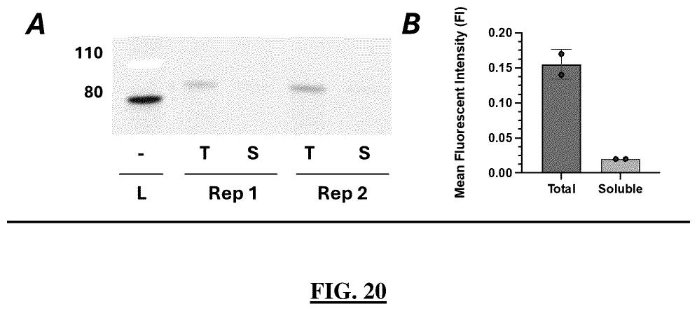

The results illustrated in FIG. 20 show that the production of CAR NLPs following these methods resulted in fraction of highly insoluble NLPs.

In particular, FIG. 20 Panel A: shows the SDS PAGE of yield from cell free synthesis of CAR protein which shows that in both replicate experiments the soluble fraction was very low (see FIG. 20 Panel A S band) compared with insoluble fraction (FIG. 20 Panel A T Band).

The difference between soluble and insoluble fraction obtained was quantitively detected by band densitometry and the results reportd in FIG. 20 Panel B confirming that insoluble fraction of synthesized CAR protein is significantly higher than the soluble fraction. The results illustrated in FIG. 20 Panel B thus confirm the indication of the SDS PAGE of FIG. 20 Panel A showing that while protein synthesis could be detected, soluble yield was extremely poor, and almost indistinguishable from background.

### Example 17. Production and Optimization of Solubilized CAR Protein Expression Lipid Composition Cell-Free NLP Assembly

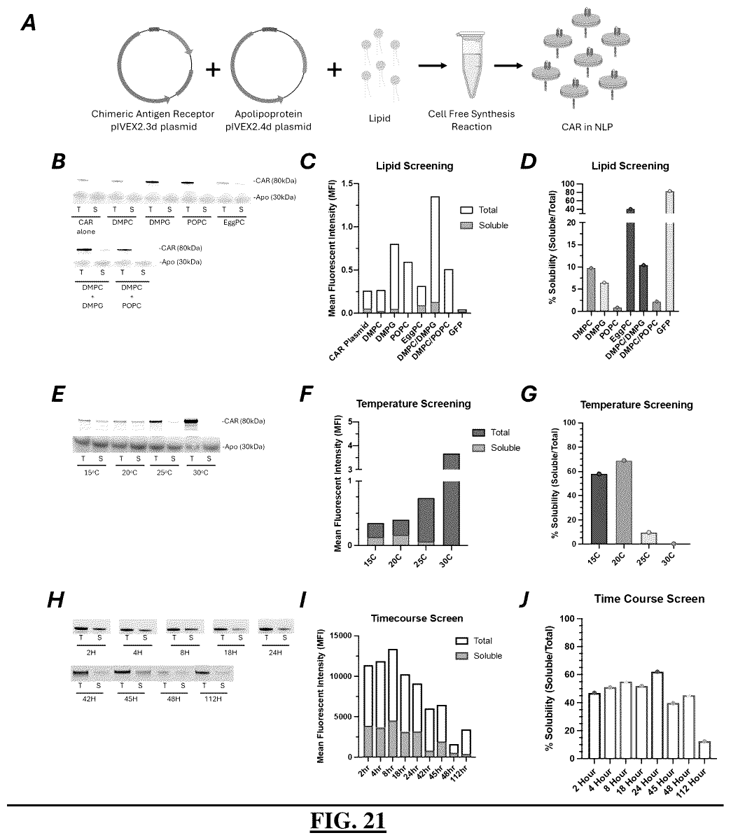

To address the low yield and solubility of CAR proteins, we co-expressed CAR and Apolipoprotein-Al (Apo) using three methods to produce immunonanolipoprotein particles of the present disclosure according to the step schematically shown in FIG. 21 panel A and the procedure described in Example 5.

A series of optimization screens was then performed to verify optimization of lipid composition used during the immunoNLP assembly

Experiments were performed by first comprising CAR and Apo yield with lipid choice as described Table 1 and Example 11 Lipid compositions tested included DMPC, DMPG, POPC, EggPC, and a DMPC/DMPG blend. Resulting protein products were evaluated by SDS-PAGE followed by fluorescent densitometry quantification of both total and soluble fractions.

To this purpose, CAR-NLPs were assembled in the presence of 2 mg/ml reaction of different lipids compositions with the materials and methods described in Examples 10 to 14 and the result evaluated by SDS-PAGE performed according to the procedures described in Example 15.

The results illustrated in FIG. 21, Panels B-D illustrate the effect of lipid composition on the solubility of a chimeric antigen receptor (CAR) protein co-expressed with a truncated apolipoprotein in a cell-free system. Panels include representative SDS-PAGE gels and densitometric quantification of total and soluble protein yields under different lipid and temperature conditions.

As shown in in FIG. 21, Panels B-D inclusion of lipids generally improved both total and soluble CAR yield compared to CAR and Apo alone, and that the specific lipid composition affected the total CAR yield and soluble CAR yield. Apo expression was insensitive to lipid.

The results reported in FIG. 21, Panels B-D a subset of lipid conditions supported appreciable solubility of the expressed CAR protein. DMPC, EggPC, and DMPC/DMPG conditions yielded >10% soluble CAR protein, with DMPC giving the highest overall soluble yield. Apo expression was comparatively unaffected by lipid composition. These results indicate that CAR synthesis and solubility is sensitive to lipid mix, and optimizing CAR yield requires considering lipid composition

These findings support the conclusion that lipid content is a result effective variable of the production of immune-NLPs and that rational lipid selection can be used to solubilized NLP integration of membrane proteins under defined, cell-free reaction conditions.

These findings demonstrate that both purified lipids and natural lipid mixes can be used to prepare immune NLPs, and that saturated zwitterionic lipids outperform negatively charged lipids for solubility. Mixtures appear to perform less well than pure species . . .

### Example 18. Production and Optimization of Solubilized CAR Protein Expression of Reaction Temperature in Cell-Free NLP Assembly

A series of optimization screens of the exemplary method to manufacture immune-NLPs herein described in Example 5, was then performed to verify optimization of reaction d temperature of the immunoNLP assembly

Experiments simultaneous run according to the materials and methods reported in Examples 10 to 15, in which different reaction temperatures were also screened for effects on solubility of the CAR-NLPs using DMPC lipids.

The results illustrated in FIG. 21, Panels E-G illustrate the effect of lipid composition and incubation temperature on the solubility of a chimeric antigen receptor (CAR) protein co-expressed with a truncated apolipoprotein in a cell-free system. Panels include representative SDS-PAGE gels and densitometric quantification of total and soluble protein yields under different lipid and temperature conditions.

In particular, increasing reaction temperature showed opposite effects on total protein produced and soluble protein produced (FIG. 21, Panel F). Reactions run at 15 C and 20° C. resulted in less total protein produced compared to reactions run at 25° C. and 30° C. (FIG. 21, Panels F and G). However, lower temperature reactions resulted in nearly 5×and 7×more soluble protein respectively, compared to reactions ran at 25° C. Furthermore, while reactions at 30° C. produced the highest total protein of the four conditions, this reaction resulted in less than 1% of the protein being soluble and suggesting protein was degraded.

According the results reported in FIG. 21, Panel s E-G show that temperature inversely affected total versus soluble protein yield. While 30° C. yielded the highest total CAR protein, the soluble fraction was negligible (<1%). In contrast, 20° C. supported an optimal combination of yield and solubility, with 5-7-fold higher soluble CAR protein than higher temperatures.

These findings support the conclusion that rational lipid selection and low-temperature synthesis enable solubilized NLP integration of membrane proteins under defined, cell-free reaction conditions.

Thus synthesis at temperatures under the transition temperature of the lipid included in the reaction mixture may improve soluble yield by more closely or more rigidly interacting with newly synthesized insoluble transmembrane domains. Notably, admixtures of lipids tend to have a higher transition temperature than either pure species [63], resulting in poorer solubility at the same temperature. Soluble yield may be improved by reducing further the synthesis temperature when lipid mixtures are desired.

### Example 19. Optimization of CAR-NLP Expression reaction times

A series of optimization screens of the exemplary method to manufacture immune-NLPs herein described in Example 5, was then performed to verify optimization of reaction time of the immunoNLP assembly

Building on the solubility-optimized conditions described in Examples 17 and 18, we conducted a time-course experiment to identify the temporal window supporting maximal soluble protein incorporation into NLPs.

Using DMPC as the lipid phase and maintaining reaction temperature at 20° C., reactions were sampled across a range of timepoints (2-112 hours).

In particular, reaction time was screened, using 20° C. reaction temperature and DMPC lipids for 2-, 4-, 8-, 18-, 24-, 42-, 45-, 48-, and 112-hours.

The time-course experiment was performed evaluating total and soluble protein expression over a range of incubation durations for reactions conducted at 20° C. in the presence of DMPC lipid vesicles. Representative SDS-PAGE gels and quantitative plots are included to identify an optimal expression time for solubilized CAR-NLP production.

The results reported in FIG. 21 Panels H-J show total protein yield was highest at 8-hours, followed closely by the 4-hour reaction timepoint, whereas 24-hour reactions showed less total protein produced, but an increase in soluble protein compared to 4- and 8-hour (FIG. 21 panel J).

Accordingly, the results of FIG. 21 Panels H-J indicate total protein peaked at approximately 8 hours, while the proportion of soluble protein increased steadily to a maximum at 24 hours. Thereafter, both total and soluble yield declined, indicating potential degradation or aggregation of the CAR protein product . . .

This analysis informed the selection of a 24-hour reaction window as a preferred duration for production of NLP-integrated membrane proteins in a soluble, functional state. These results further support the use of defined lipid and temporal conditions to promote assembly of supramolecular NLP complexes with embedded target proteins.

Following completion of the reaction conditions screen, DMPC, 20° C. and 24-hour reaction times was selected for all subsequent experiments reported in the Examples 26

### Example 20 Synthesis of CAR-NLP with Custom-made Cell-Free (H-CF) Lysate

Custom-made Cell-free protein synthesis lysates were prepared according to methods adapted from previous work

Briefly, E. coli (ClearColi, Bio Search Technologies) were grown in 2xYT media supplemented with 0.5% NaCl. T7 polymerase expression was induced with 1 mM IPTG before harvesting. Cells were collected, washed with Buffer A (10 mM Tris base, 14 mM Magnesium Glutamate, 60 mM Potassium Glutamate, 1 mM DTT), and then resuspended at 1 ml/gram wet mass before freezing.

Thawed cells were sonicated (qSonica Q500 with probe tip sonicator), and centrifuged for 10 min at 18,000rcf and 4° C. Supernatants were incubated at 37° C. for 30 min for the runoff reaction before centrifuging for 10 min at 10,000rcf and 4° C. Supernatants then were collected, frozen, and stored at −80° C. for future use in cell free reactions.

H-CFPS reactions were performed using the PanOx-SP reaction buffer system [15007837] with the following added components: 1.2 mM ATP, 0.86 mM GTP, 0.86 mM UTP, 0.86 mM CTP, 170 μg/mL E. coli tRNA, 2 mM of each amino acid (except glutamate), NAD 0.33 mM, 0.27 mM Acetyl-CoA, 1.5 mM spermidine, 1 mM putrescine, 175 mM potassium glutamate, 10 mM ammonium glutamate, 10 mM magnesium glutamate, 33 mM PEP, 0.5 mM DTT, and 25% v/v lysate.

Plasmid DNA was added at a concentration of 20 μg/mL reaction and 2 mg/ml total lipid were added to the reaction. H-CFPS reactions times and temperatures were: 18 hours at 15, 20, 25 or 30° C. or 2, 4, 8, 18, 24, 42, 45, 48, or 112 hours at 25° C. For nascent protein tracking, Bodipy dye (Fluoro Tect GreenLys in vitro Translation Labeling System, Promega, L5001) was added to each reaction mixture at 1:200 (v/v).

### Example 21. Western Blots of CAR NLPs

Following SDS-PAGE as above, western blot transfers were performed using a dry transfer system (iBlot 2, Invitrogen, IB21001) following the manufacturer's protocol. Briefly, gel and a nitrocellulose membrane were loaded into the apparatus and transferred for roughly 7 minutes. The nitrocellulose membrane was then blocked at 4C for Ihr in 10 mL blocking buffer (Blocking Buffer, LICORbio, 927-70001).

After blocking, membranes were then washed 4 times in 5 mL 1×PBS+0.5% Tween (PBST) for 1 minute, while rocking at room temperature. The membranes were then stained with primary antibody solution (Table 3) for 1 hour while rocking at room temperature followed by 4 PBST washes, then incubated in secondary antibody solution (Table 3) for 1 hour at RT, while protected from light, followed by 4 PBST washes, and one wash with 5 mL of 1×PBS.

TABLE 3
Membrane Staining

| Antibody | Antibody | | MFG, | | | | Incubation | Incubation |
|---|---|---|---|---|---|---|---|---|
| Type | Target | Clone | Stock # | Dilution | Volume | Buffer | Temperature | Time |
| Primary | mouse α FLAG | Monoclonal | Millipore | 1:1000 | 8 | LICOR Blocking | RT | 1 H |
| | M2 | | Sigma, | | mL | Buffer + 0.2% | | |
| | | | F1804 | | | Tween | | |
| Primary | aHIS | | Qiagen, | 1:700 | 8 | LICOR Blocking | RT | 1 H |
| | | | 34660 | | mL | Buffer + 0.2% | | |
| | | | | | | Tween | | |
| Secondary | IRDye 800CW | Polyclonal | LICOR, | 1:10,000 | 7.5 | LICOR Blocking | RT | 1 H |
| | Goat Anti-Mouse | | 926-32210 | | mL | Buffer + 0.2% | | |
| | IgG | | | | | Tween | | |

Membranes were imaged at 800 nm (Odyssey M Imager, LICOR, 3340). Gels were analyzed using Fiji software and band intensities were compared using one-way ANOVA, then plotted using GraphPad Prism.

### Example 22. Cell Culture and Cell Lysate Production

Raji (ATCC, CCL-86) and K562 (ATCC, CCL-243) were cultured according to manufacturer's specifications. Briefly, Raji were seeded into flasks at 4x10⁵ cells/ml and subcultured at 3x10⁶ cells/ml in RPMI-1640 (ATCC, 30-2001) with 10% fetal bovine serum (ATCC, 30-2020). K562 were seeded at 1x10⁵ cells/ml and subcultured at 1 ×10⁶ cells/ml in IMDM (ATCC, 30-2005) with 10% fetal bovine serum (ATCC, 30-2020).

Cells were maintained at 370C in humidified incubators with 5% CO2. K562 were transfected with a CD19 overexpression plasmid, pCDH-CMV-CD19 puro (a gift from Nicolas Manel, Addgene plasmid #196634) then selected using 5 μg/ml puromycin (Gibco, A1113802). CD19 expression was confirmed by flow cytometry (BDFACS Celesta) using an anti-human CD19-AF647 antibody (Biolegend, 302220, clone HIB19).

For cell lysates, 1 million cells were collected, pelleted by centrifugation, washed with sterile 1×PBS (Thermofisher 70011069) then lysed using cold RIPA buffer (ThermoFisher, 89900) with added protease inhibitors (Roche complete EDTA free protease inhibitor cocktail, 4693132001) for 30 minutes on ice. Lysates were clarified by centrifugation at 16,000g and supernatants were then frozen at-800C. Flow cytometry results were analyzed using FlowJo software and plotted using GraphPad Prism.

### Example 23: Dot Blot Analysis of CAR-NLPs

A nitrocellulose membrane was pre-wet for 5 minutes with 1×PBS buffer, then placed into a Bio-Dot Apparatus (Bio-Rad, 1706545) and wells were washed with 20 uL 1×PBS buffer. The membrane was then loaded in duplicate with either 10 ug Empty NLP from the R-CF reaction, CAR-NLP from the R-CF reaction, or CAR-NLP from H-CF reaction lysates, for 20 minutes at room temperature. The membrane was removed from the apparatus and washed with 5 ml PBST for 1 minute, while rocking at room temperature.

The membrane was blocked in a 5% non-fat dry milk (BIO-RAD, 1706404XTU) in PBST for 1 hour while rocking at room temperature. Following incubation, the membrane was washed 4 times with 5 mL PBS. Following washes, the membrane was incubated overnight at 4° C. while shaking with the 1°

Antibody binding solution (Table 4)], followed by 4 PBST washes and incubation into 2° Antibody binding solution (Table 4), for 1 hour at RT, followed by 4PBST washes and incubation with the 3° Antibody binding solution (Table 4) for 1 hour at RT. Membranes were washed 4 times for 5 minutes and then one time briefly with 1×PBS to remove detergent. Blots were then imaged at 800 nm (LI-COR Odyssey M Imager

TABLE 4
Primary, Secondary, and Tertiary Dot Blot Staining Solutions

| | | Antibody | | MFG, Stock # | | Incubation (at | |
|---|---|---|---|---|---|---|---|
| Corresponding | | binding | Solution | (if | Dilution/ | room temp, in | |
| Sample | Condition | step | Composition | Applicable) | Concentration | 5% PBST | |
| All Samples | Experimental | Probe | CAR-NLP | N/A | 10 ug/ | 10 | min |
| | | | | | spot | | |
| All Samples | Experimental | Probe | Empty-NLP | N/A | 10 ug/ | 10 | min |
| | | | | | spot | | |
| All Samples | Control | Probe | His-Tagged | Acrobio, CD9- | 1:500 | 10 | min |
| | | | FMC63 scFv | M52Hb | | | |
| | | | Antibody | | | | |
| CAR-NLP, | Experimental | Primary | Anti-FLAG | ID: F1804- | 1; 1000 | 1 | HR |
| Empty-NLP | | | | 200UG Lot# | | | |
| | | | | 00000308215 | | | |
| His-Tagged | Control | Primary | Anti-His | ID: 346690 | 1:700 | 1 | HR |
| FMC63 scFv | | | | | | | |
| Antibody | | | | | | | |
| All Lysates | Experimental, | Secondary | Goat-Anti | ID: 926-32210 | 1:10,000 | 1 | HR |

| | | |
|---|---|---|
| Control | Mouse 800 | Lot#D30725- |
| | | 05 |

LICORbio, 3340). Gels were analyzed using Fiji software and differences in band intensities were determined by one-way ANOVA, then plotted using GraphPad Prism.

### Example 24. CAR-NLP and FMC63 Bead Binding Assay

Dynabeads His-tag beads (ThermoFisher, 10103D) vortexed prior to use and 10 ul was transferred into a

1.5 mL microcentrifuge tube. Beads were washed 3× with 500 μl of PBS on a magnetic rack according to manufacturer's specifications. Following washing, beads were loaded with either 200 ug CAR-NLP, 200 ug Empty-NLP or 20 ug FMC63 His-Tagged Protein (Acro Biosciences, CD9-M52Hb) by mixing for 30 minutes at 40C with a nutating mixer.

Following incubation, tubes were placed on a magnetic rack, supernatant was removed, and beads were washed 3 times with 1 mL of RPMI-1640 supplemented with 10% FBS (ATCC 30-2001). Beads were then mixed with 100,000 Raji cells and incubated for 30 minutes at 40C with rocking. Beads were then washed 3 times as before. Cells were imaged and beads attached to each cell was counted using a microscope. Raw data was analyzed and then plotted using GraphPad Prism.

### Example 25: Codon optimization of CAR construct

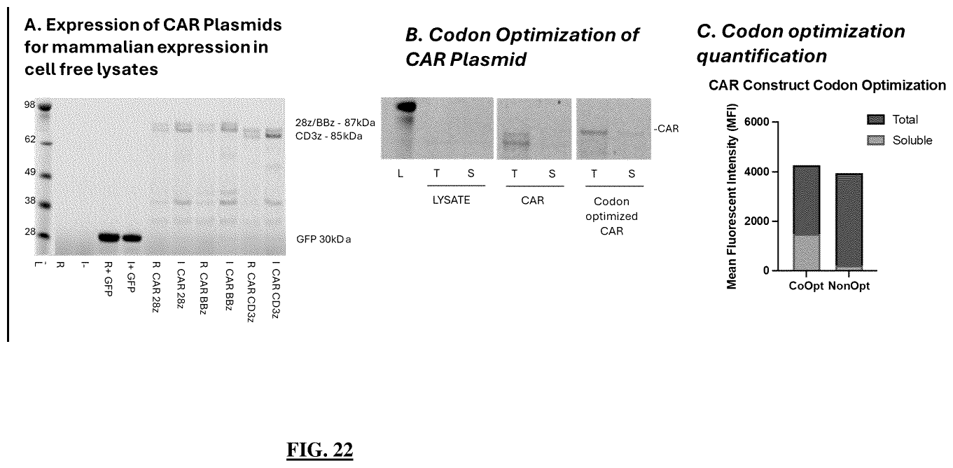

A set of experiments were performed to assess whether using Cell Free (CF) protein production methods could generate CAR protein using a CAR designed for mammalian expression) in a plasmid for CF expression and found that trace protein could be expressed (FIG. 22 Panel A).

Codon optimization of this plasmid for E. coli expression resulted in higher total protein expression compared to the unoptimized mammalian expression plasmid (FIG. 22 Panels B and C). Additionally, codon optimization resulted in a single band unlike the mammalian expression construct which appeared to form multiple bands, potentially indicating improved quality.

Repeated runs using the codon optimized vector resulted in similar yields, but the resulting material is highly insoluble across all runs (FIG. 22 Panel C).

### Example 26: CAR-NLP Production and Purification-Lysate Optimization

CAR-NLPs were produced in milliliter scale production reactions comparing commercially available lysates (R-CF) to lysates produced in house (H-CF).

Under the optimized expression conditions described in Examples 17-19 CAR-NLPs were synthesized and purified at preparative scale using either a commercial cell-free lysate system (R-CF) or a homemade lysate (H-CF). Purified products were analyzed for incorporation of both CAR and Apo proteins. To this end, following production using optimized conditions DMPC lipid, 20° C. and 24-hour reaction, purification of the CAR-NLPs was completed using affinity resins targeting Apo. Visualization of the purified products indicated that CAR protein was successfully embedded within the NLP in the final preparations.

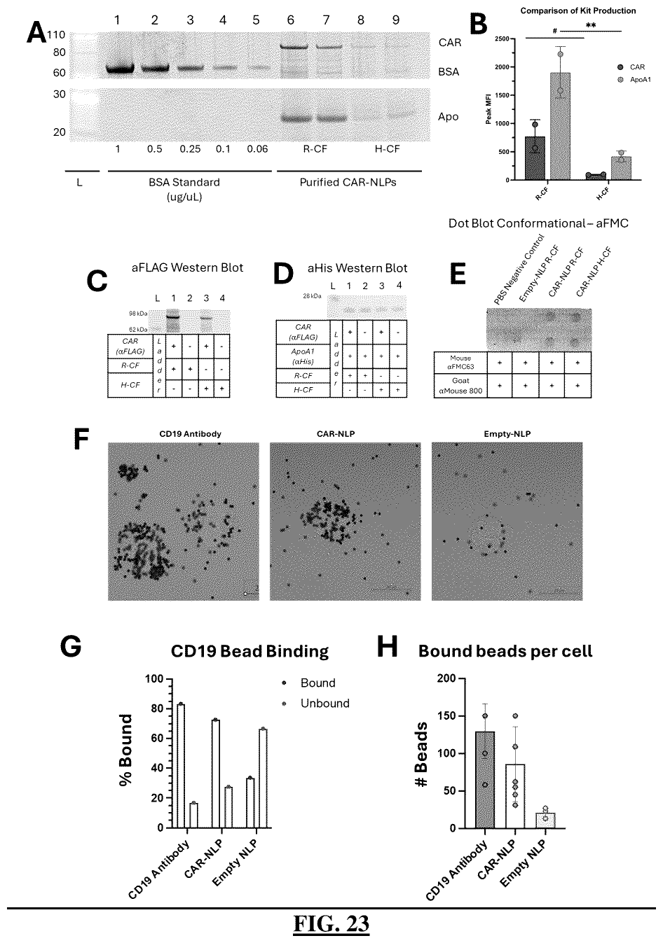

FIG. 23 Panels A-D show results of quantitative SDS PAGE and Western Blot for the purification, quantification, and functional characterization of CAR-containing nanolipoprotein particles (CAR-NLPs) synthesized under optimized reaction conditions. c

In particular, the result of the SDS Page experiments reported in FIG. 23 Panels A-B indicated successful co-incorporation of CAR and Apo, with R-CF reactions producing higher yields overall. CAR: Apo molar ratios in final NLP products were approximately 0.4:1 (H-CF) and 0.75:1 (R-CF), suggesting efficient NLP loading of membrane proteins under these conditions.

The results of the Western blot analysis confirmed reported in FIG. 23 Panels C-D the presence of FLAG-tagged CAR and His-tagged Apo components in the NLP fraction.

Overall, R-CF lysates produced higher yields than the H-CF-lysates (FIG. 23 Panel A, B). While CAR-NLP produced in R-CF lysates appeared to have more CAR protein than H-CF lysates (FIG. 23 Panel B, blue bars), samples were not significantly different through statistical testing (P=0.15). Furthermore, R-CF lysates produced significantly more Apo protein than the H-CF lysates (p=0.005). In short, the production ratios of CAR protein to Apo protein within the CAR-NLP was roughly 0.4:1 for R-CF lysates and 0.2:1 for H-CF lysates, indicating higher CAR embedment within the NLP using R-CF lysates for production (FIG. 23 Panel C).

These findings demonstrate that immuno-nanoparticles can be generated with multiple lysates, including both commercial lysate kits and homemade low endotoxin lysates. Commercial products typically offer greater yield, but CAR: Apo ratios of >0.2 and soluble protein fraction of >0.2 can be obtained with multiple lysates.

### Example 27 Validation of CAR-NLP Functionality

Following production and screening of CAR-NLP, validation of the CAR protein was conducted to verify correct CAR folding within the NLP.

In particular, to verify that CAR proteins embedded in the NLP maintained correct extracellular folding and target binding ability, dot blot assays were performed using a conformation-specific anti-FMC63 antibody. to detect folding of the extracellular region of the CAR construct (FIG. 23 Panel E).

The CAR-NLP reacted positively in this assay, while empty NLPs did not. A large anti-FLAG signal is detected in the CAR-NLP R-CF reactions when compared to the H-CF reactions (FIG. 23 Panel E, lanes 2, 4) while no band is present in the Empty NLP reactions (FIG. 23 Panel E, lanes 3, 5). Overall, CAR-NLP conditions appear to have a higher background signal than the Empty-NLP conditions, with the H-CF reactions (lanes 4) showing less background compared to the R-CF reactions (lane 2). Additionally, NLP formation was assayed using an anti-his antibody for detection of the NLP via the his-tag on the ApoA1 protein. Both R-CF and H-CF reactions gave rise to similar bands of ApoA1 via the anti-his antibody in the CAR-NLP(and Empty-NLP(FIG. 8)

Following dot blot analysis, the CAR-NLP, Empty NLP and a CD19 protein were used to demonstrate binding of the FMC63 region of the CAR to its target, CD19, through a bead binding co-culture assay (FIG. 23 Panel F). Both the CD19 antibody and CAR-NLP showed positive binding visually through microscopy of the CD19+Raji cells they were cocultured with.

Additionally, the empty NLP showed very little binding to the cells (FIG. 23 Panel F). analysis of bound versus unbound beads in the images showed that roughly 80% of CD19 beads and 75% of CAR-NLP beads were bound, while less than 30% of beads were bound to the CD19+ cells in the empty NLP condition (FIG. 23 Panel G). further analysis of the number of beads bound per cell imaged gave a rather large range for the antibody and the CAR-NLP, between 50 and 150 beads per cell, while the empty NLP had less than 25 bound beads in all samples.

Accordingly, the results of FIG. 23, Panels E to G confirm that incorporation of CAR and apolipoprotein components, dot blot data demonstrating conformation-dependent recognition by anti-CAR antibodies, and bead-based binding assays showing selective engagement of CD19-positive target cells by the CAR-NLPs.

These findings demonstrate that the engineered NLPs, synthesized and assembled under defined lipid, temperature, and time conditions, enable presentation of functional CAR constructs capable of target recognition. This supports the utility of the NLP-based system for generation of synthetic immune interfaces, as described herein.

In summary, described herein is a method for generating soluble Immuno-NLPs and in particular CAR-NLPs, a method for delivering an immunoreceptor protein such as a CAR protein to an immune cell using a cargo protein-NLP, a method for delivering an immunoreceptor and in particular a CAR protein to an immune cell using an Immuno-NLP and in particular a CAR-NLP of the disclosure, and related compositions methods and systems.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified NLPs and related uses to additional NLPs formed by other cationic lipids, membrane forming lipids, scaffold proteins, additives, and possibly functionalized amphipathic compounds and membrane proteins according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

1. David, R. M. and A. T. Doherty, Viral Vectors: The Road to Reducing Genotoxicity. Toxicological Sciences, 2017. 155 (2): p. 315-325.
2. Moiani, A., et al., Lentiviral vector integration in the human genome induces alternative splicing and generates aberrant transcripts. The Journal of Clinical Investigation, 2012. 122 (5): p. 1653-1666.
3. Yamagata, Y., et al., Lentiviral Transduction of CD34+ Cells Induces Genome-Wide Epigenetic Modifications. PLOS ONE, 2012. 7 (11): p. c48943.
4. Aranyi, T., et al., Systemic epigenetic response to recombinant lentiviral vectors independent of proviral integration. Epigenetics & Chromatin, 2016. 9 (1): p. 29.
5. Aiuti, A., et al., The committee for advanced therapies' of the European Medicines Agency reflection paper on management of clinical risks deriving from insertional mutagenesis. Human gene therapy Clinical development, 2013. 24 (2): p. 47-54.
6. Baldo, A., et al., General considerations on the biosafety of virus-derived vectors used in gene therapy and vaccination. Current gene therapy, 2013. 13 (6): p. 385-394.
7. FDA Investigating Serious Risk of T-cell Malignancy Following BCMA-Directed or CD19-Directed Autologous Chimeric Antigen Receptor (CAR) T cell Immunotherapies, FDA, Editor. 2023.
8. Subklewe, M., M. von Bergwelt-Baildon, and A. Humpe, Chimeric antigen receptor T cells: a race to revolutionize cancer therapy. Transfusion Medicine and Hemotherapy, 2019. 46 (1): p. 15-24.
9. Patriarchi, T., et al., Nanodelivery of a functional membrane receptor to manipulate cellular phenotype. Scientific Reports, 2018. 8 (1): p. 3556.
10. Alberts, B., et al., Membrane proteins, in Molecular Biology of the Cell. 4^{th} edition. 2002, Garland Science.
11. Zhu, J. and P. Lu, Computational design of transmembrane proteins. Current Opinion in Structural Biology, 2022. 74: p. 102381.
12. Zhang, T., W. Hu, and W. Chen, Plasma membrane integrates biophysical and biochemical regulation to trigger immune receptor functions. Frontiers in Immunology, 2021. 12: p. 613185.
13. Definition, W., Wikipedia definition for transmembrane protein. 2024.
14. Zhou, Y., B. Wang, and F. Yuan, The role of transmembrane proteins in plant growth, development, and stress responses. International Journal of Molecular Sciences, 2022. 23 (21): p. 13627.
15. Qin, V. M., et al., Chimeric antigen receptor beyond CAR-T cells. Cancers, 2021. 13 (3): p. 404.
16. cell, W. C.-T. 2024; Available from: https://en.wikipedia.org/wiki/CAR_T_cell at the filing date of the disclosure.
17. Maldini, C. R., G. I. Ellis, and J. L. Riley, CAR T cells for infection, autoimmunity and allotransplantation. Nature Reviews Immunology, 2018. 18 (10): p. 605-616.
18. Aghajanian, H., et al., Targeting cardiac fibrosis with engineered T cells. Nature, 2019. 573 (7774): p. 430-433.
19. Amor, C., et al., Senolytic CAR T cells reverse senescence-associated pathologies. Nature, 2020. 583 (7814): p. 127-132.
20. Library, S. D., 2024.
21. Mazinani, M. and F. Rahbarizadeh, CAR-T cell potency: from structural elements to vector backbone components. Biomarker research, 2022. 10 (1): p. 70.
22. Nix, M. A. and A. P. Wiita, Alternative target recognition elements for chimeric antigen receptor (CAR) T cells: beyond standard antibody fragments. Cytotherapy, 2024.
23. Ramilowski, J. A., et al., A draft network of ligand-receptor-mediated multicellular signalling in human. Nature communications, 2015. 6 (1): p. 7866.
24. Jin, S., et al., Inference and analysis of cell-cell communication using CellChat. Nature Communications, 2021. 12 (1): p. 1088.
25. Zoine, J. T., et al., Peptide-scFv antigen recognition domains effectively confer CAR T cell multiantigen specificity. Cell Reports Medicine, 2024.
26. Hughes-Parry, H. E., R. S. Cross, and M. R. Jenkins, The evolving protein engineering in the design of chimeric antigen receptor T cells. International Journal of Molecular Sciences, 2019. 21 (1): p. 204.
27. Zhang, C., et al., Engineering car-t cells. Biomarker research, 2017. 5: p. 1-6.
28. Sterner, R. C. and R. M. Sterner, CAR-T cell therapy: current limitations and potential strategies. Blood cancer journal, 2021. 11 (4): p. 69.
29. Ahmad, U., et al., Chimeric antigen receptor T cell structure, its manufacturing, and related toxicities; A comprehensive review. Advances in Cancer Biology-Metastasis, 2022. 4: p. 100035.
30. Fujiwara, K., et al., Hinge and transmembrane domains of chimeric antigen receptor regulate receptor expression and signaling threshold. Cells, 2020. 9 (5): p. 1182.
31. Elazar, A., et al., De novo-designed transmembrane domains tune engineered receptor functions. Elife, 2022. 11: p. e75660.
32. Rafiq, S., C. S. Hackett, and R. J. Brentjens, Engineering strategies to overcome the current roadblocks in CAR T cell therapy. Nature reviews Clinical oncology, 2020. 17 (3): p. 147-167.
33. Sievers, N. M., J. Dorrie, and N. Schaft, CARs: beyond T cells and T cell-derived signaling domains. International Journal of Molecular Sciences, 2020. 21 (10): p. 3525.
34. Chang, Z. L. and Y. Y. Chen, CARs: synthetic immunoreceptors for cancer therapy and beyond. Trends in molecular medicine, 2017. 23 (5): p. 430-450.
35. Polavarapu, K. and T. Shroka, Combining CAR T-cell and immune checkpoint inhibitor therapy, a promising, yet unproven immuno-oncology approach. Journal of Student Research, 2023. 12 (1).
36. Weinkove, R., et al., Selecting costimulatory domains for chimeric antigen receptors: functional and clinical considerations. Clinical & translational immunology, 2019. 8 (5): p. e1049.
37. Boucher, J. C., et al., CD28 costimulatory domain-targeted mutations enhance chimeric antigen receptor T-cell function. Cancer immunology research, 2021. 9 (1): p. 62-74.
38. Chu, G. J., et al., The 4-1BBζ costimulatory domain in chimeric antigen receptors enhances CD8+ T-cell functionality following T-cell receptor stimulation. Cancer Cell International, 2023. 23 (1): p. 327.
39. Selli, M. E., et al., Costimulatory domains direct distinct fates of CAR-driven T-cell dysfunction. Blood, The Journal of the American Society of Hematology, 2023. 141 (26): p. 3153-3165.
40. He, W., et al., Cell-free expression of functional receptor tyrosine kinases. Sci Rep, 2015. 5: p. 12896.
41. Umbach, S., et al., Transfer mechanism of cell-free synthesized membrane proteins into mammalian cells. Frontiers in bioengineering and biotechnology, 2022. 10.
42. Seo, M. J., et al., Engineering antibody fragments to fold in the absence of disulfide bonds. Protein Sci, 2009. 18 (2): p. 259-67.
43. Köck, Z., et al., Cryo-EM structure of cell-free synthesized human histamine 2 receptor/Gs complex in nanodisc environment. Nature Communications, 2024. 15 (1): p. 1831.
44. Cappuccio, J. A., et al., Cell-free Co-expression of Functional Membrane Proteins and Apolipoprotein, Forming Soluble Nanolipoprotein Particles. Molecular & Cellular Proteomics, 2008. 7 (11): p. 2246-2253.
45. Gao, T. J., et al., Characterizing diffusion dynamics of a membrane protein associated with nanolipoproteins using fluorescence correlation spectroscopy. Protein Science, 2011. 20 (2): p. 437-447.
46. Katzen, F., et al., Insertion of membrane proteins into discoidal membranes using a cell-free protein expression approach. Journal of Proteome Research, 2008. 7 (8): p. 3535-3542.
47. FRECHET, J. M. and D. A. TOMALIA, Dendrimers and Other Dendritic Polymers. 2001.
48. Fischer, N. O., et al., Conjugation to Nickel-Chelating Nanolipoprotein Particles Increases the Potency and Efficacy of Subunit Vaccines to Prevent West Nile Encephalitis. Bioconjugate Chemistry, 2010. 21 (6): p. 1018-1022.
49. Fischer, N. O., et al., Immobilization of His-Tagged Proteins on Nickel-Chelating Nanolipoprotein Particles. Bioconjugate Chemistry, 2009. 20 (3): p. 460-465.
50. Sperling, R. A. and W. J. Parak, Surface modification, functionalization and bioconjugation of colloidal inorganic nanoparticles. Philos Trans A Math Phys Eng Sci, 2010. 368 (1915): p. 1333-83.
51. He, H., et al., Arachidonic Acid-Modified Lovastatin Discoidal Reconstituted High Density Lipoprotein Markedly Decreases the Drug Leakage during the Remodeling Behaviors Induced by Lecithin Cholesterol Acyltransferase. Pharmaceutical Research, 2014. 31 (7): p. 1689-1709.
52. Weilhammer, D., et al., Enhancement of antigen-specific CD4+ and CD8+ T cell responses using a self-assembled biologic nanolipoprotein particle vaccine. Vaccine, 2017. 35 (11): p. 1475-1481.
53. Janeway Jr, C. A., et al., The production of armed effector T cells, in Immunobiology: The Immune System in Health and Disease. 5^{th} edition. 2001, Garland Science.
54. Petrus-Reurer, S., et al., Immunological considerations and challenges for regenerative cellular therapies. Communications biology, 2021. 4 (1): p. 798.
55. Hammel, J. H., et al., Modeling immunity in vitro: slices, chips, and engineered tissues. Annual review of biomedical engineering, 2021. 23 (1): p. 461-491.
56. Harrer, D. C., J. Dörrie, and N. Schaft, CARs and Drugs: Pharmacological Ways of Boosting CAR-T-Cell Therapy. International Journal of Molecular Sciences, 2023. 24 (3): p. 2342.
57. Sadelain, M., R. Brentjens, and I. Rivière, The promise and potential pitfalls of chimeric antigen receptors. Current Opinion in Immunology, 2009. 21 (2): p. 215-223.
58. Tomasik, J., M. Jasiński, and G. W. Basak, Next generations of CAR-T cells-new therapeutic opportunities in hematology? Front Immunol, 2022. 13: p. 1034707.
59. He, W., et al., Nanolipoprotein-Mediated Her2 Protein Transfection Induces Malignant Transformation in Human Breast Acinar Cultures. ACS Omega, 2021. 6 (44): p. 29416-29423.
60. Murphy, K. M., et al., Janeway's Immunobiology. 7^{th} ed. 2022: W. W. Norton.
61. Cappuccio, J. A., et al., Cell-free Co-expression of Functional Membrane Proteins and Apolipoprotein, Forming Soluble Nanolipoprotein Particles. Molecular &amp; Cellular Proteomics, 2008. 7 (11): p. 2246-2253.
62. Shelby, M. L., et al., Cell-Free Co-Translational Approaches for Producing Mammalian Receptors: Expanding the Cell-Free Expression Toolbox Using Nanolipoproteins. Frontiers in pharmacology, 2019. 10: p. 744-744.
63. Lewis, R. N. A. H., Y.-P. Zhang, and R. N. McElhaney, Calorimetric and spectroscopic studies of the phase behavior and organization of lipid bilayer model membranes composed of binary mixtures of dimyristoylphosphatidylcholine and dimyristoylphosphatidylglycerol. Biochimica et Biophysica Acta (BBA)-Biomembranes, 2005. 1668 (2): p. 203-214.
64. Jewett, M. C. and J. R. Swartz, Mimicking the Escherichia coli cytoplasmic environment activates long-lived and efficient cell-free protein synthesis. Biotechnology and Bioengineering, 2004. 86 (1): p. 19-26.

1. An immuno-nanolipoprotein particle (Immuno-NLP) comprising:
one or more membrane forming lipid, a scaffold protein, and an immune cell transmembrane protein, comprising an extracellular domain, a hydrophobic transmembrane domain, and an intracellular domain,
wherein the one or more membrane forming lipids are arranged in a discoidal membrane lipid bilayer stabilized by the scaffold protein, in a configuration in which the transmembrane protein is attached to the membrane lipid bilayer through interactions of the hydrophobic transmembrane domain of the immunoreceptor with the membrane lipid bilayer, and where the immuno-nanolipoprotein particle presents a functional extracellular domain of the immune cell transmembrane protein.

2. The immuno-nanolipoprotein particle of claim 1, wherein the immune cell transmembrane protein comprises one or more immunoreceptors.

3. The immuno-nanolipoprotein particle of claim 1, wherein the immune cell transmembrane protein comprises one or more of CD4, CD8, CD19, CD20, CD45, CD56, CD80, CD86 cytokine receptors such as Interleukin-2 receptor (IL-2R): Interleukin-6 receptor (IL-6R); and Tumor necrosis factor receptor (TNFR):, checkpoint inhibitors, such as PD-1 (Programmed Cell Death Protein 1).

4. The immuno-nanolipoprotein particle of claim 1, wherein the immune cell transmembrane protein comprises a CAR protein having an Antigen Recognition Domain (herein also CAR-ARD), a Transmembrane Domain (herein also CAR-TMD); and optionally, intracellular Signaling Domains (herein also CAR-ISD).

5. The immuno-nanolipoprotein particle of claim 4, further comprising A Hinge Region (herein also CAR-HGR) and a Costimulatory Domains (herein also CAR-CSD).

6. The immuno-nanolipoprotein particle of claim 4, wherein the CAR proteins is a first-generation, second-and third-generation CARs or fourth generation CARs.

7. The immuno-nanolipoprotein particle of claim 6, wherein the CAR protein comprise single chain variable fragments (scFv) derived monoclonal antibodies, fused to CD33 transmembrane and intracellular domain.

8. The immuno-nanolipoprotein particle of claim 6, wherein the CAR protein is configured to bind to CD3, CD19, CD22, CD30, CD123, B cell maturation antigen (BCMA), GD2, mesothelin, EGVRVIII, HER2, e-MET, PD-L1, and/or other tumor associated antigens.

9. The immuno-nanolipoprotein particle of claim 6, wherein the CAR protein is configured to bind to molecules expressed on the surface of tumor cells, including CD20, CD22, CD33, CD2, CD3, CD4, CD5, CD7, CD8, CD45, CD52, CD38, CS-1, TIM3, CD123, mesothelin, folate receptor, HER2-neu, epidermal-growth factor receptor, and epidermal growth factor receptor.

10. The immuno-nanolipoprotein particle of claim 6, wherein the CAR protein is configured to bind to molecules expressed on the surface of B-cells such as CD19, CD20, CD22, CD72, and CD7.

11. The immuno-nanolipoprotein particle of claim 6, wherein the CAR protein is configured to bind to molecules expressed on the surface of T cells such as CD2, CD3, CD4, CD5, CD7, and/or CD8.

12. The immuno-nanolipoprotein particle of claim 6, wherein the CAR protein is configured to bind to molecules expressed on the surface of eosinophils mast-cells and basophils such as CD9, CD63, and CD203.

13. The immuno-nanolipoprotein particle of claim 6, wherein the CAR protein is configured to bind to molecules expressed on fibroblast or activated myofibroblasts such as fibroblast associated protein.

14. The immuno-nanolipoprotein particle of claim 6, wherein the CAR protein is configured to bind to molecules expressed on senescent cells including urokinase plasminogen activator receptor.

15. The immuno-nanolipoprotein particle of claim 6, wherein the CAR protein is configured to bind molecules expressed on the coat of pathogens that can be recognized by the immune system.

16. The immuno-nanolipoprotein particle of claim 6, wherein the antigen recognition domain of the CAR comprises an scFv, and the transmembrane domain comprises the CD28 transmembrane domains, and the intracellular region comprises CD3ζ stimulatory domain and CD28 costimulatory domain.

17. The immuno-nanolipoprotein particle of claim 1, wherein the membrane forming lipid comprises at least one phospholipid, selected from soy phosphatidylcholine, egg phosphatidylcholine, soy phosphatidylglycerol, egg phosphatidylglycerol, palmitoyl-oleoyl-phosphatidylcholine distearoylphosphatidylcholine, distearoylphosphatidylglycerol phosphatidylcholine, phosphatidylglycerol, sphingomyelin, phosphatidylserine, phosphatidic acid, phosphatidylethanolamine, lysolecithin, lysophosphatidylethanolamine,
phosphatidylinositol, cephalin, cardiolipin, cerebrosides, dicetylphosphate,
dioleoylphosphatidylcholine, dipalmitoylphosphatidylcholine, dipalmitoylphosphatidylglycerol, dioleoylphosphatidylglycerol, stearoyl-palmitoyl-phosphatidylcholine, di-palmitoyl-phosphatidylethanolamine, distearoyl-phosphatidylethanolamine, di-myrstoyl-phosphatidylserine, di-myrstoyl-phosphatidylcholine and dioleyl-phosphatidylcholine.

18. The immuno-nanolipoprotein particle of claim 1, wherein the scaffold protein is one or more of a human derived apoE4, a truncated version of human derived apoE4, a human derived apoE3, a truncated versions of human derived apoE3, a human derived apoE2, a truncated version of human derived apoE2, a human derived apoA1, a truncated version of human derived apoAl, a mouse derived apoE4, a truncated version of mouse derived apoE4, mouse derived apoE3, a truncated version of mouse derived apoE3, a mouse derived apoE2, a truncated version of mouse derived apoE2, a mouse derived apoA1, a truncated version of mouse derived apoA1, a rat derived apoE4, a truncated version of rat derived apoE4, a rat derived apoE3, a truncated version of rat derived apoE3, a rat derived apoE2, a truncated version of rat derived apoE2, a rat derived apoAl, a truncated version of rat derived apoA1, a lipophorins, a synthetic cyclic peptide mimicking an apolipoprotein function.

19. The immuno-nanolipoprotein particle of claim 1, further comprising functionalized amphipathic compounds.

20. The immuno-nanolipoprotein particle of claim 1, further comprising a telodendrimer.

21. An immuno-nanolipoprotein particle composition comprising one or more immune cell transmembrane protein, and one or more NLPs in which one or more membrane forming lipids are arranged in a discoidal membrane lipid bilayer stabilized by the scaffold protein, wherein an immune cell transmembrane protein of one or more immune cell transmembrane proteins is attached to the membrane lipid bilayer of an NLP of the one or more NLPs through interactions of the hydrophobic transmembrane domain of the transmembrane protein with the membrane lipid bilayer to form an immuno-nanolipoprotein particle according to claim 1,
wherein the immune cell transmembrane protein and the NLP are in a suspension that is >50% and possibly >90% soluble in a buffered aqueous solution, and
wherein in the immune cell transmembrane protein and the NLP are in molar ratio of immunoreceptor to NLP of at least 1:10.

22. The immuno-nanolipoprotein particle composition of claim 21, comprising a ratio of immune cell transmembrane protein to NLPs of 0.2:1.

23. The immuno-nanolipoprotein particle composition of claim 21, comprising a scaffold protein to NLP ratio of about 2 and an immune transmembrane protein to: scaffold protein ratio of about 0.1 to 2.5.

24. An immuno-nanolipoprotein particle treatment formulation comprising the immuno-nanolipoprotein particle composition of claim 21, comprising no detectable amount of an insoluble immune cell transmembrane protein, and an imidazole buffer.


SEQUENCE LISTING (7 sequences)

SEQ ID NO: 1 - DNA, 606 bp, synthetic construct
  source 1..606: mol_type = other DNA
catatgctgaatctgctggaaaactgggacaccctgggctccacggtgtcacagctgcaagaacgcctgggtccgctgacgcgtgatttttgggacaacctggaaaaagaaaccgattgggttcgccaggaaatgaataaggacctggaagaagtgaaacagaaggttcaaccgtatctggatgaatttcagaaaaagtggaaagaagacgtcgaactgtaccgtcagaaggtggcaccgctgggcgctgaactgcaagaatccgcacgccagaaactgcaagaactgcaaggtcgtctgtcaccggttgctgaagaatttcgtgatcgcatgcgtacgcatgtggattcgctgcgcacccaactggcaccgcactctgaacagatgcgcgaaagtctggcgcaacgtctggccgaactgaaaagtaacccgaccctgaatgaataccatacccgtgccaaaacgcacctgaagaccctgggtgaaaaagcacgtccggcgctggaagacctgcgtcattctctgatgccgatgctggaaaccctgaaaacccaagtccagtcggtgattgacaaagcaagcgaaaccctgacggcacagggatcc

SEQ ID NO: 2 - protein, 198 aa, synthetic construct
LNLLENWDTLGSTVSQLQERLGPLTRDFWDNLEKETDWVRQEMNKDLEEVKQKVQPYLDEFQKKWKEDVELYRQKVAPLGAELQESARQKLQELQGRLSPVAEEFRDRMRTHVDSLRTQLAPHSEQMRESLAQRLAELKSNPTLNEYHTRAKTHLKTLGEKARPALEDLRHSLMPMLETLKTQVQSVIDKASETLTAQ

SEQ ID NO: 3 - DNA, 555 bp, synthetic construct
  source 1..555: mol_type = other DNA
catatgggtgaaccggaagtgaccgatcaactggaatggcaatctaatcaaccgtgggaacaagccctgaaccgtttttgggactatctgcgctgggtgcaaaccctgagcgatcaggttcaagaagaactgcagagctctcaagttacccaggaactgacggcactgatggaagacaccatgacggaagtcaaagcttataaaaaggaactggaagaacagctgggcccggtcgcagaagaaacgcgtgctcgcctgggtaaagaagtgcaagcagcacaggcacgtctgggtgcagatatggaagacctgcgtaaccgcctgggtcaataccgtaatgaagtgcataccatgctgggccagagtacggaagaaattcgtgcgcgcctgtccacccacctgcgtaaaatgcgtaagcgcctgatgcgcgatgcggaagacctgcagaaacgtctggccgtttataaggcaggcgctcgcgaaggtgccgaacgtggtgtgtcggcaatccgtgaacgcctgggtccgctggttgaacaaggtcgtcagggatcc

SEQ ID NO: 4 - protein, 185 aa, synthetic construct
HMGEPEVTDQLEWQSNQPWEQALNRFWDYLRWVQTLSDQVQEELQSSQVTQELTALMEDTMTEVKAYKKELEEQLGPVAEETRARLGKEVQAAQARLGADMEDLRNRLGQYRNEVHTMLGQSTEEIRARLSTHLRKMRKRLMRDAEDLQKRLAVYKAGAREGAERGVSAIRERLGPLVEQGRQGS

SEQ ID NO: 5 - DNA, 594 bp, synthetic construct
  source 1..594: mol_type = other DNA
ctgaatctcctggaaaactgggacactctgggttcaaccgttagtcagctgcaggaacggctgggcccattgactcgggacttctgggataacctggagaaagaaacagattgggtgagacaggagatgaacaaggacctagaggaagtgaaacagaaggtgcagccctacctggacgaattccagaagaaatggaaagaggatgtggagctctaccgccagaaggtggcgcctctgggcgccgagctgcaggagagcgcgcgccagaagctgcaggagctgcaagggagactgtcccctgtggctgaggaatttcgcgaccgcatgcgcacacacgtagactctctgcgcacacagctagcgccccacagcgaacagatgcgcgagagcctggcccagcgcctggctgagctcaagagcaaccctaccttgaacgagtaccacaccagggccaaaacccacctgaagacacttggcgagaaagccagacctgcgctggaggacctgcgccatagtctgatgcccatgctggagacgcttaagacccaagtccagagtgtgatcgacaaggccagcgagactctgactgcccag

SEQ ID NO: 6 - DNA, 594 bp, synthetic construct
  source 1..594: mol_type = other DNA
ctgaatctgctggaaaactgggacaccctgggctccacggtgtcacagctgcaagaacgcctgggtccgctgacgcgtgatttttgggacaacctggaaaaagaaaccgattgggttcgccaggaaatgaataaggacctggaagaagtgaaacagaaggttcaaccgtatctggatgaatttcagaaaaagtggaaagaagacgtcgaactgtaccgtcagaaggtggcaccgctgggcgctgaactgcaagaatccgcacgccagaaactgcaagaactgcaaggtcgtctgtcaccggttgctgaagaatttcgtgatcgcatgcgtacgcatgtggattcgctgcgcacccaactggcaccgcactctgaacagatgcgcgaaagtctggcgcaacgtctggccgaactgaaaagtaacccgaccctgaatgaataccatacccgtgccaaaacgcacctgaagaccctgggtgaaaaagcacgtccggcgctggaagacctgcgtcattctctgatgccgatgctggaaaccctgaaaacccaagtccagtcggtgattgacaaagcaagcgaaaccctgacggcacag

SEQ ID NO: 7 - DNA, 594 bp, synthetic construct
  source 1..594: mol_type = other DNA
ctgaacctgctggaaaactgggacaccctgggttctaccgtttctcagctgcaggaacgtctgggtccgctgacccgtgacttctgggacaacctggaaaaagaaaccgactgggttcgtcaggaaatgaacaaagacctggaagaagttaaacagaaagttcagccgtacctggacgaattccagaaaaaatggaaagaagacgttgaactgtaccgtcagaaagttgcgccgctgggtgcggaactgcaggaatctgcgcgtcagaaactgcaggaactgcagggtcgtctgtctccggttgcggaagaattccgtgaccgtatgcgtacccacgttgactctctgcgtacccagctggcgccgcactctgaacagatgcgtgaatctctggcgcagcgtctggcggaactgaaatctaacccgaccctgaacgaataccacacccgtgcgaaaacccacctgaaaaccctgggtgaaaaagcgcgtccggcgctggaagacctgcgtcactctctgatgccgatgctggaaaccctgaaaaccaaagcgcagtctgttatcgacaaagcgtctgaaaccctgaccgcgcag